(19) United States
(12) Patent Application Publication
(10) Pub. No.: US 2023/0321279 A1
(43) Pub. Date: Oct. 12, 2023

(54) REGULATORY NUCLEIC ACID SEQUENCES

(71) Applicant: ASKLEPIOS BIOPHARMACEUTICAL, INC., Research Triangle Park, NC (US)

(72) Inventors: Anne BRAAE, Midlothian (GB); Juan Manuel Iglesias, Midlothian (GB); Michael L. Roberts, Midlothian (GB); Sinclair Cooper, Midlothian (GB)

(73) Assignee: ASKLEPIOS BIOPHARMACEUTICAL, INC., Research Triangle Park, NC (US)

(21) Appl. No.: 18/024,211

(22) PCT Filed: Sep. 3, 2021
(86) PCT No.: PCT/GB2021/052273

§ 371 Date: Mar. 1, 2023

(30) Foreign Application Priority Data
Sep. 4, 2020 (GB) 2013940.8

(51) Int. Cl. A61K 48/00; C12N 15/86; C12N 9/02

(52) U.S. Cl. CPC A61K 48/0058; C12N 15/86; C12N 2830/008; C12N 2750/14143; C12N 9/0071

(57) ABSTRACT

The present invention relates to regulatory nucleic acid sequences, in particular NS-specific promoters, cell specific promoters, multi-cell specific promoters, and elements thereof. The invention also relates to expression constructs, vectors, virions, pharmaceutical compositions and cells comprising such promoters and to methods of their use. The regulatory nucleic acid sequences are of particular utility for gene therapy applications.

### FIELD OF THE INVENTION

The present invention relates to regulatory nucleic acid sequences, in particular nervous system-specific promoters, cell specific promoters, multi-cell specific promoters, and elements thereof. The invention also relates to expression constructs, vectors, virions, pharmaceutical compositions and cells comprising such promoters and to methods of their use. The regulatory nucleic acid sequences are of particular utility for gene therapy applications.

### BACKGROUND OF THE INVENTION

The following discussion is provided to aid the reader in understanding the disclosure and does not constitute any admission as to the contents or relevance of the prior art.

Following extensive study of the internal mechanisms of gene regulation within the body, research focus has recently shifted to regulation of gene expression by introducing exogenous nucleic acid sequences into cells.

This is done conventionally in research and bioprocessing, wherein the nucleic acid sequence of a desired expression product operably liked to a promoter is introduced into a production cell line, often in the form of a vector.

In the field of gene therapy, this has been of particular interest for genetic disorders such as single gene disorders (or Mendelian disorders) which are caused by the presence of a faulty gene into the cells of a patient. Introduction of the nucleic acid sequence of a wild type allele of the faulty gene operably linked to a promoter into the cells of a patient is a favourable treatment option as it can cure the condition while conventional medicines can only address the symptoms.

Additionally, or alternatively, introduction of a nucleic acid which can suppress the faulty gene into the cells of a patient may be beneficial when the faulty gene has a dominant negative mutation which acts antagonistically on a wild type allele. Finally, introducing a nucleic acid sequence which can counteract or alleviate the detrimental effects of the faulty gene into the cells of a patient is also alternative or adjuvant therapeutic option.

In gene therapy, controlling the expression of the exogenous nucleic acid which has been introduced into the cells is of paramount importance for the health and safety of the patients. The level of an expression product not only needs to be within a therapeutic window but also the expression needs to be within a required tissue or even a specific region within the required tissue for the treatment to be effective. Additionally, expression may need to be restricted to a specific cell type or a multiple cell types in order to avoid side effects. Expression outside the therapeutic window (i.e. lower or higher) or expression outside the therapeutic region, or even outside the specific cell or combination of cells, may not be useful therapeutically or even be deleterious.

Huntington's disease is caused by an autosomal dominant mutation on one of the two inherited copies of the huntingtin gene. This neurodegenerative disease is associated with progressively worsening motor dysfunction, impairment of cognitive abilities caused by the death of neurones. There is no cure for Huntington's disease and treatments are directed at alleviating the symptoms. Since the disease is caused by an autosomal dominant mutation which acts antagonistically on the wild type huntingtin allele present in the body, replacement therapy by introduction of a wild type allele is unlikely to be particularly beneficial.

One therapeutic option for Huntington's disease is to introduce a nucleic acid which can suppress the faulty huntingtin gene into the cells of a patient. In non-disease condition, huntingtin is expressed throughout the body and is very highly expressed in the CNS where it is found in all neurones and also glial cells (Schulte and Littleton, 2011). Loss of function due to the autosomal dominant mutation in the faulty huntingtin gene and/or gain of function of the faulty huntingtin gene contribute not only to neuronal death but also to glia disfunction. To support that, in addition to neuronal death, reactive microglia and activated astrocytes and oligodendroglia have been observed in the brains of Huntington's patients (Schulte and Littleton, 2011). Therefore, it is desirable to express nucleic acids which can suppress the faulty huntingtin gene not only in all neurones, but also in some or all glial cells.

Therefore, there is a need for promoters driving expression in the nervous system (NS), specifically the CNS, particularly in specific cell types such as neurones as well as neuronal supporting cells, including some or all glial cells, oligodendrocytes, interneurons, and the like. Expression in the nervous system (NS) includes expression in the cells of the central nervous system (CNS) and/or peripheral nervous system (PNS). Expression in the CNS includes expression in cells in the brain and spinal cord, and includes neuronal cells as well as non-neuronal cells, interneurons, or neuronal supporting cells. Expression in the PNS includes expression in cells in the peripheral nervous system, and includes neuronal cells as well as non-neuronal cells, interneurons, or neuronal supporting cells.

Another therapeutic option is introducing a nucleic acid sequence which can counteract or alleviate the detrimental effects of the faulty huntingtin gene into the cells of a patient. Huntington's disease is associated with abnormal cholesterol metabolism with persistent cholesterol accumulation in the brain despite reduced synthesis (Boussicault et al., 2016). Since the blood brain barrier is not permeable to cholesterol, the cholesterol homeostasis of the CNS is separate to the rest of the body, and can therefore be targeted separately. In the CNS, astrocytes synthesise cholesterol and neurones catabolise it via the neurone-specific enzyme CYP46A1 (Boussicault et al., 2016). Additionally, cholesterol is metabolised in oligodendrocytes to produce myelin basic protein (MBP). The CYP46A1 expression level is reduced in Huntington's disease and the CYP46A1 enzyme level is apparently insufficient to overcome the cholesterol accumulation in the brain (Boussicault et al., 2016). To support this, CYP46A1 expression by the ubiquitous promoters CMV or CAG was found to regulate the cholesterol metabolism in the brain in a mouse Huntington's disease model (Kacher et al., 2019). Therefore, expressing additional CYP46A1 in neuronal cells and/or introducing CYP46A1 (and therefore the ability to catabolise cholesterol) into glial cells is able to regulate the cholesterol metabolism in the brain.

While expression of CYP46A1 in the CNS appears to be an attractive therapeutic option for Huntington's disease, in a gene therapy setting in humans it is preferable to introduce the nucleic acid encoding the CYP46A1 in the NS via minimally invasive systemic administration such as intravenous or intraarterial (e.g. intra-carotid) administration. However, a systemic administration of a nucleic acid encoding the CYP46A1 enzyme operably linked to a ubiquitous promoter may have undesirable side effects on the cholesterol metabolism outside of the NS due to expression of CYP46A1 in non-NS cells.

Therefore, there is a need for NS-specific promoters driving expression in neurones and some or all glial cells.

Other diseases of the NS are also suitable targets for gene therapy. In some such diseases, targeted expression of a therapeutic gene in specific NS cells may be desired, and in others, a more generalised, non-specific expression in the NS, preferably the CNS, may be suitable.

One or more aspects of the present invention are intended to address one or more of the above-mentioned problems.

### SUMMARY OF THE INVENTION

In a first aspect of the present invention, there is provided a synthetic nervous system (NS)-specific promoter comprising or consisting of at least one of the following cis-regulatory elements (CREs) or functional variants thereof:
CRE0001_S100B (SEQ ID NO: 22) or a functional variant thereof;
CRE0002_S100B (SEQ ID NO: 24) or a functional variant thereof;
CRE0005_GFAP (SEQ ID NO: 19) or a functional variant thereof;
CRE0007_GFAP (SEQ ID NO: 20) or a functional variant thereof;
CRE0009_S100B (SEQ ID NO: 23) or a functional variant thereof;
CRE0012_Arc (SEQ ID NO: 21) or a functional variant thereof;
CRE0006_GFAP (SEQ ID NO: 27) or functional variant thereof;
CRE0008_GFAP (SEQ ID NO: 28) or functional variant thereof;
CRE0006_AQP4 (SEQ ID NO: 37) or functional variant thereof; and
CRE0008_AQP4 (SEQ ID NO: 38) or functional variant thereof
operably linked to a promoter element, optionally wherein the promoter element is selected from:
CRE0003_CEND1_mp (SEQ ID NO: 25) or a functional variant thereof;
CRE0009_SYN1_mp (SEQ ID NO: 26) or a functional variant thereof,
CRE0001_CEND1_mp (SEQ ID NO: 29) or a functional variant thereof;
CRE0002_CEND1_mp (SEQ ID NO: 30) or a functional variant thereof;
CRE0008_SYN1_mp (SEQ ID NO: 31) or a functional variant thereof;
CRE0013_SYN1_mp (SEQ ID NO: 32) or a functional variant thereof;
CRE0015_GAP43_mp (SEQ ID NO: 39) or a functional variant thereof;
CRE0016_Eno2_mp (SEQ ID NO: 40) or a functional variant thereof; and/or
CRE0004_GFAP_mp (SEQ ID NO: 41) or a functional variant thereof.

In some embodiments, the nervous system (NS)-specific promoter is active in both the central nervous system (CNS) and peripheral nervous system (PNS). In some embodiments, the NS-specific promoter is active in the CNS, i.e. a CNS-specific promoter. In some embodiments, the NS-specific promoter is active in the CNS but not the PNS. In some embodiments, the NS-specific promoter is active in the PNS, i.e. a PNS-specific promoter. In some embodiments, the NS-specific promoter is active in the PNS but not the CNS.

Suitably, there is provided a synthetic NS-specific promoter comprising or consisting of at least one of the following cis-regulatory elements (CREs) or functional variants thereof:
CRE0001_S100B (SEQ ID NO: 22) or a functional variant thereof;
CRE0002_S100B (SEQ ID NO: 24) or a functional variant thereof;
CRE0005_GFAP (SEQ ID NO: 19) or a functional variant thereof;
CRE0007_GFAP (SEQ ID NO: 20) or a functional variant thereof;
CRE0009_S100B (SEQ ID NO: 23) or a functional variant thereof;
CRE0012_Arc (SEQ ID NO: 21) or a functional variant thereof;
CRE0006_GFAP (SEQ ID NO: 27) or functional variant thereof;
CRE0008_GFAP (SEQ ID NO: 28) or functional variant thereof;
CRE0006_AQP4 (SEQ ID NO: 37) or functional variant thereof; and
CRE0008_AQP4 (SEQ ID NO: 38) or functional variant thereof
operably linked to a promoter element selected from:
CRE0003_CEND1_mp (SEQ ID NO: 25) or a functional variant thereof;
CRE0009_SYN1_mp (SEQ ID NO: 26) or a functional variant thereof,
CRE0001_CEND1_mp (SEQ ID NO: 29) or a functional variant thereof;
CRE0002_CEND1_mp (SEQ ID NO: 30) or a functional variant thereof;
CRE0008_SYN1_mp (SEQ ID NO: 31) or a functional variant thereof;
CRE0013_SYN1_mp (SEQ ID NO: 32) or a functional variant thereof;
CRE0015_GAP43_mp (SEQ ID NO: 39) or a functional variant thereof;
CRE0016_Eno2_mp (SEQ ID NO: 40) or a functional variant thereof; and/or
CRE0004_GFAP_mp (SEQ ID NO: 41) or a functional variant thereof.

Functional variants are defined hereinbelow. Suitably, the synthetic NS-specific promoter may comprise a sequence which is at least 60%, 65%, 70%, 75%, 80%, 90%, 95%, 96%, 97%, 98% or 99% identical to any one of SEQ ID NOs: 19-32, 37-41. Suitably the synthetic NS-specific promoter may comprise a sequence which is at least 60%, 65%, 70%, 75%, 80%, 90%, 95%, 96%, 97%, 98% or 99% identical to any one of SEQ ID NOs: 19, 20, 21, 22, 23, 24, 27, 28, 37, 38 operably linked to a sequence which is at least 60%, 65%, 70%, 75%, 80%, 90%, 95%, 96%, 97%, 98% or 99% identical to any one of SEQ ID NOs: 25, 26, 29-32, 39-41.

In one embodiment, there is provided a NS-specific promoter comprising or consisting of at least one of the following cis-regulatory elements (CREs):
CRE0001_S100B (SEQ ID NO: 22) or a sequence which is at least 60%, 65%, 70%, 75%, 80%, 90%, 95%, 96%, 97%, 98% or 99% identical thereto;
CRE0007_GFAP (SEQ ID NO: 20) or a sequence which is at least 60%, 65%, 70%, 75%, 80%, 90%, 95%, 96%, 97%, 98% or 99% identical thereto;
CRE0009_S100B (SEQ ID NO: 23) or a sequence which is at least 60%, 65%, 70%, 75%, 80%, 90%, 95%, 96%, 97%, 98% or 99% identical thereto;
CRE0002_S100B (SEQ ID NO: 24) or a sequence which is at least 60%, 65%, 70%, 75%, 80%, 90%, 95%, 96%, 97%, 98% or 99% identical thereto;
CRE0005_GFAP (SEQ ID NO: 19) or a sequence which is at least 60%, 65%, 70%, 75%, 80%, 90%, 95%, 96%, 97%, 98% or 99% identical thereto;
CRE0012_Arc (SEQ ID NO: 21) or a sequence which is at least 60%, 65%, 70%, 75%, 80%, 90%, 95%, 96%, 97%, 98% or 99% identical thereto;
CRE0006_GFAP (SEQ ID NO: 27) or a sequence which is at least 60%, 65%, 70%, 75%, 80%, 90%, 95%, 96%, 97%, 98% or 99% identical thereto;
CRE0008_GFAP (SEQ ID NO: 28) or a sequence which is at least 60%, 65%, 70%, 75%, 80%, 90%, 95%, 96%, 97%, 98% or 99% identical thereto;
CRE0006_AQP4 (SEQ ID NO: 37) or a sequence which is at least 60%, 65%, 70%, 75%, 80%, 90%, 95%, 96%, 97%, 98% or 99% identical thereto; and
CRE0008_AQP4 (SEQ ID NO: 38) or a sequence which is at least 60%, 65%, 70%, 75%, 80%, 90%, 95%, 96%, 97%, 98% or 99% identical thereto
operably linked to a promoter element selected from:
CRE0003_CEND1_mp (SEQ ID NO: 25) or a sequence which is at least 60%, 65%, 70%, 75%, 80%, 90%, 95%, 96%, 97%, 98% or 99% identical thereto;
CRE0009_SYN1_mp (SEQ ID NO: 26) or a sequence which is at least 60%, 65%, 70%, 75%, 80%, 90%, 95%, 96%, 97%, 98% or 99% identical thereto;
CRE0001_CEND1_mp (SEQ ID NO: 29) or a sequence which is at least 60%, 65%, 70%, 75%, 80%, 90%, 95%, 96%, 97%, 98% or 99% identical thereto;
CRE0002_CEND1_mp (SEQ ID NO: 30) or a sequence which is at least 60%, 65%, 70%, 75%, 80%, 90%, 95%, 96%, 97%, 98% or 99% identical thereto;
CRE0008_SYN1_mp (SEQ ID NO: 31) or a sequence which is at least 60%, 65%, 70%, 75%, 80%, 90%, 95%, 96%, 97%, 98% or 99% identical thereto;
CRE0013_SYN1_mp (SEQ ID NO: 32) or a sequence which is at least 60%, 65%, 70%, 75%, 80%, 90%, 95%, 96%, 97%, 98% or 99% identical thereto;
CRE0015_GAP43_mp (SEQ ID NO: 39) or a sequence which is at least 60%, 65%, 70%, 75%, 80%, 90%, 95%, 96%, 97%, 98% or 99% identical thereto;
CRE0016_Eno2_mp (SEQ ID NO: 40) or a sequence which is at least 60%, 65%, 70%, 75%, 80%, 90%, 95%, 96%, 97%, 98% or 99% identical thereto; and/or
CRE0004_GFAP_mp (SEQ ID NO: 41) or a sequence which is at least 60%, 65%, 70%, 75%, 80%, 90%, 95%, 96%, 97%, 98% or 99% identical thereto.

Suitably, the synthetic NS-specific promoter comprises or consists of:
CRE0001_S100B (SEQ ID NO: 22), or a functional variant thereof, operably linked to CRE0001_CEND1_mp (SEQ ID NO: 29), or a functional variant thereof;
CRE0001_S100B (SEQ ID NO: 22), or a functional variant thereof, operably linked to CRE0013_SYN1_mp (SEQ ID NO: 32), or a functional variant thereof;
CRE0001_S100B (SEQ ID NO: 22), or a functional variant thereof, operably linked to CRE0003_CEND1_mp (SEQ ID NO: 25), or a functional variant thereof;
CRE0001_S100B (SEQ ID NO: 22), or a functional variant thereof, operably linked to CRE0009_SYN1_mp (SEQ ID NO: 26), or a functional variant thereof;
CRE0001_S100B (SEQ ID NO: 22), or a functional variant thereof, operably linked to CRE0002_CEND1_mp (SEQ ID NO: 30), or a functional variant thereof;
CRE0001_S100B (SEQ ID NO: 22), or a functional variant thereof, operably linked to CRE0008_SYN1_mp (SEQ ID NO: 31), or a functional variant thereof;
CRE0001_S100B (SEQ ID NO: 22), or a functional variant thereof, operably linked to CRE0015_GAP43_mp (SEQ ID NO: 39), or a functional variant thereof;
CRE0001_S100B (SEQ ID NO: 22), or a functional variant thereof, operably linked to CRE0016_Eno2_mp (SEQ ID NO: 40), or a functional variant thereof;
CRE0001_S100B (SEQ ID NO: 22), or a functional variant thereof, operably linked to CRE0004_GFAP_mp (SEQ ID NO: 41), or a functional variant thereof;
CRE0007_GFAP (SEQ ID NO: 20), or a functional variant thereof, operably linked to CRE0001_CEND1_mp (SEQ ID NO: 29), or a functional variant thereof;
CRE0007_GFAP (SEQ ID NO: 20), or a functional variant thereof, operably linked to CRE0013_SYN1_mp (SEQ ID NO: 32), or a functional variant thereof;
CRE0007_GFAP (SEQ ID NO: 20), or a functional variant thereof, operably linked to CRE0003_CEND1_mp (SEQ ID NO: 25), or a functional variant thereof;
CRE0007_GFAP (SEQ ID NO: 20), or a functional variant thereof, operably linked to CRE0009_SYN1_mp (SEQ ID NO: 26), or a functional variant thereof;
CRE0007_GFAP (SEQ ID NO: 20), or a functional variant thereof, operably linked to CRE0002_CEND1_mp (SEQ ID NO: 30), or a functional variant thereof;
CRE0007_GFAP (SEQ ID NO: 20), or a functional variant thereof, operably linked to CRE0008_SYN1_mp (SEQ ID NO: 31), or a functional variant thereof;
CRE0007_GFAP (SEQ ID NO: 20), or a functional variant thereof, operably linked to CRE0015_GAP43_mp (SEQ ID NO: 39), or a functional variant thereof;
CRE0007_GFAP (SEQ ID NO: 20), or a functional variant thereof, operably linked to CRE0016_Eno2_mp (SEQ ID NO: 40), or a functional variant thereof;
CRE0007_GFAP (SEQ ID NO: 20), or a functional variant thereof, operably linked to CRE0004_GFAP_mp (SEQ ID NO: 41), or a functional variant thereof;
CRE0009_S100B (SEQ ID NO: 23), or a functional variant thereof, operably linked to CRE0001_CEND1_mp (SEQ ID NO: 29), or a functional variant thereof;
CRE0009_S100B (SEQ ID NO: 23), or a functional variant thereof, operably linked to CRE0013_SYN1_mp (SEQ ID NO: 32), or a functional variant thereof;
CRE0009_S100B (SEQ ID NO: 23), or a functional variant thereof, operably linked to CRE0003_CEND1_mp (SEQ ID NO: 25), or a functional variant thereof;
CRE0009_S100B (SEQ ID NO: 23), or a functional variant thereof, operably linked to CRE0009_SYN1_mp (SEQ ID NO: 26), or a functional variant thereof;
CRE0009_S100B (SEQ ID NO: 23), or a functional variant thereof, operably linked to CRE0002_CEND1_mp (SEQ ID NO: 30), or a functional variant thereof;
CRE0009_S100B (SEQ ID NO: 23), or a functional variant thereof, operably linked to CRE0008_SYN1_mp (SEQ ID NO: 31), or a functional variant thereof;
CRE0009_S100B (SEQ ID NO: 23), or a functional variant thereof, operably linked to CRE0015_GAP43_mp (SEQ ID NO: 39), or a functional variant thereof;
CRE0009_S100B (SEQ ID NO: 23), or a functional variant thereof, operably linked to CRE0016_Eno2_mp (SEQ ID NO: 40), or a functional variant thereof;
CRE0009_S100B (SEQ ID NO: 23), or a functional variant thereof, operably linked to CRE0004_GFAP_mp (SEQ ID NO: 41), or a functional variant thereof;
CRE0002_S100B (SEQ ID NO: 24), or a functional variant thereof, operably linked to CRE0001_CEND1_mp (SEQ ID NO: 29), or a functional variant thereof;
CRE0002_S100B (SEQ ID NO: 24), or a functional variant thereof, operably linked to CRE0013_SYN1_mp (SEQ ID NO: 32), or a functional variant thereof;
CRE0009_S100B (SEQ ID NO: 24), or a functional variant thereof, operably linked to CRE0003_CEND1_mp (SEQ ID NO: 25), or a functional variant thereof;
CRE0002_S100B (SEQ ID NO: 24), or a functional variant thereof, operably linked to CRE0009_SYN1_mp (SEQ ID NO: 26), or a functional variant thereof;
CRE0002_S100B (SEQ ID NO: 24), or a functional variant thereof, operably linked to CRE0002_CEND1_mp (SEQ ID NO: 30), or a functional variant thereof;
CRE0002_S100B (SEQ ID NO: 24), or a functional variant thereof, operably linked to CRE0008_SYN1_mp (SEQ ID NO: 31), or a functional variant thereof;
CRE0002_S100B (SEQ ID NO: 24), or a functional variant thereof, operably linked to CRE0015_GAP43_mp (SEQ ID NO: 39), or a functional variant thereof;
CRE0002_S100B (SEQ ID NO: 24), or a functional variant thereof, operably linked to CRE0016_Eno2 mp (SEQ ID NO: 40), or a functional variant thereof;
CRE0002_S100B (SEQ ID NO: 24), or a functional variant thereof, operably linked to CRE0004_GFAP_mp (SEQ ID NO: 41), or a functional variant thereof;
CRE0005_GFAP (SEQ ID NO: 19), or a functional variant thereof, operably linked to CRE0001_CEND1_mp (SEQ ID NO: 29), or a functional variant thereof;
CRE0005_GFAP (SEQ ID NO: 19), or a functional variant thereof, operably linked to CRE0013_SYN1_mp (SEQ ID NO: 32), or a functional variant thereof;
CRE0005_GFAP (SEQ ID NO: 19), or a functional variant thereof, operably linked to CRE0003_CEND1_mp (SEQ ID NO: 25), or a functional variant thereof;
CRE0005_GFAP (SEQ ID NO: 19), or a functional variant thereof, operably linked to CRE0009_SYN1_mp (SEQ ID NO: 26), or a functional variant thereof;
CRE0005_GFAP (SEQ ID NO: 19), or a functional variant thereof, operably linked to CRE0002_CEND1_mp (SEQ ID NO: 30), or a functional variant thereof;
CRE0005_GFAP (SEQ ID NO: 19), or a functional variant thereof, operably linked to CRE0008_SYN1_mp (SEQ ID NO: 31), or a functional variant thereof;
CRE0005_GFAP (SEQ ID NO: 19), or a functional variant thereof, operably linked to CRE0015_GAP43_mp (SEQ ID NO: 39), or a functional variant thereof;
CRE0005_GFAP (SEQ ID NO: 19), or a functional variant thereof, operably linked to CRE0016_Eno2_mp (SEQ ID NO: 40), or a functional variant thereof;
CRE0005_GFAP (SEQ ID NO: 19), or a functional variant thereof, operably linked to CRE0004_GFAP_mp (SEQ ID NO: 41), or a functional variant thereof;
CRE0012_Arc (SEQ ID NO: 21), or a functional variant thereof, operably linked to CRE0001_CEND1_mp (SEQ ID NO: 29), or a functional variant thereof;
CRE0012_Arc (SEQ ID NO: 21), or a functional variant thereof, operably linked to CRE0013_SYN1_mp (SEQ ID NO: 32), or a functional variant thereof;
CRE0012_Arc (SEQ ID NO: 21), or a functional variant thereof, operably linked to CRE0003_CEND1_mp (SEQ ID NO: 25), or a functional variant thereof;
CRE0012_Arc (SEQ ID NO: 21), or a functional variant thereof, operably linked to CRE0009_SYN1_mp (SEQ ID NO: 26), or a functional variant thereof;
CRE0012_Arc (SEQ ID NO: 21), or a functional variant thereof, operably linked to CRE0002_CEND1_mp (SEQ ID NO: 30), or a functional variant thereof;
CRE0012_Arc (SEQ ID NO: 21), or a functional variant thereof, operably linked to CRE0008_SYN1_mp (SEQ ID NO: 31), or a functional variant thereof;
CRE0012_Arc (SEQ ID NO: 21), or a functional variant thereof, operably linked to CRE0015_GAP43_mp (SEQ ID NO: 39), or a functional variant thereof;
CRE0012_Arc (SEQ ID NO: 21), or a functional variant thereof, operably linked to CRE0016_Eno2 mp (SEQ ID NO: 40), or a functional variant thereof;
CRE0012_Arc (SEQ ID NO: 21), or a functional variant thereof, operably linked to CRE0004_GFAP_mp (SEQ ID NO: 41), or a functional variant thereof;
CRE0006_GFAP (SEQ ID NO: 27), or a functional variant thereof, operably linked to CRE0001_CEND1_mp (SEQ ID NO: 29), or a functional variant thereof;
CRE0006_GFAP (SEQ ID NO: 27), or a functional variant thereof, operably linked to CRE0013_SYN1_mp (SEQ ID NO: 32), or a functional variant thereof;
CRE0006_GFAP (SEQ ID NO: 27), or a functional variant thereof, operably linked to CRE0003_CEND1_mp (SEQ ID NO: 25), or a functional variant thereof;
CRE0006_GFAP (SEQ ID NO: 27), or a functional variant thereof, operably linked to CRE0009_SYN1_mp (SEQ ID NO: 26), or a functional variant thereof;
CRE0006_GFAP (SEQ ID NO: 27), or a functional variant thereof, operably linked to CRE0002_CEND1_mp (SEQ ID NO: 30), or a functional variant thereof;
CRE0006_GFAP (SEQ ID NO: 27), or a functional variant thereof, operably linked to CRE0008_SYN1_mp (SEQ ID NO: 31), or a functional variant thereof;
CRE0006_GFAP (SEQ ID NO: 27), or a functional variant thereof, operably linked to CRE0015_GAP43_mp (SEQ ID NO: 39), or a functional variant thereof;
CRE0006_GFAP (SEQ ID NO: 27), or a functional variant thereof, operably linked to CRE0016_Eno2_mp (SEQ ID NO: 40), or a functional variant thereof;
CRE0006_GFAP (SEQ ID NO: 27), or a functional variant thereof, operably linked to CRE0004_GFAP_mp (SEQ ID NO: 41), or a functional variant thereof;
CRE0008_GFAP (SEQ ID NO: 28), or a functional variant thereof, operably linked to CRE0001_CEND1_mp (SEQ ID NO: 29), or a functional variant thereof;
CRE0008_GFAP (SEQ ID NO: 28), or a functional variant thereof, operably linked to CRE0013_SYN1_mp (SEQ ID NO: 32), or a functional variant thereof;
CRE0008_GFAP (SEQ ID NO: 28), or a functional variant thereof, operably linked to CRE0003_CEND1_mp (SEQ ID NO: 25), or a functional variant thereof;
CRE0008_GFAP (SEQ ID NO: 28), or a functional variant thereof, operably linked to CRE0009_SYN1_mp (SEQ ID NO: 26), or a functional variant thereof;
CRE0008_GFAP (SEQ ID NO: 28), or a functional variant thereof, operably linked to CRE0002_CEND1_mp (SEQ ID NO: 30), or a functional variant thereof;
CRE0008_GFAP (SEQ ID NO: 28), or a functional variant thereof, operably linked to CRE0008_SYN1_mp (SEQ ID NO: 31), or a functional variant thereof;
CRE0008_GFAP (SEQ ID NO: 28), or a functional variant thereof, operably linked to CRE0015_GAP43_mp (SEQ ID NO: 39), or a functional variant thereof;
CRE0008_GFAP (SEQ ID NO: 28), or a functional variant thereof, operably linked to CRE0016_Eno2 mp (SEQ ID NO: 40), or a functional variant thereof;
CRE0008_GFAP (SEQ ID NO: 28), or a functional variant thereof, operably linked to CRE0004_GFAP_mp (SEQ ID NO: 41), or a functional variant thereof;
CRE0006_AQP4 (SEQ ID NO: 37), or a functional variant thereof, operably linked to CRE0001_CEND1_mp (SEQ ID NO: 29), or a functional variant thereof;
CRE0006_AQP4 (SEQ ID NO: 37), or a functional variant thereof, operably linked to CRE0013_SYN1_mp (SEQ ID NO: 32), or a functional variant thereof;
CRE0006_AQP4 (SEQ ID NO: 37), or a functional variant thereof, operably linked to CRE0003_CEND1_mp (SEQ ID NO: 25), or a functional variant thereof;
CRE0006_AQP4 (SEQ ID NO: 37), or a functional variant thereof, operably linked to CRE0009_SYN1_mp (SEQ ID NO: 26), or a functional variant thereof;
CRE0006_AQP4 (SEQ ID NO: 37), or a functional variant thereof, operably linked to CRE0002_CEND1_mp (SEQ ID NO: 30), or a functional variant thereof;
CRE0006_AQP4 (SEQ ID NO: 37), or a functional variant thereof, operably linked to CRE0008_SYN1_mp (SEQ ID NO: 31), or a functional variant thereof;
CRE0006_AQP4 (SEQ ID NO: 37), or a functional variant thereof, operably linked to CRE0015_GAP43_mp (SEQ ID NO: 39), or a functional variant thereof;
CRE0006_AQP4 (SEQ ID NO: 37), or a functional variant thereof, operably linked to CRE0016_Eno2_mp (SEQ ID NO: 40), or a functional variant thereof;
CRE0006_AQP4 (SEQ ID NO: 37), or a functional variant thereof, operably linked to CRE0004_GFAP_mp (SEQ ID NO: 41), or a functional variant thereof;
CRE0008_AQP4 (SEQ ID NO: 38), or a functional variant thereof, operably linked to CRE0001_CEND1_mp (SEQ ID NO: 29), or a functional variant thereof;
CRE0008_AQP4 (SEQ ID NO: 38), or a functional variant thereof, operably linked to CRE0013_SYN1_mp (SEQ ID NO: 32), or a functional variant thereof;
CRE0008_AQP4 (SEQ ID NO: 38), or a functional variant thereof, operably linked to CRE0003_CEND1_mp (SEQ ID NO: 25), or a functional variant thereof;
CRE0008_AQP4 (SEQ ID NO: 38), or a functional variant thereof, operably linked to CRE0009_SYN1_mp (SEQ ID NO: 26), or a functional variant thereof;
CRE0008_AQP4 (SEQ ID NO: 38), or a functional variant thereof, operably linked to CRE0002_CEND1_mp (SEQ ID NO: 30), or a functional variant thereof;
CRE0008_AQP4 (SEQ ID NO: 38), or a functional variant thereof, operably linked to CRE0008_SYN1_mp (SEQ ID NO: 31), or a functional variant thereof;
CRE0008_AQP4 (SEQ ID NO: 38), or a functional variant thereof, operably linked to CRE0015_GAP43_mp (SEQ ID NO: 39), or a functional variant thereof;
CRE0008_AQP4 (SEQ ID NO: 38), or a functional variant thereof, operably linked to CRE0016_Eno2 mp (SEQ ID NO: 40), or a functional variant thereof; or
CRE0008_AQP4 (SEQ ID NO: 38), or a functional variant thereof, operably linked to CRE0004_GFAP_mp (SEQ ID NO: 41), or a functional variant thereof.

Suitably, there is provided a synthetic NS-specific promoter comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof;
CRE0009_S100B (SEQ ID NO: 23) or a functional variant thereof;
CRE0006_AQP4 (SEQ ID NO: 37) or a functional variant thereof; and
CRE0008_AQP4 (SEQ ID NO: 38) or a functional variant thereof
operably linked to a promoter element, optionally wherein the promoter element is selected from:
CRE0001_CEND1_mp (SEQ ID NO: 29) or a functional variant thereof;
CRE0013_SYN1_mp (SEQ ID NO: 32) or a functional variant thereof;
CRE0004_GFAP_mp (SEQ ID NO: 41) or a functional variant thereof;
CRE0015_GAP43_mp (SEQ ID NO: 40) or a functional variant thereof; and/or
CRE0016_Eno2_mp (SEQ ID NO: 39) or a functional variant thereof.

Suitably, there is provided a synthetic NS-specific promoter comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof;
CRE0009_S100B (SEQ ID NO: 23) or a functional variant thereof;
CRE0006_AQP4 (SEQ ID NO: 37) or a functional variant thereof; and
CRE0008_AQP4 (SEQ ID NO: 38) or a functional variant thereof
operably linked to a promoter element selected from:
CRE0001_CEND1_mp (SEQ ID NO: 29) or a functional variant thereof;
CRE0013_SYN1_mp (SEQ ID NO: 32) or a functional variant thereof;
CRE0004_GFAP_mp (SEQ ID NO: 41) or a functional variant thereof;
CRE0015_GAP43_mp (SEQ ID NO: 40) or a functional variant thereof; and/or
CRE0016_Eno2_mp (SEQ ID NO: 39) or a functional variant thereof.

Functional variants are defined herein below. Suitably, the synthetic NS-specific promoter may comprise a sequence which is at least 60%, 65%, 70%, 75%, 80%, 90%, 95%, 96%, 97%, 98% or 99% identical to any one of SEQ ID NOs: 20, 22, 23, 37, 38, 29, 32, 41, 40, 39. Suitably the synthetic NS-specific promoter may comprise a sequence which is at least 60%, 65%, 70%, 75%, 80%, 90%, 95%, 96%, 97%, 98% or 99% identical to any one of SEQ ID NO: 20, 22, 37, 38 or 23, operably linked to a sequence which is at least 60%, 65%, 70%, 75%, 80%, 90%, 95%, 96%, 97%, 98% or 99% identical to any one of SEQ ID NOs: 29, 41, 40, 39 or 32.

In one embodiment, there is provided a synthetic nervous system (NS)-specific promoter comprising or consisting of at least one of the following cis-regulatory elements (CREs):
CRE0001_S100B (SEQ ID NO: 22) or a sequence which is at least 60%, 65%, 70%, 75%, 80%, 90%, 95%, 96%, 97%, 98% or 99% identical thereto;
CRE0007_GFAP (SEQ ID NO: 20) or a sequence which is at least 60%, 65%, 70%, 75%, 80%, 90%, 95%, 96%, 97%, 98% or 99% identical thereto;
CRE0009_S100B (SEQ ID NO: 23) or a sequence which is at least 60%, 65%, 70%, 75%, 80%, 90%, 95%, 96%, 97%, 98% or 99% identical thereto;
CRE0006_AQP4 (SEQ ID NO: 37) or a sequence which is at least 60%, 65%, 70%, 75%, 80%, 90%, 95%, 96%, 97%, 98% or 99% identical thereto;
CRE0008_AQP4 (SEQ ID NO: 38) or a sequence which is at least 60%, 65%, 70%, 75%, 80%, 90%, 95%, 96%, 97%, 98% or 99% identical thereto
operably linked to a promoter element selected from:
CRE0001_CEND1_mp (SEQ ID NO: 29) or a sequence which is at least 60%, 65%, 70%, 75%, 80%, 90%, 95%, 96%, 97%, 98% or 99% identical thereto;
CRE0013_SYN1_mp (SEQ ID NO: 32) or a sequence which is at least 60%, 65%, 70%, 75%, 80%, 90%, 95%, 96%, 97%, 98% or 99% identical thereto;
CRE0004_GFAP_mp (SEQ ID NO: 41) or a sequence which is at least 60%, 65%, 70%, 75%, 80%, 90%, 95%, 96%, 97%, 98% or 99% identical thereto;
CRE0015_GAP43_mp (SEQ ID NO: 40) or a sequence which is at least 60%, 65%, 70%, 75%, 80%, 90%, 95%, 96%, 97%, 98% or 99% identical thereto; and
CRE0016_Eno2_mp (SEQ ID NO: 39) or a sequence which is at least 60%, 65%, 70%, 75%, 80%, 90%, 95%, 96%, 97%, 98% or 99% identical thereto.

Suitably, the synthetic NS-specific promoter comprises or consists of:
CRE0001_S100B (SEQ ID NO: 22), or a functional variant thereof, operably linked to CRE0001_CEND1_mp (SEQ ID NO: 29), or a functional variant thereof;
CRE0001_S100B (SEQ ID NO: 22), or a functional variant thereof, operably linked to CRE0013_SYN1_mp (SEQ ID NO: 32), or a functional variant thereof;
CRE0001_S100B (SEQ ID NO: 22), or a functional variant thereof, operably linked to CRE0004_GFAP_mp (SEQ ID NO: 41), or a functional variant thereof;
CRE0001_S100B (SEQ ID NO: 22), or a functional variant thereof, operably linked to CRE0015_GAP43_mp (SEQ ID NO: 40), or a functional variant thereof;
CRE0001_S100B (SEQ ID NO: 22), or a functional variant thereof, operably linked to CRE0016_Eno2_mp (SEQ ID NO: 39), or a functional variant thereof;
CRE0007_GFAP (SEQ ID NO: 20), or a functional variant thereof, operably linked to CRE0001_CEND1_mp (SEQ ID NO: 29), or a functional variant thereof;
CRE0007_GFAP (SEQ ID NO: 20), or a functional variant thereof, operably linked to CRE0013_SYN1_mp (SEQ ID NO: 32), or a functional variant thereof;
CRE0007_GFAP (SEQ ID NO: 20), or a functional variant thereof, operably linked to CRE0004_GFAP_mp (SEQ ID NO: 41), or a functional variant thereof;
CRE0007_GFAP (SEQ ID NO: 20), or a functional variant thereof, operably linked to CRE0015_GAP43_mp (SEQ ID NO: 40), or a functional variant thereof;
CRE0007_GFAP (SEQ ID NO: 20), or a functional variant thereof, operably linked to CRE0016_Eno2_mp (SEQ ID NO: 39), or a functional variant thereof;
CRE0009_S100B (SEQ ID NO: 23), or a functional variant thereof, operably linked to CRE0001_CEND1_mp (SEQ ID NO: 29), or a functional variant thereof;
CRE0009_S100B (SEQ ID NO: 23), or a functional variant thereof, operably linked to CRE0013_SYN1_mp (SEQ ID NO: 32), or a functional variant thereof;
CRE0009_S100B (SEQ ID NO: 23), or a functional variant thereof, operably linked to CRE0004_GFAP_mp (SEQ ID NO: 41), or a functional variant thereof;
CRE0009_S100B (SEQ ID NO: 23), or a functional variant thereof, operably linked to CRE0015_GAP43_mp (SEQ ID NO: 40), or a functional variant thereof;
CRE0009_S100B (SEQ ID NO: 23), or a functional variant thereof, operably linked to CRE0016_Eno2_mp (SEQ ID NO: 39), or a functional variant thereof;
CRE0006_AQP4 (SEQ ID NO: 37), or a functional variant thereof, operably linked to CRE0001_CEND1_mp (SEQ ID NO: 29), or a functional variant thereof;
CRE0006_AQP4 (SEQ ID NO: 37), or a functional variant thereof, operably linked to CRE0013_SYN1_mp (SEQ ID NO: 32), or a functional variant thereof;
CRE0006_AQP4 (SEQ ID NO: 37), or a functional variant thereof, operably linked to CRE0004_GFAP_mp (SEQ ID NO: 41), or a functional variant thereof;
CRE0006_AQP4 (SEQ ID NO: 37), or a functional variant thereof, operably linked to CRE0015_GAP43_mp (SEQ ID NO: 40), or a functional variant thereof;
CRE0006_AQP4 (SEQ ID NO: 37), or a functional variant thereof, operably linked to CRE0016_Eno2_mp (SEQ ID NO: 39), or a functional variant thereof;
CRE0008_AQP4 (SEQ ID NO: 38), or a functional variant thereof, operably linked to CRE0001_CEND1 mp (SEQ ID NO: 29), or a functional variant thereof;
CRE0008_AQP4 (SEQ ID NO: 38), or a functional variant thereof, operably linked to CRE0013_SYN1_mp (SEQ ID NO: 32), or a functional variant thereof;
CRE0008_AQP4 (SEQ ID NO: 38), or a functional variant thereof, operably linked to CRE0004_GFAP_mp (SEQ ID NO: 41), or a functional variant thereof;
CRE0008_AQP4 (SEQ ID NO: 38), or a functional variant thereof, operably linked to CRE0015_GAP43_mp (SEQ ID NO: 40), or a functional variant thereof; or
CRE0008_AQP4 (SEQ ID NO: 38), or a functional variant thereof, operably linked to CRE0016_Eno2_mp (SEQ ID NO: 39), or a functional variant thereof.

Suitably, there is provided a synthetic CNS-specific promoter comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof; and
CRE0009_S100B (SEQ ID NO: 23) or a functional variant thereof
operably linked to a promoter element, optionally wherein the promoter element is selected from:
CRE0001_CEND1_mp (SEQ ID NO: 29) or a functional variant thereof; and/or
CRE0013_SYN1_mp (SEQ ID NO: 32) or a functional variant thereof.

Suitably, there is provided a synthetic NS-specific promoter comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof; and
CRE0009_S100B (SEQ ID NO: 23) or a functional variant thereof
operably linked to a promoter element selected from:
CRE0001_CEND1_mp (SEQ ID NO: 29) or a functional variant thereof; and/or
CRE0013_SYN1_mp (SEQ ID NO: 32) or a functional variant thereof.

Functional variants are defined herein below. Suitably, the synthetic NS-specific promoter may comprise a sequence which is at least 60%, 65%, 70%, 75%, 80%, 90%, 95%, 96%, 97%, 98% or 99% identical to any one of SEQ ID NOs: 20, 22, 23, 29, 32. Suitably the synthetic NS-specific promoter may comprise a sequence which is at least 60%, 65%, 70%, 75%, 80%, 90%, 95%, 96%, 97%, 98% or 99% identical to any one of SEQ ID NO: 20, 22 or 23, operably linked to a sequence which is at least 60%, 65%, 70%, 75%, 80%, 90%, 95%, 96%, 97%, 98% or 99% identical to any one of SEQ ID NOs: 29 or 32.

In one embodiment, there is provided a synthetic nervous system (NS)-specific promoter comprising or consisting of at least one of the following cis-regulatory elements (CREs):
CRE0001_S100B (SEQ ID NO: 22) or a sequence which is at least 60%, 65%, 70%, 75%, 80%, 90%, 95%, 96%, 97%, 98% or 99% identical thereto;
CRE0007_GFAP (SEQ ID NO: 20) or a sequence which is at least 60%, 65%, 70%, 75%, 80%, 90%, 95%, 96%, 97%, 98% or 99% identical thereto;
CRE0009_S100B (SEQ ID NO: 23) or a sequence which is at least 60%, 65%, 70%, 75%, 80%, 90%, 95%, 96%, 97%, 98% or 99% identical thereto;
operably linked to a promoter element selected from:
CRE0001_CEND1_mp (SEQ ID NO: 29) or a sequence which is at least 60%, 65%, 70%, 75%, 80%, 90%, 95%, 96%, 97%, 98% or 99% identical thereto;
CRE0013_SYN1_mp (SEQ ID NO: 32) or a sequence which is at least 60%, 65%, 70%, 75%, 80%, 90%, 95%, 96%, 97%, 98% or 99% identical thereto.

Suitably, the synthetic NS-specific promoter comprises or consists of:
CRE0001_S100B (SEQ ID NO: 22), or a functional variant thereof, operably linked to CRE0001_CEND1_mp (SEQ ID NO: 29), or a functional variant thereof;
CRE0001_S100B (SEQ ID NO: 22), or a functional variant thereof, operably linked to CRE0013_SYN1_mp (SEQ ID NO: 32), or a functional variant thereof;
CRE0007_GFAP (SEQ ID NO: 20), or a functional variant thereof, operably linked to CRE0001_CEND1_mp (SEQ ID NO: 29), or a functional variant thereof;
CRE0007_GFAP (SEQ ID NO: 20), or a functional variant thereof, operably linked to CRE0013_SYN1_mp (SEQ ID NO: 32), or a functional variant thereof;
CRE0009_S100B (SEQ ID NO: 23), or a functional variant thereof, operably linked to CRE0001_CEND1_mp (SEQ ID NO: 29), or a functional variant thereof; or
CRE0009_S100B (SEQ ID NO: 23), or a functional variant thereof, operably linked to CRE0013_SYN1_mp (SEQ ID NO: 32), or a functional variant thereof.

Suitably, there is provided a synthetic NS-specific promoter comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof;
CRE0009_S100B (SEQ ID NO: 23) or a functional variant thereof;
CRE0006_AQP4 (SEQ ID NO: 37) or functional variant thereof; and
CRE0008_AQP4 (SEQ ID NO: 38) or functional variant thereof operably linked to a promoter element, optionally wherein the promoter element is selected from:
CRE0003_CEND1_mp (SEQ ID NO: 25) or a functional variant thereof;
CRE0009_SYN1_mp (SEQ ID NO: 26) or a functional variant thereof;
CRE0015_GAP43_mp (SEQ ID NO: 39) or a functional variant thereof;
CRE0016_Eno2_mp (SEQ ID NO: 40) or a functional variant thereof; and/or
CRE0004_GFAP_mp (SEQ ID NO: 41) or a functional variant thereof.

Suitably, there is provided a synthetic NS-specific promoter comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof;
CRE0009_S100B (SEQ ID NO: 23) or a functional variant thereof;
CRE0006_AQP4 (SEQ ID NO: 37) or functional variant thereof; and
CRE0008_AQP4 (SEQ ID NO: 38) or functional variant thereof
operably linked to a promoter element selected from:
CRE0003_CEND1_mp (SEQ ID NO: 25) or a functional variant thereof;
CRE0009_SYN1_mp (SEQ ID NO: 26) or a functional variant thereof;
CRE0015_GAP43_mp (SEQ ID NO: 39) or a functional variant thereof;
CRE0016_Eno2_mp (SEQ ID NO: 40) or a functional variant thereof; and/or
CRE0004_GFAP_mp (SEQ ID NO: 41) or a functional variant thereof.

Functional variants are defined herein below. Suitably, the synthetic NS-specific promoter may comprise a sequence which is at least 60%, 65%, 70%, 75%, 80%, 90%, 95%, 96%, 97%, 98% or 99% identical to any one of SEQ ID NOs: 20, 22, 23, 37, 38, 25, 26, 39, 40, 41. Suitably the synthetic NS-specific promoter may comprise a sequence which is at least 60%, 65%, 70%, 75%, 80%, 90%, 95%, 96%, 97%, 98% or 99% identical to any one of SEQ ID NO: 20, 22, 37, 38 or 23, operably linked to a sequence which is at least 60%, 65%, 70%, 75%, 80%, 90%, 95%, 96%, 97%, 98% or 99% identical to any one of SEQ ID NOs: 25, 39, 40, 41 or 26.

In one embodiment, there is provided a synthetic nervous system (NS)-specific promoter comprising or consisting of at least one of the following cis-regulatory elements (CREs):
CRE0001_S100B (SEQ ID NO: 22) or a sequence which is at least 60%, 65%, 70%, 75%, 80%, 90%, 95%, 96%, 97%, 98% or 99% identical thereto;
CRE0007_GFAP (SEQ ID NO: 20) or a sequence which is at least 60%, 65%, 70%, 75%, 80%, 90%, 95%, 96%, 97%, 98% or 99% identical thereto;
CRE0009_S100B (SEQ ID NO: 23) or a sequence which is at least 60%, 65%, 70%, 75%, 80%, 90%, 95%, 96%, 97%, 98% or 99% identical thereto;
CRE0006_AQP4 (SEQ ID NO: 37) or a sequence which is at least 60%, 65%, 70%, 75%, 80%, 90%, 95%, 96%, 97%, 98% or 99% identical thereto; and
CRE0008_AQP4 (SEQ ID NO: 38) or a sequence which is at least 60%, 65%, 70%, 75%, 80%, 90%, 95%, 96%, 97%, 98% or 99% identical thereto;
operably linked to a promoter element selected from:
CRE0003_CEND1_mp (SEQ ID NO: 25) or a sequence which is at least 60%, 65%, 70%, 75%, 80%, 90%, 95%, 96%, 97%, 98% or 99% identical thereto;
CRE0009_SYN1_mp (SEQ ID NO: 26) or a sequence which is at least 60%, 65%, 70%, 75%, 80%, 90%, 95%, 96%, 97%, 98% or 99% identical thereto;
CRE0015_GAP43_mp (SEQ ID NO: 39) or a sequence which is at least 60%, 65%, 70%, 75%, 80%, 90%, 95%, 96%, 97%, 98% or 99% identical thereto;
CRE0016_Eno2_mp (SEQ ID NO: 40) or a sequence which is at least 60%, 65%, 70%, 75%, 80%, 90%, 95%, 96%, 97%, 98% or 99% identical thereto; and/or
CRE0004_GFAP_mp (SEQ ID NO: 41) or a sequence which is at least 60%, 65%, 70%, 75%, 80%, 90%, 95%, 96%, 97%, 98% or 99% identical thereto.

Suitably, the synthetic NS-specific promoter comprises or consists of:
CRE0001_S100B (SEQ ID NO: 22), or a functional variant thereof, operably linked to CRE0003_CEND1_mp (SEQ ID NO: 25), or a functional variant thereof;
CRE0001_S100B (SEQ ID NO: 22), or a functional variant thereof, operably linked to CRE0009_SYN1_mp (SEQ ID NO: 26), or a functional variant thereof;
CRE0001_S100B (SEQ ID NO: 22), or a functional variant thereof, operably linked to CRE0015_GAP43_mp (SEQ ID NO: 39), or a functional variant thereof;
CRE0001_S100B (SEQ ID NO: 22), or a functional variant thereof, operably linked to CRE0016_Eno2_mp (SEQ ID NO: 40), or a functional variant thereof;
CRE0001_S100B (SEQ ID NO: 22), or a functional variant thereof, operably linked to CRE0004_GFAP_mp (SEQ ID NO: 41), or a functional variant thereof;
CRE0007_GFAP (SEQ ID NO: 20), or a functional variant thereof, operably linked to CRE0003_CEND1_mp (SEQ ID NO: 25), or a functional variant thereof;
CRE0007_GFAP (SEQ ID NO: 20), or a functional variant thereof, operably linked to CRE0009_SYN1 mp (SEQ ID NO: 26), or a functional variant thereof;
CRE0007_GFAP (SEQ ID NO: 20), or a functional variant thereof, operably linked to CRE0015_GAP43_mp (SEQ ID NO: 39), or a functional variant thereof;
CRE0007_GFAP (SEQ ID NO: 20), or a functional variant thereof, operably linked to CRE0016_Eno2_mp (SEQ ID NO: 40), or a functional variant thereof;
CRE0007_GFAP (SEQ ID NO: 20), or a functional variant thereof, operably linked to CRE0004_GFAP_mp (SEQ ID NO: 41), or a functional variant thereof;
CRE0009_S100B (SEQ ID NO: 23), or a functional variant thereof, operably linked to CRE0003_CEND1_mp (SEQ ID NO: 25), or a functional variant thereof;
CRE0009_S100B (SEQ ID NO: 23), or a functional variant thereof, operably linked to CRE0009_SYN1_mp (SEQ ID NO: 26), or a functional variant thereof;
CRE0009_S100B (SEQ ID NO: 23), or a functional variant thereof, operably linked to CRE0015_GAP43_mp (SEQ ID NO: 39), or a functional variant thereof;
CRE0009_S100B (SEQ ID NO: 23), or a functional variant thereof, operably linked to CRE0016_Eno2_mp (SEQ ID NO: 40), or a functional variant thereof;
CRE0009_S100B (SEQ ID NO: 23), or a functional variant thereof, operably linked to CRE0004_GFAP_mp (SEQ ID NO: 41), or a functional variant thereof;
CRE0006_AQP4 (SEQ ID NO: 37), or a functional variant thereof, operably linked to CRE0003_CEND1_mp (SEQ ID NO: 25), or a functional variant thereof;
CRE0006_AQP4 (SEQ ID NO: 37), or a functional variant thereof, operably linked to CRE0009_SYN1_mp (SEQ ID NO: 26), or a functional variant thereof;
CRE0006_AQP4 (SEQ ID NO: 37), or a functional variant thereof, operably linked to CRE0015_GAP43_mp (SEQ ID NO: 39), or a functional variant thereof;
CRE0006_AQP4 (SEQ ID NO: 37), or a functional variant thereof, operably linked to CRE0016_Eno2_mp (SEQ ID NO: 40), or a functional variant thereof;
CRE0006_AQP4 (SEQ ID NO: 37), or a functional variant thereof, operably linked to CRE0004_GFAP_mp (SEQ ID NO: 41), or a functional variant thereof;
CRE0008_AQP4 (SEQ ID NO: 38), or a functional variant thereof, operably linked to CRE0003_CEND1_mp (SEQ ID NO: 25), or a functional variant thereof;
CRE0008_AQP4 (SEQ ID NO: 38), or a functional variant thereof, operably linked to CRE0009_SYN1_mp (SEQ ID NO: 26), or a functional variant thereof;
CRE0008_AQP4 (SEQ ID NO: 38), or a functional variant thereof, operably linked to CRE0015_GAP43_mp (SEQ ID NO: 39), or a functional variant thereof;
CRE0008_AQP4 (SEQ ID NO: 38), or a functional variant thereof, operably linked to CRE0016_Eno2_mp (SEQ ID NO: 40), or a functional variant thereof;
CRE0008_AQP4 (SEQ ID NO: 38), or a functional variant thereof, operably linked to CRE0004_GFAP_mp (SEQ ID NO: 41), or a functional variant thereof;

Suitably, there is provided a synthetic nervous system (NS)-specific promoter comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof; and
CRE0009_S100B (SEQ ID NO: 23) or a functional variant thereof
operably linked to a promoter element, optionally wherein the promoter element is selected from:
CRE0003_CEND1_mp (SEQ ID NO: 25) or a functional variant thereof; and/or
CRE0009_SYN1_mp (SEQ ID NO: 26) or a functional variant thereof.

Suitably, there is provided a synthetic nervous system (NS)-specific promoter comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof; and
CRE0009_S100B (SEQ ID NO: 23) or a functional variant thereof
operably linked to a promoter element selected from:
CRE0003_CEND1_mp (SEQ ID NO: 25) or a functional variant thereof; and/or
CRE0009_SYN1_mp (SEQ ID NO: 26) or a functional variant thereof.

Functional variants are defined hereinbelow. Suitably, the synthetic NS-specific promoter may comprise a sequence which is at least 60%, 65%, 70%, 75%, 80%, 90%, 95%, 96%, 97%, 98% or 99% identical to any one of SEQ ID NOs: 20, 22, 23, 25-26. Suitably the synthetic NS-specific promoter may comprise a sequence which is at least 60%, 65%, 70%, 75%, 80%, 90%, 95%, 96%, 97%, 98% or 99% identical to any one of SEQ ID NO: 20, 22 or 23, operably linked to a sequence which is at least 60%, 65%, 70%, 75%, 80%, 90%, 95%, 96%, 97%, 98% or 99% identical to any one of SEQ ID NOs: 25 or 26.

In one embodiment, there is provided a synthetic nervous system (NS)-specific promoter comprising or consisting of at least one of the following cis-regulatory elements (CREs):
CRE0001_S100B (SEQ ID NO: 22) or a sequence which is at least 60%, 65%, 70%, 75%, 80%, 90%, 95%, 96%, 97%, 98% or 99% identical thereto;
CRE0007_GFAP (SEQ ID NO: 20) or a sequence which is at least 60%, 65%, 70%, 75%, 80%, 90%, 95%, 96%, 97%, 98% or 99% identical thereto;
CRE0009_S100B (SEQ ID NO: 23) or a sequence which is at least 60%, 65%, 70%, 75%, 80%, 90%, 95%, 96%, 97%, 98% or 99% identical thereto;
operably linked to a promoter element selected from:
CRE0003_CEND1_mp (SEQ ID NO: 25) or a sequence which is at least 60%, 65%, 70%, 75%, 80%, 90%, 95%, 96%, 97%, 98% or 99% identical thereto; and/or
CRE0009_SYN1_mp (SEQ ID NO: 26) or a sequence which is at least 60%, 65%, 70%, 75%, 80%, 90%, 95%, 96%, 97%, 98% or 99% identical thereto.

Suitably, the synthetic NS-specific promoter comprises or consists of:
CRE0001_S100B (SEQ ID NO: 22), or a functional variant thereof, operably linked to CRE0003_CEND1_mp (SEQ ID NO: 25), or a functional variant thereof;
CRE0001_S100B (SEQ ID NO: 22), or a functional variant thereof, operably linked to CRE0009_SYN1_mp (SEQ ID NO: 26), or a functional variant thereof;
CRE0007_GFAP (SEQ ID NO: 20), or a functional variant thereof, operably linked to CRE0003_CEND1_mp (SEQ ID NO: 25), or a functional variant thereof;
CRE0007_GFAP (SEQ ID NO: 20), or a functional variant thereof, operably linked to CRE0009_SYN1_mp (SEQ ID NO: 26), or a functional variant thereof;
CRE0009_S100B (SEQ ID NO: 23), or a functional variant thereof, operably linked to CRE0003_CEND1_mp (SEQ ID NO: 25), or a functional variant thereof; or
CRE0009_S100B (SEQ ID NO: 23), or a functional variant thereof, operably linked to CRE0009_SYN1_mp (SEQ ID NO: 26), or a functional variant thereof.

A NS-specific promoter can be expressed in other cells. However, it has a higher degree of expression in the NS cells such as neuronal cells in the brain, spinal cord and peripheral nervous system, as well as non-neuronal cells, interneurons, or neuronal supporting cells located in the brain, spinal cord and peripheral nervous system. For example, a NS-specific promoter expresses a gene at least 25%, or at least 35%, or at least 45%, or at least 55%, or at least 65%, or at least 75%, or at least 80%, or at least 85%, or at least 90%, or at least 95%, or any integer between 25%-95% higher in cells located in the NS, including neuronal and non-neuronal cells and interneurons located in the brain, spinal cord and the peripheral nervous system as compared to cells located outside the CNS and/or PNS.

Suitably, the synthetic NS-specific promoter may comprise one or more additional CREs or functional variants thereof. The CREs may be CREs according to the present invention (any one of SEQ ID NOs: 19-24, 27, 28, 37, 38), functional variants thereof, or other CREs. Suitably, when the one or more additional CREs are not CREs according to the present invention, the one or more additional CRE may be selected from any CRE known in the art. For example, in one embodiment, the one or more additional CREs may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. In some preferred embodiments, the synthetic NS-specific promoter may additionally comprise CRE0012_Arc (SEQ ID NO: 21), or a functional variant thereof.

Suitably, the synthetic NS-specific promoter comprises or consists of:
CRE0001_S100B (SEQ ID NO: 22), or a functional variant thereof, and CRE0012_Arc (SEQ ID NO: 21), or a functional variant thereof, operably linked to CRE0001_CEND1_mp (SEQ ID NO: 29), or a functional variant thereof;
CRE0001_S100B (SEQ ID NO: 22), or a functional variant thereof, and CRE0012_Arc (SEQ ID NO: 21), or a functional variant thereof, operably linked to CRE0013_SYN1_mp (SEQ ID NO: 32), or a functional variant thereof;
CRE0007_GFAP (SEQ ID NO: 20), or a functional variant thereof, and CRE0012_Arc (SEQ ID NO: 21), or a functional variant thereof, operably linked to CRE0001_CEND1_mp (SEQ ID NO: 29), or a functional variant thereof;
CRE0007_GFAP (SEQ ID NO: 20), or a functional variant thereof, and CRE0012_Arc (SEQ ID NO: 21), or a functional variant thereof, operably linked to CRE0013_SYN1_mp (SEQ ID NO: 32), or a functional variant thereof;
CRE0009_S100B (SEQ ID NO: 23), or a functional variant thereof, and CRE0012_Arc (SEQ ID NO: 21), or a functional variant thereof, operably linked to CRE0001_CEND1_mp (SEQ ID NO: 29), or a functional variant thereof; or
CRE0009_S100B (SEQ ID NO: 23), or a functional variant thereof, and CRE0012_Arc (SEQ ID NO: 21), or a functional variant thereof, operably linked to CRE0013_SYN1_mp (SEQ ID NO: 32), or a functional variant thereof.
CRE0001_S100B (SEQ ID NO: 22), or a functional variant thereof, and CRE0012_Arc (SEQ ID NO: 21), or a functional variant thereof, operably linked to CRE0003_CEND1_mp (SEQ ID NO: 25), or a functional variant thereof;
CRE0001_S100B (SEQ ID NO: 22), or a functional variant thereof, and CRE0012_Arc (SEQ ID NO: 21), or a functional variant thereof, operably linked to CRE0009_SYN1_mp (SEQ ID NO: 26), or a functional variant thereof;
CRE0007_GFAP (SEQ ID NO: 20), or a functional variant thereof, and CRE0012_Arc (SEQ ID NO: 21), or a functional variant thereof, operably linked to CRE0003_CEND1 mp (SEQ ID NO: 25), or a functional variant thereof;
CRE0007_GFAP (SEQ ID NO: 20), or a functional variant thereof, and CRE0012_Arc (SEQ ID NO: 21), or a functional variant thereof, operably linked to CRE0009_SYN1_mp (SEQ ID NO: 26), or a functional variant thereof;
CRE0009_S100B (SEQ ID NO: 23), or a functional variant thereof, and CRE0012_Arc (SEQ ID NO: 21), or a functional variant thereof, operably linked to CRE0003_CEND1_mp (SEQ ID NO: 25), or a functional variant thereof; or
CRE0009_S100B (SEQ ID NO: 23), or a functional variant thereof, and CRE0012_Arc (SEQ ID NO: 21), or a functional variant thereof, operably linked to CRE0009_SYN1_mp (SEQ ID NO: 26), or a functional variant thereof.
CRE0001_S100B (SEQ ID NO: 22), or a functional variant thereof, and CRE0012_Arc (SEQ ID NO: 21), or a functional variant thereof, operably linked to CRE0015_GAP43_mp (SEQ ID NO: 39), or a functional variant thereof;
CRE0001_S100B (SEQ ID NO: 22), or a functional variant thereof, and CRE0012_Arc (SEQ ID NO: 21), or a functional variant thereof, operably linked to CRE0016_Eno2_mp (SEQ ID NO: 40), or a functional variant thereof;
CRE0001_S100B (SEQ ID NO: 22), or a functional variant thereof, and CRE0012_Arc (SEQ ID NO: 21), or a functional variant thereof, operably linked to CRE0004_GFAP_mp (SEQ ID NO: 41), or a functional variant thereof;
CRE0007_GFAP (SEQ ID NO: 20), or a functional variant thereof, and CRE0012_Arc (SEQ ID NO: 21), or a functional variant thereof, operably linked to CRE0015_GAP43_mp (SEQ ID NO: 39), or a functional variant thereof;
CRE0007_GFAP (SEQ ID NO: 20), or a functional variant thereof, and CRE0012_Arc (SEQ ID NO: 21), or a functional variant thereof, operably linked to CRE0016_Eno2_mp (SEQ ID NO: 40), or a functional variant thereof;
CRE0007_GFAP (SEQ ID NO: 20), or a functional variant thereof, and CRE0012_Arc (SEQ ID NO: 21), or a functional variant thereof, operably linked to CRE0004_GFAP_mp (SEQ ID NO: 41), or a functional variant thereof;
CRE0009_S100B (SEQ ID NO: 23), or a functional variant thereof, and CRE0012_Arc (SEQ ID NO: 21), or a functional variant thereof, operably linked to CRE0015_GAP43_mp (SEQ ID NO: 39), or a functional variant thereof;
CRE0009_S100B (SEQ ID NO: 23), or a functional variant thereof, and CRE0012_Arc (SEQ ID NO: 21), or a functional variant thereof, operably linked to CRE0016_Eno2_mp (SEQ ID NO: 40), or a functional variant thereof;
CRE0009_S100B (SEQ ID NO: 23), or a functional variant thereof, and CRE0012_Arc (SEQ ID NO: 21), or a functional variant thereof, operably linked to CRE0004_GFAP_mp (SEQ ID NO: 41), or a functional variant thereof;
CRE0001_S100B (SEQ ID NO: 22), or a functional variant thereof, and CRE0012_Arc (SEQ ID NO: 21), or a functional variant thereof, operably linked to CRE0015_GAP43_mp (SEQ ID NO: 39), or a functional variant thereof;
CRE0001_S100B (SEQ ID NO: 22), or a functional variant thereof, and CRE0012_Arc (SEQ ID NO: 21), or a functional variant thereof, operably linked to CRE0016_Eno2_mp (SEQ ID NO: 40), or a functional variant thereof;
CRE0001_S100B (SEQ ID NO: 22), or a functional variant thereof, and CRE0012_Arc (SEQ ID NO: 21), or a functional variant thereof, operably linked to CRE0004_GFAP_mp (SEQ ID NO: 41), or a functional variant thereof;
CRE0007_GFAP (SEQ ID NO: 20), or a functional variant thereof, and CRE0012_Arc (SEQ ID NO: 21), or a functional variant thereof, operably linked to CRE0015_GAP43_mp (SEQ ID NO: 39), or a functional variant thereof;
CRE0007_GFAP (SEQ ID NO: 20), or a functional variant thereof, and CRE0012_Arc (SEQ ID NO: 21), or a functional variant thereof, operably linked to CRE0016_Eno2_mp (SEQ ID NO: 40), or a functional variant thereof;
CRE0007_GFAP (SEQ ID NO: 20), or a functional variant thereof, and CRE0012_Arc (SEQ ID NO: 21), or a functional variant thereof, operably linked to CRE0004_GFAP_mp (SEQ ID NO: 41), or a functional variant thereof;
CRE0009_S100B (SEQ ID NO: 23), or a functional variant thereof, and CRE0012_Arc (SEQ ID NO: 21), or a functional variant thereof, operably linked to CRE0015_GAP43_mp (SEQ ID NO: 39), or a functional variant thereof;
CRE0009_S100B (SEQ ID NO: 23), or a functional variant thereof, and CRE0012_Arc (SEQ ID NO: 21), or a functional variant thereof, operably linked to CRE0016_Eno2_mp (SEQ ID NO: 40), or a functional variant thereof;
CRE0009_S100B (SEQ ID NO: 23), or a functional variant thereof, and CRE0012_Arc (SEQ ID NO: 21), or a functional variant thereof, operably linked to CRE0004_GFAP_mp (SEQ ID NO: 41), or a functional variant thereof;
CRE0006_AQP4 (SEQ ID NO: 37), or a functional variant thereof, operably linked to CRE0003_CEND1_mp (SEQ ID NO: 25), or a functional variant thereof;
CRE0006_AQP4 (SEQ ID NO: 37), or a functional variant thereof, and CRE0012_Arc (SEQ ID NO: 21), or a functional variant thereof, operably linked to CRE0009_SYN1_mp (SEQ ID NO: 26), or a functional variant thereof;
CRE0006_AQP4 (SEQ ID NO: 37), or a functional variant thereof, and CRE0012_Arc (SEQ ID NO: 21), or a functional variant thereof, operably linked to CRE0015_GAP43 mp (SEQ ID NO: 39), or a functional variant thereof;
CRE0006_AQP4 (SEQ ID NO: 37), or a functional variant thereof, and CRE0012_Arc (SEQ ID NO: 21), or a functional variant thereof, operably linked to CRE0016_Eno2_mp (SEQ ID NO: 40), or a functional variant thereof;
CRE0006_AQP4 (SEQ ID NO: 37), or a functional variant thereof, and CRE0012_Arc (SEQ ID NO: 21), or a functional variant thereof, operably linked to CRE0004_GFAP_mp (SEQ ID NO: 41), or a functional variant thereof;
CRE0008_AQP4 (SEQ ID NO: 38), or a functional variant thereof, and CRE0012_Arc (SEQ ID NO: 21), or a functional variant thereof, operably linked to CRE0003_CEND1_mp (SEQ ID NO: 25), or a functional variant thereof;
CRE0008_AQP4 (SEQ ID NO: 38), or a functional variant thereof, and CRE0012_Arc (SEQ ID NO: 21), or a functional variant thereof, operably linked to CRE0009_SYN1_mp (SEQ ID NO: 26), or a functional variant thereof;
CRE0008_AQP4 (SEQ ID NO: 38), or a functional variant thereof, and CRE0012_Arc (SEQ ID NO: 21), or a functional variant thereof, operably linked to CRE0015_GAP43_mp (SEQ ID NO: 39), or a functional variant thereof;
CRE0008_AQP4 (SEQ ID NO: 38), or a functional variant thereof, and CRE0012_Arc (SEQ ID NO: 21), or a functional variant thereof, operably linked to CRE0016_Eno2_mp (SEQ ID NO: 40), or a functional variant thereof; or
CRE0008_AQP4 (SEQ ID NO: 38), or a functional variant thereof, and CRE0012_Arc (SEQ ID NO: 21), or a functional variant thereof, operably linked to CRE0004_GFAP_mp (SEQ ID NO: 41), or a functional variant thereof.

Suitably, the synthetic NS-specific promoter may comprise CRE0002_S100B (SEQ ID NO: 24), or a functional variant thereof. CRE0002_S100B (SEQ ID NO: 24) comprises CRE0009_S100B (SEQ ID NO: 23) and additional sequence, i.e. CRE0009_S100B (SEQ ID NO: 23) is a shorter version of CRE0002_S100B (SEQ ID NO: 24). Thus, CRE0002_S100B (SEQ ID NO: 24) can be viewed as a (longer) functional variant of CRE0009_S100B (SEQ ID NO: 23). In aspects or embodiments disclosed herein, CRE0002_S100B (SEQ ID NO: 24) can be provided in place of CRE0009_S100B (SEQ ID NO: 23).

Suitably, the synthetic NS-specific promoter may comprise CRE0005_GFAP (SEQ ID NO: 19), or a functional variant thereof. CRE0005_GFAP (SEQ ID NO: 19) comprises CRE0007_GFAP (SEQ ID NO: 20) and additional sequence, i.e. CRE0007_GFAP (SEQ ID NO: 20) is a shorter version of CRE0005_GFAP (SEQ ID NO: 19). Thus, CRE0005_GFAP (SEQ ID NO: 19) can be viewed as a (longer) functional variant of CRE0007_GFAP (SEQ ID NO: 20). In aspects or embodiments disclosed herein, CRE0005_GFAP (SEQ ID NO: 19) can be provided in place of CRE0007_GFAP (SEQ ID NO: 20).

Suitably, the synthetic NS-specific promoter may comprise CRE0006_GFAP (SEQ ID NO: 27). CRE0006_GFAP (SEQ ID NO: 27) comprises CRE0007_GFAP (SEQ ID NO: 20) and additional sequence, i.e. CRE0007_GFAP (SEQ ID NO: 20) is a shorter version of CRE0006_GFAP (SEQ ID NO: 27). Thus CRE0006_GFAP (SEQ ID NO: 27) can be viewed as a (longer) functional variant of CRE0007_GFAP (SEQ ID NO: 20). In aspects or embodiments disclosed herein, CRE0006_GFAP (SEQ ID NO: 27) can be provided in place of CRE0007_GFAP (SEQ ID NO: 20).

Suitably, the synthetic NS-specific promoter may comprise CRE0008_GFAP (SEQ ID NO: 28). CRE0008_GFAP (SEQ ID NO: 28) comprises CRE0007_GFAP (SEQ ID NO: 20) and additional sequence, i.e. CRE0007_GFAP (SEQ ID NO: 20) is a shorter version of CRE0008_GFAP (SEQ ID NO: 28). Thus CRE0008_GFAP (SEQ ID NO: 28) can be viewed as a (longer) functional variant of CRE0007_GFAP (SEQ ID NO: 20). In aspects or embodiments disclosed herein, CRE0008_GFAP (SEQ ID NO: 28) can be provided in place of CRE0007_GFAP (SEQ ID NO: 20).

Suitably, the synthetic NS-specific promoter comprises or consists of:
CRE0005_GFAP (SEQ ID NO: 19), or a functional variant thereof, operably linked to CRE0003_CEND1_mp (SEQ ID NO: 25), or a functional variant thereof;
CRE0005_GFAP (SEQ ID NO: 19), or a functional variant thereof, operably linked to CRE0009_SYN1_mp (SEQ ID NO: 26), or a functional variant thereof;
CRE0002_S100B (SEQ ID NO: 24), or a functional variant thereof, operably linked to CRE0003_CEND1_mp (SEQ ID NO: 25), or a functional variant thereof; or
CRE0002_S100B (SEQ ID NO: 24), or a functional variant thereof, operably linked to CRE0009_SYN1_mp (SEQ ID NO: 26), or a functional variant thereof.
CRE0005_GFAP (SEQ ID NO: 19), or a functional variant thereof, and CRE0012_Arc (SEQ ID NO: 21), or a functional variant thereof, operably linked to CRE0003_CEND1_mp (SEQ ID NO: 25), or a functional variant thereof;
CRE0005_GFAP (SEQ ID NO: 19), or a functional variant thereof, and CRE0012_Arc (SEQ ID NO: 21), or a functional variant thereof, operably linked to CRE0009_SYN1 mp (SEQ ID NO: 26), or a functional variant thereof;
CRE0002_S100B (SEQ ID NO: 24), or a functional variant thereof, and CRE0012_Arc (SEQ ID NO: 21), or a functional variant thereof, operably linked to CRE0003_CEND1_mp (SEQ ID NO: 25), or a functional variant thereof; or
CRE0002_S100B (SEQ ID NO: 24), or a functional variant thereof, and CRE0012_Arc (SEQ ID NO: 21), or a functional variant thereof, operably linked to CRE0009_SYN1_mp (SEQ ID NO: 26), or a functional variant thereof.

In one embodiment, the NS-specific promoter comprises CRE0003_CEND1_mp (SEQ ID NO: 25) and CRE0005_GFAP (SEQ ID NO: 19).

In one embodiment, the NS-specific promoter comprises CRE0009_SYN1_mp (SEQ ID NO: 26) and CRE0005_GFAP (SEQ ID NO: 19).

In one embodiment, the NS-specific promoter comprises CRE0003_CEND1_mp (SEQ ID NO: 25), CRE0012_Arc (SEQ ID NO: 21) and CRE0005_GFAP (SEQ ID NO: 19).

In one embodiment, the NS-specific promoter comprises CRE0009_SYN1_mp (SEQ ID NO: 26), CRE0012_Arc (SEQ ID NO: 21) and CRE0005_GFAP (SEQ ID NO: 19).

In one embodiment, the NS-specific promoter comprises CRE0003_CEND1_mp (SEQ ID NO: 25) and CRE0007_GFAP (SEQ ID NO: 20).

In one embodiment, the NS-specific promoter comprises CRE0009_SYN1_mp (SEQ ID NO: 26) and CRE0007_GFAP (SEQ ID NO: 20).

In one embodiment, the NS-specific promoter comprises CRE0003_CEND1_mp (SEQ ID NO: 25) and CRE0009_S100B (SEQ ID NO: 23).

In one embodiment, the NS-specific promoter comprises CRE0009_SYN1_mp (SEQ ID NO: 26) and CRE0009_S100B (SEQ ID NO: 23).

In one embodiment, the NS-specific promoter comprises CRE0003_CEND1_mp (SEQ ID NO: 25) and CRE0002_S100B (SEQ ID NO: 24).

In one embodiment, the NS-specific promoter comprises CRE0003_CEND1_mp (SEQ ID NO: 25) and CRE0001_S100B (SEQ ID NO: 22).

In one embodiment, the NS-specific promoter comprises CRE0009_SYN1_mp (SEQ ID NO: 26) and CRE0002_S100B (SEQ ID NO: 24).

In one embodiment, the NS-specific promoter comprises CRE0009_SYN1_mp (SEQ ID NO: 26) and CRE0001_S100B (SEQ ID NO: 22).

In one embodiment, the NS-specific promoter comprises CRE0003_CEND1_mp (SEQ ID NO: 25), CRE0002_S100B (SEQ ID NO: 24) and CRE0012_Arc (SEQ ID NO: 21).

In one embodiment, the NS-specific promoter comprises CRE0009_SYN1_mp (SEQ ID NO: 26), CRE0002_S100B (SEQ ID NO: 24) and CRE0012_Arc (SEQ ID NO: 21).

In one embodiment, the NS-specific promoter comprises CRE0006_AQP4 (SEQ ID NO: 37) and CRE0004_GFAP_mp (SEQ ID NO: 41).

In one embodiment, the NS-specific promoter comprises CRE0008_AQP4 (SEQ ID NO: 38) and CRE0015_GAP43_mp (SEQ ID NO: 39).

In one embodiment, NS-specific promoter comprises CRE0006_AQP4 (SEQ ID NO: 37) and CRE0015_GAP43_mp (SEQ ID NO: 39).

In one embodiment, NS-specific promoter comprises CRE0006_AQP4 (SEQ ID NO: 37) and CRE0016_Eno2_mp (SEQ ID NO: 40).

It is generally preferred that a promoter according to the present invention which comprises a variant CRE of any one of SEQ ID NOs 19-24, 27, 28, 37, 38 retains at least 25%, 50%, 75%, 80%, 85%, 90%, 95% or 100% of the activity of the reference CRE. Retention of activity can be assessed by comparing expression of a suitable reporter under the control of the reference promoter with an otherwise identical promoter comprising the substituted CRE under equivalent conditions. Suitably said activity is assessed using the examples as described herein, but other methods can be used.

It is generally preferred that a promoter according to the present invention which comprises a variant promoter element of any one of SEQ ID NOs 25-26, 29-32, 39-41 retains at least 25%, 50%, 75%, 80%, 85%, 90%, 95% or 100% of the activity of the reference promoter element. Suitably said activity is assessed using the examples as described herein, but other 30 methods can be used.

Suitably the CRE is a nervous system specific CRE, suitably a CNS-specific cis-regulatory element and/or a PNS-specific cis-regulatory element.

Suitably the promoter element is a minimal or proximal promoter. Preferably, when present, the proximal promoter is a NS-specific promoter element, suitably a CNS-specific proximal promoter and/or a PNS-specific proximal promoter.

In some embodiments, the synthetic NS-specific promoter comprises or consists of a sequence according to any one of SEQ ID NOs 1-14, 33-36 or a functional variant thereof.

In some embodiments the synthetic NS-specific promoter comprises or consists of a sequence which is at least 60%, 65%, 70%, 75%, 80%, 85%, 90%, 95%, 96%, 97%, 98% or 99% identical to any one of SEQ ID NOs 1-14, 33-36.

The present invention thus provides various synthetic NS-specific promoters and functional variants thereof. It is generally preferred that a promoter according to the present invention which is a variant of any one of SEQ ID NO 1-14, 33-36 retains at least 25%, 50%, 75%, 80%, 85%, 90%, 95% or 100% of the activity of the reference promoter. Suitably said activity is assessed using the examples as described herein, but other methods can be used.

In one embodiment, the synthetic NS-specific promoter comprises or consists of SEQ ID NO: 1, or a functional variant thereof, and is active in neurones and astrocytes.

In one embodiment, the synthetic NS-specific promoter comprises or consists of SEQ ID NO: 2, or a functional variant thereof, and is active in neurones and astrocytes.

In one embodiment, the synthetic NS-specific promoter comprises or consists of SEQ ID NO: 3, or a functional variant thereof, and is active in excitatory neurones and astrocytes.

In one embodiment, the synthetic NS-specific promoter comprises or consists of SEQ ID NO: 4, or a functional variant thereof, and is active in excitatory neurones and astrocytes.

In one embodiment, the synthetic NS-specific promoter comprises or consists of SEQ ID NO: 5, or a functional variant thereof, and is active in neurones and astrocytes.

In one embodiment, the synthetic NS-specific promoter comprises or consists of SEQ ID NO: 6, or a functional variant thereof, and is active in neurones and astrocytes.

In one embodiment, the synthetic NS-specific promoter comprises or consists of SEQ ID NO: 7, or a functional variant thereof, and is active in neurones, astrocytes and oligodendrocytes.

In one embodiment, the synthetic NS-specific promoter comprises or consists of SEQ ID NO: 8, or a functional variant thereof, and is active in neurones, astrocytes and oligodendrocytes.

In one embodiment, the synthetic NS-specific promoter comprises or consists of SEQ ID NO: 9, or a functional variant thereof, and is active in neurones, astrocytes and oligodendrocytes.

In one embodiment, the synthetic NS-specific promoter comprises or consists of SEQ ID NO: 10, or a functional variant thereof, and is active in neurones, astrocytes and oligodendrocytes.

In one embodiment, the synthetic NS-specific promoter comprises or consists of SEQ ID NO: 11, or a functional variant thereof, and is active in neurones, astrocytes and oligodendrocytes.

In one embodiment, the synthetic NS-specific promoter comprises or consists of SEQ ID NO: 12, or a functional variant thereof, and is active in neurones, astrocytes and oligodendrocytes.

In one embodiment, the synthetic NS-specific promoter comprises or consists of SEQ ID NO: 13, or a functional variant thereof, and is active in excitatory neurones, astrocytes and oligodendrocytes.

In one embodiment, the synthetic NS-specific promoter comprises or consists of SEQ ID NO: 14, or a functional variant thereof, and is active in excitatory neurones, astrocytes and oligodendrocytes.

In one embodiment, the synthetic NS-specific promoter comprises or consists of SEQ ID NO: 33, or a functional variant thereof, and is active in neurones and astrocytes.

In one embodiment, the synthetic NS-specific promoter comprises or consists of SEQ ID NO: 34, or a functional variant thereof, and is active in neurones and astrocytes.

In one embodiment, the synthetic NS-specific promoter comprises or consists of SEQ ID NO: 35, or a functional variant thereof, and is active in neurones and astrocytes.

In one embodiment, the synthetic NS-specific promoter comprises or consists of SEQ ID NO: 36, or a functional variant thereof, and is active in neurones and astrocytes.

In some embodiments, synthetic NS-specific promoters SP0013 (SEQ ID NO: 1), SP0014 (SEQ ID NO: 2), SP0030 (SEQ ID NO: 5), SP0031 (SEQ ID NO: 6), SP0032 (SEQ ID NO: 7), SP0019 (SEQ ID NO: 9), SP0020 (SEQ ID NO: 10), SP0021 (SEQ ID NO: 11), SP0022 (SEQ ID NO: 12), SP0011 (SEQ ID NO: 33), SP0034 (SEQ ID NO: 34), SP0035 (SEQ ID NO: 35) and SP0036 (SEQ ID NO: 36) are particularly preferred. In some embodiments, the synthetic NS-specific promoter comprises or consists of SEQ ID NO: 1, SEQ ID NO: 2, SEQ ID NO: 5, SEQ ID NO: 6, SEQ ID NO: 7, SEQ ID NO: 9, SEQ ID NO: 10, SEQ ID NO: 11, SEQ ID NO: 12, SEQ ID NO: 33, SEQ ID NO: 34, SEQ ID NO: 35 or SEQ ID NO: 36.

In one embodiment, the synthetic NS-specific promoter comprises a CNS-specific CRE which is active in astrocytes, suitably the CRE may be selected from: CRE0001_S100B (SEQ ID NO: 22), CRE0009_S100B (SEQ ID NO: 23), CRE0007_GFAP (SEQ ID NO: 20), CRE0006_GFAP (SEQ ID NO: 27), CRE0008_GFAP (SEQ ID NO: 28), CRE0002_S100B (SEQ ID NO: 24), CRE0006_AQP4 (SEQ ID NO: 37), CRE0008_AQP4 (SEQ ID NO: 38) and CRE0005_GFAP (SEQ ID NO: 19).

In one embodiment, the synthetic NS-specific promoter comprises a CNS-specific CRE which is active in oligodendrocytes, suitably the CRE may be selected from CRE0001_S100B (SEQ ID NO: 22), CRE0002_S100B (SEQ ID NO: 24) or CRE0009_S100B (SEQ ID NO: 23).

In one embodiment, the synthetic NS-specific promoter comprises a CRE which is active in excitatory neurones, suitably the CRE may be CRE0012_Arc (SEQ ID NO: 21).

In one embodiment, the synthetic NS-specific promoter comprises a promoter element which is active in neurones, suitably the promoter element may be selected from CRE0003_CEND1_mp (SEQ ID NO: 25), CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0015_GAP43_mp (SEQ ID NO: 39), CRE0016_Eno2 mp (SEQ ID NO: 40) or CRE0013_SYN1 mp (SEQ ID NO: 32).

In one embodiment, the synthetic NS-specific promoter comprises a promoter element which is active in astrocytes, suitably the promoter element may be CRE0004_GFAP_mp (SEQ ID NO: 41).

In another aspect of the present invention, there is provided a NS-specific cis-regulatory element (CRE) comprising or consisting of a sequence according to any one of SEQ ID NOs: 19-24, 27, 28, 37, 38 or a functional variant of any thereof. In some embodiments the NS-specific CRE comprises a sequence which is at least 60%, 65%, 70%, 75%, 80%, 85%, 90%, 95%, 96%, 97%, 98% or 99% identical to any one of SEQ ID NOs 19-24, 27, 28, 37, 38.

The NS-specific CRE may be active in the CNS when operably linked to a promoter element, i.e. CNS-specific CRE. The NS-specific CRE may be active in the PNS when operably linked to a promoter element, i.e. PNS-specific CRE. The NS-specific CRE may be active in the CNS and PNS when operably linked to a promoter element.

It is generally preferred that a NS-specific CRE according to the present invention which is a variant of any one of SEQ ID NOs 19-24, 27, 28, 37, 38 retains at least 25%, 50%, 75%, 80%, 85%, 90%, 95% or 100% of the activity of the reference CRE. Retention of activity can be assessed by comparing expression of a suitable reporter under the control of the reference promoter with an otherwise identical promoter comprising the substituted CRE under equivalent conditions. Suitably said activity is assessed using the examples as described herein, but other methods can be used.

Suitably, the CRE according to the present invention or a functional variant thereof may be as short as possible while still maintaining activity. Suitably the CRE according to the present invention or a functional variant thereof may be defined as ‘shortened’ and may have a length of less than 600 bp, less than 550 bp, less than 500 bp, less than 450 bp, or less than 400 bp. Preferably, the CRE according to the present invention or a functional variant thereof has a length of 500 or fewer nucleotides, more preferably 450 or fewer nucleotides.

Suitably, the CRE according to the present invention may be operably linked to a promoter element. The promoter element may be a minimal or a proximal promoter. When present, preferably, the proximal promoter is a NS-specific proximal promoter, suitably a CNS-specific proximal promoter. Suitably, the promoter element may be CRE0003_CEND1_mp (SEQ ID NO: 25), CRE0009_SYN1 mp (SEQ ID NO: 26), CRE0001_CEND1 mp (SEQ ID NO: 29), CRE0002_CEND1_mp (SEQ ID NO: 30), CRE0008_SYN1_mp (SEQ ID NO: 31), CRE0015_GAP43_mp (SEQ ID NO: 39), CRE0016_Eno2_mp (SEQ ID NO: 40), CRE0004_GFAP_mp (SEQ ID NO: 41) or CRE0013_SYN1_mp (SEQ ID NO: 32), or a functional variant thereof.

The CNS-specific cis-regulatory element (CRE) may be active in astrocytes when operably linked to a minimal or proximal promoter, suitably the CRE may be selected from: CRE0001_S100B (SEQ ID NO: 22), CRE0009_S100B (SEQ ID NO: 23), CRE0007_GFAP (SEQ ID NO: 20), CRE0006_GFAP (SEQ ID NO: 27), CRE0008_GFAP (SEQ ID NO: 28), CRE0002_S100B (SEQ ID NO: 24), CRE0006_AQP4 (SEQ ID NO: 37), CRE0008_AQP4 (SEQ ID NO: 38) and CRE0005_GFAP (SEQ ID NO: 19). In some embodiments, the CRE which is active in astrocytes when operably linked to a minimal or proximal promoter, or a functional variant thereof, may have a length of less than 600 bp, less than 550 bp, less than 500 bp, less than 450 bp, or less than 400 bp. The CRE which is active in astrocytes when operably linked to a minimal or proximal promoter, or a functional variant thereof, preferably has a length of 500 or fewer nucleotide, more preferably 400 or fewer nucleotides. The CNS-specific CRE according to the present invention may be active in astrocytes and shortened, i.e. selected from: CRE0006_GFAP (SEQ ID NO: 27), CRE0008_GFAP (SEQ ID NO: 28), CRE0007_GFAP (SEQ ID NO: 20), CRE0001_S100B (SEQ ID NO: 22), CRE0009_S100B (SEQ ID NO: 23) and CRE0002_S100B (SEQ ID NO: 24).

The CNS-specific cis-regulatory element (CRE) may be active in oligodendrocytes when operably linked to a minimal or proximal promoter, suitably the CRE may be selected from CRE0001_S100B (SEQ ID NO: 22), CRE0002_S100B (SEQ ID NO: 24) or CRE0009_S100B (SEQ ID NO: 23). In some embodiments, the CRE which is active in oligodendrocytes when operably linked to a minimal or proximal promoter, or a functional variant thereof, may have length of less than 600 bp, less than 550 bp, less than 500 bp, less than 450 bp, or less than 400 bp. The CRE which is active in oligodendrocytes when operably linked to a minimal or proximal promoter, or a functional variant thereof, is preferably 500 or fewer nucleotide, more preferably 400 or fewer nucleotides. The CNS-specific CRE according to the present invention may be active in oligodendrocytes and shortened, i.e. selected from: CRE0001_S100B (SEQ ID NO: 22), CRE0009_S100B (SEQ ID NO: 23) and CRE0002_S100B (SEQ ID NO: 24).

The NS-specific cis-regulatory element (CRE) may be active in excitatory neurones when operably linked to a minimal or proximal promoter, suitably the CRE may be CRE0012_Arc (SEQ ID NO: 21).

The NS-specific CRE according to the present invention is preferably shortened, i.e. selected from: CRE0006_GFAP (SEQ ID NO: 27), CRE0008_GFAP (SEQ ID NO: 28), CRE0007_GFAP (SEQ ID NO: 20), CRE0001_S100B (SEQ ID NO: 22), CRE0009_S100B (SEQ ID NO: 23) and CRE0002_S100B (SEQ ID NO: 24).

Suitably, the CRE according to the present invention may be combined with one or more additional CREs to form a cis-regulatory module (CRM). Suitably, the one or more additional CREs may be CREs according to SEQ ID NOs 19-24, 27, 28, or functional variants thereof, or they can be other CREs. Suitably, when the one or more additional CREs are not CREs according to the present invention, the one or more additional CRE may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. Suitably, the additional CREs are NS-specific, suitably CNS-specific and/or PNS-specific.

In another aspect of the present invention there is provided a synthetic NS-specific promoter comprising or consisting of a CRE according to any one of SEQ ID NOs 19-24, 27, 28, 37, 38 or a functional variant thereof. In some embodiments, the CRE may be operably linked to a promoter element. In some embodiments, the promoter element may be a minimal or a proximal promoter. Preferably, the proximal promoter is a NS-specific proximal promoter, suitably a CNS-specific and/or PNS-specific proximal promoter. Suitably, the promoter element may be CRE0003_CEND1_mp (SEQ ID NO: 25), CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0015_GAP43_mp (SEQ ID NO: 39), CRE0016_Eno2_mp (SEQ ID NO: 40), CRE0004_GFAP_mp (SEQ ID NO: 41) or CRE0013_SYN1_mp (SEQ ID NO: 32), or a functional variant thereof.

In a further aspect of the present invention, there is provided a NS-specific cis-regulatory module (CRM) comprising a CRE according to the present invention. Suitably, the CRM comprises or consists of any one of SEQ ID NO: 15-18, or a functional variant thereof.

The NS-specific CRM may be active in the CNS when operably linked to a promoter element, i.e. CNS-specific CRM. The NS-specific CRM may be active in the PNS when operably linked to a promoter element, i.e. PNS-specific CRM. The NS-specific CRM may be active in the CNS and PNS when operably linked to a promoter element.

Suitably, the synthetic NS-specific CRM comprises or consists of a sequence which is at least 70%, 75%, 80%, 85%, 90%, 95%, 96%, 97%, 98% or 99% identical to any one of SEQ ID NOs: 15-18.

The CRM according to the present invention may be operably linked to a promoter element. The promoter element may be a minimal or a proximal promoter. When present, preferably, the proximal promoter is a NS-specific proximal promoter, suitably a CNS-specific and/or PNS-specific proximal promoter. Suitably, the promoter element may be CRE0003_CEND1_mp (SEQ ID NO: 25), CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0015_GAP43_mp (SEQ ID NO: 39), CRE0016_Eno2_mp (SEQ ID NO: 40), CRE0004_GFAP_mp (SEQ ID NO: 41) or CRE0013_SYN1_mp (SEQ ID NO: 32), or a functional variant thereof.

A synthetic NS-specific promoter according to the present invention may comprise a CRM according to the present invention operably linked to a promoter element (typically a minimal or proximal promoter). The proximal promoter is preferably a NS-specific proximal promoter, suitably a CNS-specific and/or PNS-specific promoter element. Suitably, the promoter element may be CRE0003_CEND1_mp (SEQ ID NO: 25), CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0015_GAP43_mp (SEQ ID NO: 39), CRE0016_Eno2_mp (SEQ ID NO: 40), CRE0004_GFAP_mp (SEQ ID NO: 41) or CRE0013_SYN1_mp (SEQ ID NO: 32), or a functional variant thereof.

Suitably, the CRM comprises at least one of the following cis-regulatory elements (CREs) or functional variants thereof:
CRE0001_S100B (SEQ ID NO: 22) or a functional variant thereof;
CRE0002_S100B (SEQ ID NO: 24) or a functional variant thereof;
CRE0005_GFAP (SEQ ID NO: 19) or a functional variant thereof;
CRE0007_GFAP (SEQ ID NO: 20) or a functional variant thereof;
CRE0009_S100B (SEQ ID NO: 23) or a functional variant thereof;
CRE0012_Arc (SEQ ID NO: 21) or a functional variant thereof;
CRE0006_GFAP (SEQ ID NO: 27) or functional variant thereof;
CRE0006_AQP4 (SEQ ID NO: 37) or functional variant thereof;
CRE0008_AQP4 (SEQ ID NO: 38) or functional variant thereof; and
CRE0008_GFAP (SEQ ID NO: 28) or functional variant thereof.

Suitably, the CRM comprises at least one of the following cis-regulatory elements (CREs) or functional variants thereof:
CRE0001_S100B (SEQ ID NO: 22), or a functional variant thereof;
CRE0007_GFAP (SEQ ID NO: 20), or a functional variant thereof;
CRE0006_AQP4 (SEQ ID NO: 37) or functional variant thereof;
CRE0008_AQP4 (SEQ ID NO: 38) or functional variant thereof; and
CRE0009_S100B (SEQ ID NO: 23), or a functional variant thereof.

Suitably, the CRM may comprise CRE0002_S100B (SEQ ID NO: 24), or a functional variant thereof, CRE0005_GFAP (SEQ ID NO: 19), or a functional variant thereof, CRE0006_GFAP (SEQ ID NO: 27) or a functional variant thereof, CRE0008_GFAP (SEQ ID NO: 28) or a functional variant thereof, CRE0006_AQP4 (SEQ ID NO: 37) or functional variant thereof, CRE0008_AQP4 (SEQ ID NO: 38) or functional variant thereof.

Suitably, the CRM may comprise one or more additional CREs according to the present invention or other NS-specific CREs. Suitably, when the one or more additional CREs are not CREs according to the present invention, the one or more additional CREs may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. Suitably, the CRM may additionally comprise CRE0012_Arc (SEQ ID NO: 21), or a functional variant thereof.

Suitably, the synthetic NS-specific CRM comprises or consists of:
CRE0001_S100B (SEQ ID NO: 22), or a functional variant thereof, and CRE0012_Arc (SEQ ID NO: 21), or a functional variant thereof;
CRE0007_GFAP (SEQ ID NO: 20), or a functional variant thereof, and CRE0012_Arc (SEQ ID NO: 21), or a functional variant thereof;
CRE0006_AQP4 (SEQ ID NO: 37), or a functional variant thereof, and CRE0012_Arc (SEQ ID NO: 21), or a functional variant thereof;
CRE0008_AQP4 (SEQ ID NO: 38), or a functional variant thereof, and CRE0012_Arc (SEQ ID NO: 21), or a functional variant thereof; or
CRE0009_S100B (SEQ ID NO: 23), or a functional variant thereof, and CRE0012_Arc (SEQ ID NO: 21), or a functional variant thereof.

In one embodiment, the NS-specific CRM comprises CRE0012_Arc (SEQ ID NO: 21) and CRE0005_GFAP (SEQ ID NO: 19). Suitably, in such an embodiment, the CRM may comprise or consist of SEQ ID NO: 15 or SEQ ID NO: 16. Suitably, the NS-specific CRM is active in excitatory neurones and astrocytes when operably linked to a minimal promoter.

In one embodiment, the NS-specific CRM comprises CRE0002_S100B (SEQ ID NO: 24) and CRE0012_Arc (SEQ ID NO: 21). Suitably, in such an embodiment, the CRM may comprise or consist of SEQ ID NO: 17 or SEQ ID NO: 18. Suitably, the NS-specific CRM is active in excitatory neurones, oligodendrocytes and astrocytes when operably linked to a minimal promoter.

In one embodiment, the synthetic NS-specific CRM comprises a CRE which is active in astrocytes, suitably the CRE may be selected from: CRE0001_S100B (SEQ ID NO: 22), CRE0009_S100B (SEQ ID NO: 23), CRE0007_GFAP (SEQ ID NO: 20), CRE0002_S100B (SEQ ID NO: 24), CRE0006_AQP4 (SEQ ID NO: 37), CRE0008_AQP4 (SEQ ID NO: 38) and CRE0005_GFAP (SEQ ID NO: 19).

In one embodiment, the synthetic NS-specific CRM comprises a CRE which is active in oligodendrocytes, suitably the CRE may be selected from CRE0001_S100B (SEQ ID NO: 22), CRE0002_S100B (SEQ ID NO: 24) or CRE0009_S100B (SEQ ID NO: 23).

In one embodiment, the synthetic NS-specific CRM comprises a CRE which is active in excitatory neurones, suitably the CRE may be CRE0012_Arc (SEQ ID NO: 21).

In a further aspect of the present invention, there is provided a promoter element (a minimal or a proximal promoter) comprising or consisting of a sequence according to any one of SEQ ID NOs: 25-26, 29-32, 39-41 or a functional variant thereof.

The promoter element may be active in the CNS, i.e. CNS-specific promoter element. The promoter element may be active in the PNS, i.e. PNS-specific promoter element. The promoter element may be active in the PNS and CNS.

In another aspect of the present invention, there is provided a synthetic promoter comprising said minimal or proximal promoter, suitably a synthetic NS-specific promoter comprising said minimal or proximal promoter. Suitably a functional variant of the minimal or proximal promoter comprises a sequence which is at least 60%, 65%, 70%, 75%, 80%, 85%, 90%, 95%, 96%, 97%, 98%, 99% identical to SEQ ID NOs: 25-26, 29-32, 39-41.

Suitably, the promoter element according to the present invention or a functional variant thereof may be as short as possible while still maintaining activity. Suitably the promoter element according to the present invention or a functional variant thereof may be defined as ‘shortened’ and may have length of less than 500 bp, less than 450 bp, less than 400 bp, less than 350 bp, less than 300 bp, less than 250 bp, less than 200 bp, less than 150 bp, or less than 100 bp. Preferably the promoter element according to the present invention or functional variant thereof has a length of 350 or fewer nucleotides, more preferably 300 or fewer nucleotides.

The minimal or proximal promoter may be active in neurones, suitably the promoter element may be selected from CRE0003_CEND1_mp (SEQ ID NO: 25) or CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0015_GAP43_mp (SEQ ID NO: 39), CRE0016_Eno2_mp (SEQ ID NO: 40), or CRE0013_SYN1_mp (SEQ ID NO: 32), or a functional variant thereof. Suitably, the neurones may be CNS neurones and/or PNS neurones. Suitably, the minimal or proximal promoter which is active in neurones or a functional variant thereof is less than 500 bp, less than 450 bp, less than 400 bp, less than 350 bp, less than 300 bp, less than 250 bp, less than 200 bp, less than 150 bp, or less than 100 bp in length. Preferably, the minimal or proximal promoter which is active in neurones or a functional variant thereof has a length of 300 or fewer nucleotides, more preferably 250 or fewer nucleotides.

The minimal or proximal promoter may be active in astrocytes, suitably the promoter element may be CRE0004_GFAP_mp (SEQ ID NO: 41), or a functional variant thereof. Suitably, the minimal or proximal promoter which is active in astrocytes or a functional variant thereof is less than 500 bp, less than 450 bp, less than 400 bp, less than 350 bp, less than 300 bp, less than 250 bp, less than 200 bp, less than 150 bp, or less than 100 bp in length.

Preferably, the minimal or proximal promoter which is active in astrocytes or a functional variant thereof has a length of 300 or fewer nucleotides, more preferably 250 or fewer nucleotides.

The promoter element according to the present invention is preferably shortened, i.e. selected from: CRE0003_CEND1_mp (SEQ ID NO: 25), CRE0009_SYN1_mp (SEQ ID NO: 26) and CRE0013_SYN1_mp (SEQ ID NO: 32).

The promoter element according to the present invention may consist or comprise of CRE0001_CEND1 mp (SEQ ID NO: 29), CRE0002_CEND1_mp (SEQ ID NO: 30) or CRE0003_CEND1_mp (SEQ ID NO: 25). CRE0003_CEND1_mp (SEQ ID NO: 25) comprises CRE0001_CEND1_mp (SEQ ID NO: 29) and additional sequence, i.e. CRE0001_CEND1_mp (SEQ ID NO: 29) is a shorter version of CRE0003_CEND1_mp (SEQ ID NO: 25). CRE0002_CEND1_mp (SEQ ID NO: 30) comprises CRE0001_CEND1_mp (SEQ ID NO: 29) and additional sequence, i.e. CRE0001_CEND1_mp (SEQ ID NO: 29) is a shorter version of CRE0002_CEND1_mp (SEQ ID NO: 30). Similarly, CRE0002_CEND1_mp (SEQ ID NO: 30) comprises CRE0003_CEND1_mp (SEQ ID NO: 25) and additional sequence, i.e. CRE0003_CEND1_mp (SEQ ID NO: 25) is a shorter version of CRE0002_CEND1_mp (SEQ ID NO: 30). In aspects or embodiments disclosed herein, CRE0001_CEND1_mp (SEQ ID NO: 29), CRE0002_CEND1_mp (SEQ ID NO: 30) or CRE0003_CEND1_mp (SEQ ID NO: 25) can be provided in place of each other.

The promoter element according to the present invention may consist or comprise of CRE0008_SYN1_mp (SEQ ID NO: 31), CRE0013_SYN1_mp (SEQ ID NO: 32) or CRE0009_SYN1_mp (SEQ ID NO: 26). CRE0009_SYN1_mp (SEQ ID NO: 26) comprises CRE0013_SYN1_mp (SEQ ID NO: 32) and additional sequence, i.e. CRE0013_SYN1_mp (SEQ ID NO: 32) is a shorter version of CRE0009_SYN1_mp (SEQ ID NO: 26). CRE0008_SYN1_mp (SEQ ID NO: 31) comprises CRE0013_SYN1_mp (SEQ ID NO: 32) and additional sequence, i.e. CRE0013_SYN1_mp (SEQ ID NO: 32) is a shorter version of CRE0008_SYN1_mp (SEQ ID NO: 31). Similarly, CRE0008_SYN1_mp (SEQ ID NO: 31) comprises CRE0009_SYN1_mp (SEQ ID NO: 26) and additional sequence, i.e. CRE0009_SYN1_mp (SEQ ID NO: 26) is a shorter version of CRE0008_SYN1_mp (SEQ ID NO: 31). In aspects or embodiments disclosed herein, CRE0008_SYN1_mp (SEQ ID NO: 31), CRE0013_SYN1_mp (SEQ ID NO: 32) or CRE0009_SYN1_mp (SEQ ID NO: 26) can be provided in place of each other.

Suitably the minimal or proximal promoter can be operably linked with a CRE or CRM. The CRE may be a CRE according to this invention or any other CRE. Suitably, when the CRE is not a CRE according to the present invention, the CRE may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. The CRM may be a CRM according to this invention or may comprise a CRE according to this invention. Suitably, the CRE or the CRM is NS-specific, suitably CNS-specific and/or PNS-specific.

Suitably the proximal promoter according to the present invention may be operably linked with one or more proximal promoters. A synthetic NS-specific promoter according to the present invention may comprise or consist of two proximal promoters. Suitably, a synthetic NS-specific promoter according to the present invention may comprise or consist of two or more proximal promoters. Suitably, the proximal promoters are NS-specific proximal promoters, suitably CNS-specific proximal promoters and/or PNS-specific proximal promoters. Suitably, the proximal promoter may be selected from CRE0003_CEND1_mp (SEQ ID NO: 25), CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0015_GAP43_mp (SEQ ID NO: 39), CRE0016_Eno2_mp (SEQ ID NO: 40), CRE0004_GFAP_mp (SEQ ID NO: 41) or CRE0013_SYN1_mp (SEQ ID NO: 32), or a functional variant thereof. Suitably a synthetic NS-specific promoter according to the present invention may comprise or consist of any two of the following group: CRE0003_CEND1_mp (SEQ ID NO: 25), CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0015_GAP43_mp (SEQ ID NO: 39), CRE0016_Eno2_mp (SEQ ID NO: 40), CRE0004_GFAP_mp (SEQ ID NO: 41) and CRE0013_SYN1_mp (SEQ ID NO: 32), or a functional variant thereof. Suitably, the at least two proximal promoters may be operably linked to a CRE or a CRM according to the present invention or other CREs or CRMs.

The CREs, minimal/proximal promoters or promoters of the present invention can be active in the CNS and/or the PNS. In some embodiments, the CREs, minimal/proximal promoters or synthetic promoters of the present invention are be active in the CNS, i.e. CNS-specific CREs, minimal/proximal promoters or synthetic promoters. In some embodiments, the CREs, minimal/proximal promoters or synthetic promoters of the present invention are active in the PNS, i.e. PNS-specific CREs, minimal/proximal promoters or synthetic promoters. In some embodiments, CREs, minimal/proximal promoters or promoters of the present invention are active in the CNS and the PNS.

The CREs, minimal/proximal promoters or promoters of the present invention can be active in specific region of the CNS, preferably in a specific region in the CNS, or in specific CNS cell type or in a combination of CNS cell types or in a combination of both. Suitably therefore the CREs, minimal/proximal promoters, or promoters of the present invention are CNS-specific.

The CREs, minimal/proximal promoters or promoters of the present invention can be active in specific region of the PNS, preferably in a specific region in the PNS, or in specific PNS cell type or in a combination of PNS cell types or in a combination of both. Suitably therefore the CREs, minimal/proximal promoters, or promoters of the present invention are PNS-specific.

The CREs, minimal/proximal promoters or promoters of the present invention can be active in one or more of the various parts of the NS, suitably one or more of the various parts of the CNS and/or one or more of the various parts of the PNS.

The CREs, minimal/proximal promoters or promoters of the present invention can be active in one or more of the various parts of the CNS. The CNS consists primarily of the brain and the spinal cord. The retina, optic nerve, olfactory nerves, and olfactory epithelium are sometimes considered to be part of the CNS alongside the brain and spinal cord. This is because they connect directly with brain tissue without intermediate nerve fibres. Suitably, the CREs, minimal/proximal promoter or promoters of the present invention may be active in the brain and the spinal cord. Suitably, the CREs, minimal/proximal promoter or promoters of the present invention may be active in the brain but not in the spinal cord or any other part of the CNS. Suitably, the CREs, minimal/proximal promoter or promoters of the present invention may be active in the spinal cord but not in the brain. Preferably the CREs, minimal/proximal promoter or promoters of the present invention may be active in the brain and the spinal cord. Suitably the CREs, minimal/proximal promoter or promoters of the present invention may be active in the brain. Suitably the CREs, minimal/proximal promoter or promoters of the present invention may be active in one or more of the various areas within the brain.

Non-limiting examples of brain areas include: frontal lobe, pariental lobe, occipital lobe, temporal lobe (which includes the hippocampus and amygdala), cerebellum, midbrain (which includes the basal ganglia, which in turn includes the striatum and substantia nigra), pons, medulla and the diencephalon (which includes the thalamus and hypothalamus). Non-limiting examples of spinal cord areas include: cervical vertebrae, thoracic vertebrae, lumbar vertebrae, sacrum vertebrae and coccyx vertebrae. In some embodiments, it may be desirable that the CRE, CRM, minimal/proximal promoter or promoter of the present invention shows widespread activity in the brain. In some embodiments, it may be desirable that the CRE, CRM, minimal/proximal promoter or promoter of the present invention shows widespread activity in the brain and the spinal cord. In some embodiments, the CRE, CRM, minimal/proximal promoter or promoter of the present invention is active in all parts of the brain or CNS (pan-CNS), preferably in all areas of the brain. In some embodiments the CRE, CRM, minimal/proximal promoter or promoter of the present invention is active in the brain but not in other parts of the CNS, e.g., the spinal cord. In some embodiments the CRE, CRM, minimal/proximal promoter or promoter of the present invention is active in the spinal cord but not in other parts of the CNS, e.g., the brain. In some embodiments, the CRE, CRM, minimal/proximal promoter or promoter of the present invention is active in 1, 2, 3, 4, or 5 of the areas of the spinal cord recited above. In some embodiments, the CRE, CRM, minimal/proximal promoter or promoter of the present invention is active in the majority of the areas in the spinal cord, i.e. at least 3, at least 4 or all 5 of the 5 areas of the brain recited above. In some embodiments, the CRE, CRM, minimal/proximal promoter or promoter of the present invention is active in 1, 2, 3, 4, 5, 6, 7, 8 or 9 of the areas of the brain recited above. In some embodiments, the CRE, CRM, minimal/proximal promoter or promoter of the present invention is active in 1, 2, 3, 4, 5, 6, 7, 8 or 9 of the areas of the brain recited above and in the spinal cord. In some embodiments, the CRE, CRM, minimal/proximal promoter or promoter of the present invention is active in the majority of the areas in the brain, i.e. at least 5, at least 6, at least 7, at least 8 or all 9 of the 9 areas of the brain recited above. In some embodiments, the CRE, CRM, minimal/proximal promoter or promoter of the present invention is active in the majority of the areas in the brain, i.e. at least 5, at least 6, at least 7, at least 8 or all 9 of the 9 areas of the brain recited above and in the spinal cord. In some embodiments, the CRE, CRM, minimal/proximal promoter or promoter of the present invention is active in from 4 to 6 areas of the brain recited above. In some embodiments, the CRE, CRM minimal/proximal promoter or promoter of the present invention is active in from 2 to 4 areas of the brain recited above. Suitably, the CRE, CRM, minimal/proximal promoter or promoter of the present invention is preferably active in any or all of the following brain regions: the frontal lobe, pariental lobe, occipital love, temporal lobe (which includes the hippocampus), cerebellum, midbrain (which includes the basal ganglia which in turn includes the striatum and the substantia nigra) and the diencephalon (which includes the thalamus and the hypothalamus). This may be particularly preferred as the striatum, the substantia nigra, layers 3, 5 and 6 of the cerebral cortex (the cerebral cortex includes the frontal lobe, pariental lobe, occipital lobe and temporal lobe), the hippocampus, the cerebellum, the hypothalamus and the thalamus, which are regions of the brain that are worst affected in Huntington's disease. Suitably, the CRE, CRM, minimal/proximal promoter or promoter of the present invention is preferably active in the spinal cord and in any or all of the following brain regions: the frontal lobe, pariental lobe, occipital love, temporal lobe (which includes the hippocampus), cerebellum, midbrain (which includes the basal ganglia which in turn includes the striatum and the substantia nigra) and the diencephalon (which includes the thalamus and the hypothalamus).

The CREs, minimal/proximal promoters or promoters of the present invention can be active in one or more of the various parts of the PNS. The PNS refers to the parts of the nervous system which are outside the brain and spinal cord. Non-limiting examples of peripheral nervous system include cranial nerves, brachial plexus, thoracoabdominal nerves, lumbar plexus, sacral plexus and neuromuscular junctions. In some embodiments, it may be desirable that the CRE, CRM, minimal/proximal promoter or promoter of the present invention shows widespread activity in the PNS. In some embodiments, the CRE, CRM, minimal/proximal promoter or promoter of the present invention is active in 1, 2, 3, 4, 5, or 6, of the areas of the PNS recited above. In some embodiments, the CRE, CRM, minimal/proximal promoter or promoter of the present invention is active in the majority of the areas in the PNS, i.e. at least 4, at least 5, or all 6 of the 6 areas of the PNS recited above.

The CREs, minimal/proximal promoters or promoters of the present invention can be active in a combination of one or more of the various areas of the PNS and the CNS described above.

In some embodiments, it may be desirable that the CRE, minimal/proximal promoter or promoter of the present invention shows predominant activity in one area of the CNS, suitably in one area of the brain, or in one area of the spinal cord. Suitably, it may be desirable that the CRE, minimal/proximal promoter or promoter of the present invention shows activity in one area of the brain but no, or only minimal, activity in the rest of the brain, spinal cord, CNS or PNS. Suitably, it may be desirable that the CRE, minimal/proximal promoter or promoter of the present invention shows activity in one area of the spinal cord but no, or only minimal, activity in the rest of the spinal cord, brain, CNS or PNS. In some embodiments, the CRE, minimal/proximal promoter or promoter of the present invention is active in only one area of the areas of the brain or spinal cord recited above.

In some embodiments, it may be desirable that the CRE, minimal/proximal promoter or promoter of the present invention shows predominant activity in one area of the PNS. Suitably, it may be desirable that the CRE, minimal/proximal promoter or promoter of the present invention shows activity in one area of the PNS but no, or only minimal, activity in the brain, CNS or rest of the PNS. In some embodiments, the CRE, minimal/proximal promoter or promoter of the present invention is active in only one area of the areas of the PNS recited above.

The CREs, minimal/proximal promoters or promoters of the present invention can be active in various cells of the nervous system. The cells may be CNS cells and/or PNS cells.

The CREs, minimal/proximal promoters or promoters of the present invention can be active in various cells of the PNS. The predominant cell types in the PNS are neurones, satellite cells and Schwann cells. Other cell types may be present, particularly in inflammatory condition. In some embodiments, it may be desirable for the promoter to be active in many different cell types. In some embodiments, the CRE, CRM, minimal/proximal promoter or promoter of the present invention is active in substantially all cells of the PNS (e.g. neurones, satellite cells and Schwann cells). In some embodiments, the CRE, CRM, minimal/proximal promoter or promoter of the present invention is active in at least two PNS cell types from the PNS cell types listed above, such as neurones and satellite cells.

The CREs, minimal/proximal promoters or promoters of the present invention can be active in various cells of the CNS. The predominant cell types in the CNS are neurones, astrocytes, oligodendrocytes, microglia, and ependymal cells. Other cell types may be present, particularly in inflammatory condition. In some embodiments, it may be desirable for the promoter to be active in many different cell types. In some embodiments, the CRE, CRM, minimal/proximal promoter or promoter of the present invention is active in substantially all cells of the CNS (e.g. neurones, astrocytes, oligodendrocytes, microglia, ependymal cells). In some embodiments, the CRE, CRM, minimal/proximal promoter or promoter of the present invention is active in at least four CNS cell types from the CNS cell types listed above, such as neurones, astrocytes, microglia and oligodendrocytes. In some embodiments, the CRE, CRM, minimal/proximal promoter or promoter of the present invention is active in at least three CNS cell types from the CNS cell types listed above, such as neurones, astrocytes and oligodendrocytes. In some embodiments, the CRE, CRM, minimal/proximal promoter or promoter of the present invention is active in at least two CNS cell types from the CNS cell types listed above, such as neurones and astrocytes.

The CREs, minimal/proximal promoters or promoters of the present invention may be active in any combinations of cells of the PNS and/or the CNS as described above. The CREs, minimal/proximal promoters or promoters of the present invention may be active in interneurons.

In some embodiments, it may be desirable for the promoter to be active in a limited number of CNS cell types, or in not more than one CNS cell type. In some embodiments, the CRE, CRM minimal/proximal promoter or promoter of the present invention is active in no more than 4, 3, 2 or 1 of CNS cell types from the CNS cell types listed above. In some embodiments, the CRE, CRM, minimal/proximal promoter or promoter of the present invention is active in no more than three CNS cell types from the CNS cell types listed above, such as neurones, astrocytes, and oligodendrocytes. In some embodiments, the CRE, CRM, minimal/proximal promoter or promoter of the present invention is active in no more than two CNS cell types from the CNS cell types listed above, such as neurones, and astrocytes. In some embodiments, the CRE, CRM, minimal/proximal promoter or promoter of the present invention is active in only one CNS cell type from the CNS cell types listed above, such as neurones.

In some embodiments, it may be desirable for the promoter to be active in a limited number of PNS cell types, or in not more than one PNS cell type. In some embodiments, the CRE, CRM minimal/proximal promoter or promoter of the present invention is active in no more than 2 or 1 of PNS cell types from the PNS cell types listed above.

In some embodiments, the CRE, CRM, minimal/proximal promoter or promoter of the present invention is active in no more than two PNS cell types from the PNS cell types listed above, such as neurones, and satellite cells. In some embodiments, the CRE, CRM, minimal/proximal promoter or promoter of the present invention is active in only one PNS cell type from the PNS cell types listed above, such as neurones.

In some embodiments, the CRE, CRM, minimal/proximal promoter or promoter of the present invention is active in specific subtypes of CNS cell, such as for example spiny GABAergic projection neurones (found in the striatum), dopaminergic neurones (found in the substantia nigra), pyramidal neurones (found in layers 3, 5 and 6 of the cerebral cortex), hippocampal neurones (found in the hippocampus), purkinje cells (found in the cerebellum), lateral tuberal nuclei neurones (found in the hypothalamus/thalamus). In some specifically preferred embodiments, the CRE, CRM, minimal/proximal promoter or promoter of the present invention is active in any or all of the neuronal subtypes listed above. In some preferred embodiments, the CRE, CRM, minimal/proximal promoter or promoter of the present invention is active in any or all of the neuronal subtypes listed above but not in other CNS or PNS cell types or subtypes. In some preferred embodiments, the CRE, CRM, minimal/proximal promoter or promoter of the present invention is active in any or all of the neuronal subtypes listed above and in other CNS cell types or other CNS cell subtypes.

In some embodiments, the CRE, CRM, minimal/proximal promoter or promoter of the present invention is active in GABAergic or glutamatergic neurones.

In some embodiments, the CRE, CRM, minimal/proximal promoter or promoter of the present invention is active in specific subtypes of PNS cell, such as for example motor neurones, PNS sympathetic neurones, PNS enteric neurones and sensory neurones. In some specifically preferred embodiments, the CRE, CRM, minimal/proximal promoter or promoter of the present invention is active in any or all of the PNS neuronal subtypes listed above but not in other nervous system cell types or subtypes. In some embodiments, the CRE, CRM, minimal/proximal promoter or promoter of the present invention is active in any or all of the PNS neuronal subtypes listed above and in other CNS and/or PNS cell types or other CNS cell subtypes.

In some embodiments, the CRE, CRM, minimal/proximal promoter or promoter of the present invention is active in a specific type of CNS cell or a subtype of CNS cell, and in a specific area of the CNS, suitably a specific area of the brain. In some embodiments, the CRE, CRM, minimal/proximal promoter or promoter of the present invention is active in a specific type of PNS cell or a subtype of PNS cell, and in a specific area of the PNS. In some embodiments, the CRE, CRM, minimal/proximal promoter or promoter of the present invention is active in one or more specific type or subtype of CNS and/or PNS cells, and in a specific area of the CNS and/or PNS.

The CRE, CRM, minimal/proximal promoter or promoter of the present invention may or may not be active in tissues outside the NS. Non-limiting examples of tissues outside the NS are: the heart, the liver, the kidney, skeletal muscles and the spleen. Suitably, in some embodiments, the CRE, CRM, minimal/proximal promoter or promoter of the present invention is not or is minimally active in tissues or cells outside of the NS. In some embodiments, the CRE, CRM, minimal/proximal promoter or promoter of the present invention is more highly expressed in the NS as compared to expression in tissues or cells outside of the NS. Suitably, the CRE, CRM, minimal/proximal promoter or promoter of the present invention is active in no more than 1, 2, 3, or 4 tissues out of the tissues outside of the NS described above, suitably in systemic delivery such as IV delivery.

Suitably, in some embodiments, it may be desirable for the CRE, CRM, minimal/proximal promoter or promoter of the present invention to be active in the NS but to also have activity in other tissues outside of the NS. Suitably, the CRE, CRM, minimal/proximal promoter or promoter of the present invention may be active in at least 1, 2, 3, 4 or 5 of the tissues outside of the NS described above, suitably in systemic delivery such as IV delivery.

Expression driven by a promoter of the present invention in a desired tissue or cell may be for a period of at least 1 hour, 2, hours, 3 hours, 4 hours, 5 hours, 6 hours, 7 hours, 8 hours, 9 hours, 10 hours, 1 1 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 15 years, 20 years, 30 years, 40 years, 50 years, 60 years, 70 years, 80 years, 90 years, 100 years. Expression driven by a promoter of the present invention in a desired tissue or cell may be for a period of more than 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 20 years, 30 years, 40 years, 50 years, 60 years, 70 years, 80 years, 90 years, 100 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, 5-10 years, 10-15 years, 15-20 years, 20-30 years, 30-40 years, 40-50 years, 50-60 years, 60-70 years, 80-90 years or 90-100 years.

In a further aspect of the invention, there is provided an expression cassette comprising a synthetic NS-specific promoter of any aspect of the present invention operably linked to a sequence encoding an expression product. Suitably, the expression product is a gene, e.g. a transgene. In some embodiments, the expression product is a therapeutic expression product.

In a further aspect, there is provided a vector comprising a synthetic NS-specific promoter or an expression cassette according to the present invention. In some embodiments, the vector is an expression vector. In some embodiments the vector is a viral vector. In some embodiments, the vector is a gene therapy vector, suitably an AAV vector, an adenoviral vector, a retroviral vector, a herpes simplex vector or a lentiviral vector. Lentiviral vectors have been extensively used as a gene transfer tool in the CNS and are known to be able to successfully transduce neurones, astrocytes and oligodendrocytes (Jakobsson and Lundberg, 2006). They are beneficial as they have relatively large cloning capacity and because viral genes are not expressed. A particularly preferred lentiviral vector system is based on HIV-1 (Jakobsson and Lundberg, 2006). Herpes simplex viral vectors and adenoviral vectors also show potential for use in as a gene transfer tool in CNS as they show successful transduction of CNS cells but are less preferred due to their toxicity.

AAV vectors have been extensively discussed in the art. AAV vectors are of particular interest as AAV vectors do not typically integrate into the genome and do not elicit immune response. AAV serotypes 1, 2, 4, 5, 8, 9, rh10, DJ8, 2i8, 2.5, 2i8G9 and 2g9 (AAV1, AAV2, AAV4, AAV5, AAV8, AAV9, AAVrh10, AAVDJ8, AAV2i8, AAV2.5, AAV2i8G9 and AAV2g9) have been noted to achieve efficient transduction in the NS. Therefore, AAV1, AAV2, AAV4, AAV5, AAV8, AAV9, AAVrh10, AAVVDJ8, AAV2i8, AAV2.5, AAV2i8G9, AAV2g9 and derivatives thereof are particularly preferred AAV serotypes. In some embodiments, AAV9 is particularly preferred AAV vector. In other embodiments, AAV2g9 is a particularly preferred AAV vector (WO2014/144229). In yet other embodiments, a particularly preferred AAV vector is AAVDJ8. In some embodiments, AAVrh10 is particularly preferred AAV vector. Suitably an AAV vector comprises a viral genome which comprises a nucleic acid sequence of the present invention positioned between two inverted terminal repeats (ITRs). WO2019/028306, for example discloses various wild type and modified AAV vectors that can be used in the CNS. In one embodiment, the AAV vector is capable of penetrating the blood brain barrier following delivery of the AAV vector. In one embodiment, AAV vectors of the present invention are recombinant AAV viral vectors which are replication defective, lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ or an organism. Suitably AAV vectors for use herein comprise a virus that has been reduced to the minimum components necessary for transduction of a nucleic acid payload or cargo of interest. In this manner, AAV vectors are engineered as vehicles for specific delivery while lacking the deleterious replication and/or integration features found in wild-type viruses. In one embodiment, the AAV particle of the present invention is an scAAV. In another embodiment, the AAV particle of the present invention is an ssAAV. Methods for producing and/or modifying AAV particles are disclosed extensively in the art (see e.g. WO2000/28004; WO2001/23001; WO2004/112727; WO 2005/005610 and WO 2005/072364, which are incorporated herein by reference). In one embodiment the AAV vector comprises a capsid that allows for blood brain barrier penetration following intravascular (e.g. intravenous or intraarterial) administration (see e.g. WO2014/144229, which discusses, for example, capsids engineered for efficient crossing of the blood brain barrier, e.g. capsids or peptide inserts including VOY101, VOY201, AAVPHP.N, AAVPHP.A, AAVPHP.B, PHP.B2, PHP.B3, G2A3, G2B4, G2B5, PHP.S, and variants thereof).

Methods of making AAV vectors are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO 1999014354, WO1999/015685, WO1999/047691, WO2000/055342, WO2000/075353 and WO2001/023597; Methods In Molecular Biology, ed. Richard, Humana Press, N J (1995); O'Reilly et al, Baculovirus Expression Vectors, A Laboratory Manual, Oxford Univ. Press (1994); Samulski et al., J Fir. 63:3822-8 (1989); Kajigaya et al, Proc. Nat′l. Acad. Sci. USA 88: 4646-50 (1991); Ruffing et al., J. Vir. 66:6922-30 (1992); Kimbauer et al, Vir., 219:37-44 (1996); Zhao et al, Vir. 272: 382-93 (2000); the contents of each of which are herein incorporated by reference. Viral replication cells commonly used for production of recombinant AAV viral particles include but are not limited to HEK293 cells, COS cells, HeLa cells, KB cells, and other mammalian cell lines.

It is to be understood that a viral expression system will further be modified to include any necessary elements required to complement a given viral vector during its production using methods described herein. For example, in certain embodiment, the nucleic acid cassette is flanked by terminal repeat sequences. In one embodiment, for the production of rAAV vectors, the AAV expression system will further comprise at least one of a recombinant AAV plasmid, a plasmid expressing Rep, a plasmid expressing Cap, and an adenovirus helper plasmid. Complementary elements for a given viral vector are well known the art and a skilled practitioner would be capable of modifying the viral expression system described herein accordingly.

A viral expression system for manufacturing an AAV vector (e.g., an AAV expression system) could further comprise Replication (Rep) genes and/or Capsid (Cap) genes in trans, for example, under the control of an inducible promoter. Expression of Rep and Cap can be under the control of one inducible promoter, such that expression of these genes are turned “on” together, or under control of two separate inducible promoters that are turned “on” by distinct inducers. On the left side of the AAV genome (towards the 5′ end) there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the inverted terminal repeats (ITR) in the self-priming act and cleave at the designated terminal resolution site, within the hairpin. They are necessary for the AAVS1-specific integration of the AAV genome. All four Rep proteins were shown to bind ATP and to possess helicase activity. The right side (towards the 3′ end) of a positive-sensed AAV genome encodes overlapping sequences of three capsid proteins, VP1, VP2 and VP3, which are operably linked to one promoter, designated p40. The cap gene produces an additional, non-structural protein called the Assembly-Activating Protein (AAP). This protein is produced from open reading frame 2 (ORF2) and is essential for the capsid-assembly process. Necessary elements for manufacturing AAV vectors are known in the art, and can further be reviewed, e.g., in U.S. Pat. Nos. 5,478,745A; 5,622,856A; 5,658,776A; 6,440,742B1; 6,632,670B1; 6,156,303A; 8,007,780B2; 6,521,225B1; 7,629,322B2; 6,943,019B2; 5,872,005A; and U.S. Patent Application Numbers US 2017/0130245; US20050266567A1; US20050287122A1; the contents of each are incorporated herein by reference in their entireties.

In one embodiment, the cells for producing an AAV vector are cultured in suspension. In another embodiment, the cells are cultured in animal component-free conditions. The animal component-free medium can be any animal component-free medium (e.g., serum-free medium) compatible with a given cell line, for example, HEK293 cells. Any cell line known in the art to be capable of propagating an AAV vector can be used for AAV production using methods described herein. Exemplary cell lines that can be used to generate an AAV vector include, without limitation, HEK293, CHO, Cos-7, and NSO.

In one embodiment, a cell line for producing an AAV vector stably expresses any or all of the components required for AAV vector production, e.g., Rep, Cap, VP1, etc. In one embodiment, a cell line for producing an AAV vector transiently expresses any or all of the components required for AAV vector production, e.g., Rep, Cap, VP1, etc.

In the event that a cell line for producing AAV vectors does not stably or transiently express rep or cap, these sequences are to be provided to the AAV expression system. AAV rep and cap sequences may be provided by any method known in the art. Current protocols typically express the AAV rep and/or cap genes on a single plasmid. The AAV replication and packaging sequences need not be provided together, although it may be convenient to do so. The AAV rep and/or cap sequences may be provided by any viral or non-viral vector. For example, the rep and/or cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., arc stably integrated into the cell as extra-chromosomal elements, designated as an “EBV based nuclear episome,” see Margolski, Curr. Top. Microbial. Immun. 158:67 (1992)).

Typically, the AAV rep/cap sequences will not be flanked by the TRs, to prevent rescue and/or packaging maintain of these sequences.

A viral expression system for manufacturing a lentivirus using methods described herein would further comprise long terminal repeats (LTRs) flanking the nucleic acid cassette. LTRs are identical sequences of DNA that repeat hundreds or thousands of times at either end of retrotransposons or proviral DNA formed by reverse transcription of retroviral RNA. The LTRs mediate integration of the retroviral DNA via an LTR specific integrase in the host chromosome. LTRs and methods for manufacturing lentiviral vectors are further described, e.g., in U.S. Pat. No. 7,083,981B2; 6,207,455B1; 6,555,107B2; 8,349,606B2; 7,262,049B2; and U.S. Patent Application Numbers US20070025970A1; US20170067079A1; US20110028694A1; the contents of each are incorporated herein by reference in their entireties.

A viral expression system for manufacturing an adenovirus using methods described herein would further comprise identical Inverted Terminal Repeats (ITR) of approximately 90-140 base pairs (exact length depending on the serotype) flanking the nucleic acid cassette. The viral origins of replication are within the ITRs exactly at the genome ends. The adenovirus genome is a linear double-stranded DNA molecule of approximately 36000 base pairs. Often, adenoviral vectors used in gene therapy have a deletion in the E1 region, where novel genetic information can be introduced; the E1 deletion renders the recombinant virus replication defective. ITRs and methods for manufacturing adenovirus vectors are further described, e.g., in U.S. Pat. No. 7,510,875B2; 7,820,440B2; 7,749,493B2; 7,820,440B2; U.S. Ser. No. 10/041,049B2; International Patent Application Numbers WO2000070071A1; and U.S. patent Application 5 Numbers WO2000070071A1; US20030022356A1; US20080050770A1 the contents of each are incorporated herein by reference in their entireties.

In one embodiment, the viral expression system can be a host cell, such as a virus, a mammalian cell or an insect cell. Exemplary insect cells include but are not limited to Sf9, Sf21, Hi-5, and S2 insect cell lines. For example, a viral expression system for manufacturing an AAV vector could further comprise a baculovirus expression system, for example, if the viral expression system is an insect cell. The baculovirus expression system is designed for efficient large-scale viral production and expression of recombinant proteins from baculovirus-infected insect cells. Baculovirus expression systems are further described in, e.g., U.S. Pat. No. 6,919,085B2; 6,225,060B1; 5,194,376A; the contents of each are incorporated herein by reference in their entireties.

In another embodiment, the viral expression system is a cell-free system. Cell-free systems for viral vector production are further described in, for example, Cerqueira A., et al. Journal of Virology, 2016; Sheng J., et al. The Royal Society of Chemistry, 2017; and Svitkin Y. V., and Sonenberg N. Journal of Virology, 2003; the contents of which are incorporated herein by reference in their entireties.

Viral vectors produced in a cell can be released (i.e. set free from the cell that produced the vector) using any standard technique. For example, viral vectors can be released via mechanical methods, for example microfluidization, centrifugation, or sonication, or chemical methods, for example by addition of lysis buffers and detergents. Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). As described herein in the examples, viral vectors can be released from the cell via sonication and recovered via purification of clarified lysate using column chromatography.

In some embodiments the vector is a non-viral vector, for example using cationic polymers or cationic lipids, as is known in the art. Various non-viral vectors are discussed in Selene Ingusci et al. (Gene Therapy Tools for Brain Diseases. Front. Pharmacol. 10:724. doi: 10.3389).

In a further aspect, there is provided a virion (viral particle) comprising a vector, suitably a viral vector, according to the present invention. In some embodiments the virion is an AAV virion.

In a further aspect, there is provided a pharmaceutical composition comprising a synthetic NS-specific promoter, expression cassette, vector or virion according to the present invention.

For example, AAV vector particles may be prepared as pharmaceutical compositions. It will be understood that such compositions necessarily comprise one or more active ingredients and, most often, a pharmaceutically acceptable excipient.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In a further aspect, there is provided a synthetic NS-specific promoter, expression cassette, vector, virion or pharmaceutical composition according to the present invention for use as medicament.

In a further aspect, there is provided a synthetic NS-specific promoter, expression cassette, vector, virion or pharmaceutical composition according to the present invention for use in therapy, i.e. the prevention or treatment of a medical condition or disease.

In a further aspect, there is provided a synthetic NS-specific promoter, NS-specific cis-regulatory element (CRE), CRM, promoter element, expression cassette, vector, virion or pharmaceutical composition as described herein for use in the manufacture of a pharmaceutical composition for the treatment of a medical condition or disease. Exemplary medical conditions or diseases relevant to the present aspect are discussed below.

Suitably the medical condition or disease is associated with aberrant gene expression, optionally aberrant gene expression in the nervous system, suitably CNS and/or PNS tissue or cells. Suitably the use is for gene therapy, preferably for use in the treatment of a disease involving aberrant gene expression. Suitably, the medical condition or disease involving aberrant gene expression may be a disease of the CNS and/or the PNS. Suitably, the medical condition or disease may be a single gene disorder of the CNS and/or the PNS. Suitably the gene therapy involves expression of a therapeutic expression product in CNS and/or the PNS cells or tissue. Suitably, the expression product may be a wild type allele of a faulty gene, an expression product preventing the expression of a faulty gene or an expression product counteracting the detrimental effects of a faulty gene. Exemplary medical conditions or diseases relevant to the present aspect are discussed below.

In one embodiment, the medical condition or disease is Huntington's disease.

In a further aspect, there is provided a cell comprising a synthetic NS-specific promoter, expression cassette, vector, or virion of the present invention. In some embodiments the cell is a mammalian cell, optionally a human cell. Suitably, the cell is a CNS or a PNS cell. Suitably the cell may be a neurone, interneuron, or a neuronal supporting cell, e.g., an astrocyte, an oligodendrocyte, ependymal cell or a microglial cell. Suitably the cell may be a human neurone, astrocyte, oligodendrocyte, ependymal cell, microglial cell, Schwann cell or a satellite cell. The synthetic NS-specific promoter can be episomal or can be in the genome of the cell.

In one embodiment, the cell is a human neurone, a human astrocyte, or a human oligodendrocyte.

In a further aspect, there is provided a method for producing an expression product, the method comprising providing a synthetic NS-specific expression cassette, vector or a virion of the present invention in NS cells or tissue and expressing the gene of interest present in the synthetic NS-specific expression cassette, vector or virion. The method can be in vitro or ex vivo, or it can be in vivo.

In a further aspect, there is provided a method of expressing a therapeutic transgene in a NS cell, the method comprising introducing into the NS cell a synthetic NS-specific expression cassette, vector or virion as described herein and expressing the expression product (e.g. gene of interest) present in the synthetic NS-specific expression cassette, vector or virion.

The NS cell may be a cell of the CNS (a neuron, an astrocyte, an oligodendrocyte, an ependymal cell or a microglial cell) or the PNS (a neuron, a Schwann cell or a satellite cell) The NS cell may be a neuron, an interneuron, or a neuronal supporting cell, for example, an astrocyte, an oligodendrocyte, an ependymal cell, a microglial cell, a Schwann cell or a satellite cell.

In one embodiment, the CNS cell is a neuron, an astrocyte, or an oligodendrocyte.

In a further aspect, there is provided a method of therapy of a subject, preferably a human in need thereof, the method comprising:
administering to the subject an expression cassette, vector, virion or a pharmaceutical composition as described herein, which comprises a sequence encoding a therapeutic product operably linked to a promoter according to the present invention; and
expressing a therapeutic amount of the therapeutic product in the NS of said subject.

In one embodiment, the therapeutic amount of the therapeutic product is expressed in the CNS. In one embodiment, the therapeutic amount of the therapeutic product is expressed in the PNS. In one embodiment, the therapeutic amount of the therapeutic product is expressed in the CNS and the PNS.

Suitably the method is for the treatment, prophylaxis, palliation or amelioration of neurological diseases and/or disorders. Exemplary medical conditions or diseases relevant to the present aspect are discussed below.

Suitable methods of administration may be enteral (e.g. oral, sublingual, and rectal) or parenteral (e.g. injection) including intravenous, intraarterial, intracerebroventricular, intracranial, intramuscular, subcutaneous, intra-articular, intrathecal, and intradermal injections. Preferred administration methods are intravenous, intraarterial, intracerebroventricular, intracranial and intrathecal injection.

In some embodiments the method comprises introducing into the NS of the subject an expression cassette, vector, virion or a pharmaceutical composition as described herein, which comprises a gene encoding a therapeutic product. A particular difficulty with introducing an expression cassette, vector, virion or a pharmaceutical composition in the CNS is the blood brain barrier. The blood brain barrier is a semipermeable border of endothelial cells that prevents certain chemicals and molecules in the bloodstream from crossing into the extracellular fluid of the central nervous system. In animal studies, this obstacle has been overcome by injection directly into the brain of the animal, such as intracranial injection, suitably intracerebroventricular (ICV) injection (see e.g. Keiser et al., Curr. Protoc. Mouse Biol. 2018 Dec.; 8(4):e57). This method of administration can be disadvantageous for gene therapy in humans as it is difficult to perform and can be dangerous for the subject.

Instead, in a gene therapy setting in human, it is preferred that the expression cassette as described herein is introduced into the NS by intravenous, intranasal or intraarterial (e.g. intra-carotid) administration of a viral vector comprising the expression cassette. Suitably, the viral vector is an AAV vector. Intravenous or intraarterial administration of some serotypes of AAV allows penetration of the AAV vectors into the NS. Minimal expression in non-NS tissues and cells is expected due to the NS-specificity of the synthetic NS-specific promoters according to the present invention. Furthermore, it is expected that with the development of improved AAV capsids specifically for CNS-penetration, penetration of AAV vectors will be improved. Intravenous or intraarterial administration is safer and less invasive than intracranial administration, while still allowing penetration through the blood brain barrier.

In some embodiments, the AAV vector is administered directly into the CSF. For example, the AAV vector may be administered via intrathecal (IT) or intracerebroventricular (ICV) administration. “Intrathecal” administration is used to mean intrathecal injection, i.e., administration into the cerebrospinal fluid at any level of the cerebrospinal axis, including injection into the ventricles. This is an injection into the subarachnoid space via the spinal meninges so that the injection reaches the cerebrospinal fluid (CSF). Because injection into the brain or spinal cord requires complex brain surgery, intrathecal delivery methods are considered less invasive than injecting into the CNS tissue itself. Intrathecal delivery can be performed without the need for a specialized center for brain injection.

In one embodiment, the AAV vector is administered by lumbar administration. The intralumbar intrathecal administration is preferably at a position selected from the group consisting of L4 to L5, L3 to L4, L1 to L2, and L2 to L3 spinal cord segments. Most preferably, the AAV vector is administered by intrathecal injection between L4 and L5. In one embodiment, the AAV vector is administered in a cisterna magna. In one embodiment, the AAV vector is administered in a cisterna magna and/or via lumbar administration. In another embodiment, administration into the cisterna is performed in combination with lumbar administration, whereby administration into the cisterna is performed before, simultaneously with, or after lumbar administration. In this embodiment, the lumbar administration is in the intrathecal region. The “large cisterna” or “cerebellar medullary tubule” is one of the three main openings in the subarachnoid space between the arachnoid and pial membranes of the meninges around the brain. The openings are collectively referred to as a tank. The cisterna is located between the cerebellum and the dorsal surface of the medulla. Cerebrospinal fluid produced in the fourth ventricle is drained into the cisterna magna through the lateral and median ports.

In some embodiments, the AAV vector may be administered in combination with a means for blood brain barrier disruption. Suitably to allow the AAV vector to penetrate the blood brain barrier more easily.

Suitably, the medical condition or disease is a medical condition or disease of the CNS and/or the PNS.

Suitably, the medical condition or disease is a medical condition or disease of the PNS. Suitably, the medical condition or disease may be selected from, for example: accessory nerve disorder, alcoholic polyneuropathy, anestesia dolorosa, anti-MAG peripheral neuropathy, autoimmune autonomic ganglionopathy, autonomic dysreflexia, autonomic neuropathy, axillary nerve dysfunction, axillary nerve palsy, Charcot-Marie-Tooth disease, chemotherapy-induced peripheral neuropathy, chronic solvent-induced encephalopathy, CMV polyradiculomyelopathy, congenital insensitivity to pain with anhidrosis, denervation, diabetic neuropathy, dysautonomia, erythromelalgia, facial nerve paralysis, familial dysautonomia, Guillain-Barre syndrome, hereditary sensory and autonomic neuropathy, Horner's syndrome, nerve compression syndrome, nerve injury, neurapraxia, neuritis, orthostatic hypotension, orthostatic intolerance, paroxysmal sympathetic hyperactivity, peripheral mononeuropathy, peripheral neuropathy, Piriformis syndrome, plexopathy, polyneuropathy, postural orthostatic tachycardia syndrome, primary autonomic failure, Pronator teres syndrome, proximal diabetic neuropathy, Pudendal nerve entrapment, pure autonomic failure, quadrilateral space syndrome, radial nerve dysfunction, radial neuropathy, radiation-induced lumbar plexopathy, radiculopathy, sciatica, small fiber peripheral neuropathy, thoracic outlet syndrome, Ulnar neuropathy, vasculitic neuropathy, Villaret's syndrome, Wartenberg's syndrome and Winged scapula.

Suitably, the medical condition or disease is a medical condition or disease of the CNS, e.g. a neurological disease and/or disorder. Suitably, the medical condition or disease may be selected from, for example: dopamine transporter deficiency syndrome, an attention deficit/hyperactivity disorder (ADHD), bipolar disorder, epilepsy, multiple sclerosis, tauopathies, Alzheimer's disease, Huntington's disease, Parkinson's disease, Krabbe's disease, adrenoleukodystrophy, motor neurone disease (MND), Primary lateral sclerosis (PLS), spinal muscular atrophy (SMA), Kennedy's disease, cerebral palsy, Gaucher disease, Tay Sachs disease, Rett syndrome, Sandhoff disease, Charcot-Marie-Tooth disease, Angelman syndrome, Transmissible spongiform encephalopathies (TSEs) (including Creutzfeldt-Jakob disease (CJD), kuru, fatal familial insomnia (FFI), and Gerstmann-Straussler-Scheinker disease (GSS)), Late infantile neuronal ceroid lipofuscinosis, Mucopolysaccharidosis IIIA, Mucopolysaccharidosis IIIB, leukodystrophies (such as metachromatic leukodystrophy, Krabbe disease, adrenoleukodystrophy, Pelizaeus-Merzbacher disease (PMD), Canavan disease, Childhood Ataxia with Central Nervous System Hypomyelination or CACH, Alexander disease, Refsum disease, and cerebrotendinous xanthomatosis), heritable lysosomal storage diseases such as Niemann-Pick disease type C1 or gangliosidosis, and/or neuronal ceroid lipofuscinoses such as Batten disease, progressive supranuclear palsy (PSP), corticobasal syndrome, and brain or spinal cord tumors or cancer (including astrocytomas and glioblastomas) or various forms of CNS cancer, such as primary CNS lymphoma, Transfer or secondary brain tumor, primary spinal cord tumors, Familial Periodic Paralyses, Friedreich's Ataxia, telangiectasia ataxia, spinocerebellar ataxia type 1, type 2, and type 3, dopa-responsive dystonia Fragile X chromosome syndrome, Frontotemporal dementia, Frontotemporal dementia with motor neuron disease (FTD-MND), Hereditary spastic paraplegia (HSP) or familial spastic paraparesis (FSP), spinal cord injury (SCI), traumatic brain injury (TBI), stroke, spinal cord infarction, psychiatric diseases (severe depression, obsessive compulsive disorder), Giant axonal neuropathy (GAN), pain (including cancer pain, Glossopharyngeal neuralgia (GN) and Trigeminal neuralgia (TN)), Locked-in syndrome, Neuromyelitis optica (NMO), Olivopontocerebellar atrophy (OPCA), inherited and non-inherited forms of ataxia (such as the hereditary spinocerebellar ataxia known as Machado-Joseph disease) and multiple system atrophy (MSA), Schilder's disease. In one embodiment, the medical condition or disease is Huntington's disease.

Suitably, the sequence encoding a therapeutic product may be one of the genes selected from the group consisting of: NPC1, EAAT2, NPY, CYP46A1, GLB1, APOE (or APOE2), HEX, CLN1, CLN2, CLN3, CLN4, CLN5, CLN6, SUMF1, DCTN1, PRPH, SOD1, SMN, NEFH, GBA, IDUA, NAGLU, GUSB, ARSA, MANB, AADC, GDNF, NTN, ASP, MECP2, PTCHD1, GJB1, UBE3A, HEXA, FXN and MOG. In some embodiments, the sequence encoding a therapeutic product may be one of the genes selected from the group consisting of aspartyl glucosaminidase, α-galactosidase A, palmitoyl protein thioesterase, tripeptidyl peptidase, lysosomal transmembrane protein, cysteine transporter, acid ceramidase, acid α-L-Fucosidase, protective protein/cathepsin A, acid β-glucosidase or glucocerebrosidase, acid β-galactosidase, iduronic acid-2-sulfatase, α-L-iduronidase, galactocerebrosidase, acid α-mannosidase, acid β-mannosidase, Arylsulfatase B, arylsulfatase A, Nacetylgalactosamine-6-sulfate sulfatase, acid β-galactosidase, N-acetylglucose Samine-1-phosphotransferase, acid sphingomyelinase, NPC-1, acid α-glucosidase, β-hexosaminidase B, heparan N-sulfatase, α-N-acetylglucosaminidase, acetyl-CoA: α-glucosaminide NAcetyltransferase, N-acetylglucosamine-6-sulfate sulfatase, α-N-acetylgalactosaminidase, α-N acetylgalactosaminidase, α-neuramidase, β-glucuronidase, R-hexosaminidase A, acid lipase, Neurotrophic factors such as nerve growth factor (NGF), neurotrophin-3 (NT-3), neurotrophin-4/5 (NT-4/5), brain-derived neurotrophic factor (BDNF), brain dopamine Neurotrophic factor (CDNF), glial-derived neurotrophic factor (GDNF), ciliary neurotrophic factor (CNTF), insulin-like growth factor (IGF-1), Huntington gene (also known as HTT, HD, and IT15).

In one embodiment, the sequence encoding a therapeutic product is the CYP46A1 gene.

Exemplary gene products and medical condition or diseases of the CNS diseases treated with an AAV vector (either as treatment or prophylaxis) as disclosed herein are GDNF or IGF-1 for the treatment of ALS, GDNF or IGF for the treatment of SMA-1, miRNA for down-regulation of incomplete spinal motor neuron survival protein (SMN) gene for treatment of SMA, allele-specific down-regulation for treatment of Huntington's disease or down-regulation of both alleles of HTT, GDNF for the treatment of Huntington's disease, GDNF for the treatment of MSA, α-galactosidase A for the treatment of Fabry disease, lysosomal transmembrane protein for the treatment of pediatric Batten's disease (CNL3), Gaucher disease type 1, type 2 And acid β-glucosidase or glucocerebrosidase for treatment of type 3, iduronic acid-2-sulfatase for treatment of Hunter syndrome, acid α-glucosidase for treatment of Pompe disease, treatment of Sanfilippo syndrome type A Selected from the group consisting of heparan N-sulfatase for A and N-acetylglucosaminidase for the treatment of San Filippo syndrome type B.

Additionally, or alternatively, the therapeutic product may be an antibody, antibody fragment or anti-body like scaffold protein.

Additionally, or alternatively, the therapeutic product may be a gene editing system (such as a CRISPR-Cas9 system, TALEN, ZFN, etc.) directed to the disease allele.

Additionally, or alternatively, the therapeutic product may be one or more modulatory polynucleotides, e.g., RNA or DNA molecules as therapeutic agents. For example the modulatory polynucleotide may be a miRNA or siRNA. Target genes may be any of the genes associated with any neurological disease such as, but not limited to, those listed herein. For example, siRNA duplexes or encoded dsRNA can reduce or silence target gene expression in NS cells, thereby ameliorating symptoms of neurological disease. In one non-limiting example, the target gene is huntingtin (HTT). In another non-limiting example the target gene is microtubule-associated protein tau (MAPT). In one embodiment, the therapeutic product is a modulatory polynucleotide targeting the HTT gene.

In a further aspect, there is provided a method of expressing an expression product in the NS, the method comprising introducing into the NS cell an expression cassette comprising a synthetic NS-specific promoter comprising or consisting of SEQ ID NO: 1 operably linked to the expression product. Suitably, the expression product is expressed in neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 2 operably linked to the expression product. Suitably, the expression product is expressed in neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 3 operably linked to the expression product. Suitably, the expression product is expressed in excitatory neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 4 operably linked to the expression product. Suitably, the expression product is expressed in excitatory neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 5 operably linked to the expression product. Suitably, the expression product is expressed in neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 6 operably linked to the expression product. Suitably, the expression product is expressed in neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 7 operably linked to the expression product. Suitably, the expression product is expressed in neurones, oligodendrocytes and astrocytes.

In a further aspect, there is provided a method of expressing an expression product in the NS, the method comprising introducing into the NS cell an expression cassette comprising a synthetic NS-specific promoter comprising or consisting of SEQ ID NO: 8 operably linked to the expression product. Suitably, the expression product is expressed in neurones, oligodendrocytes and astrocytes.

In a further aspect, there is provided a method of expressing an expression product in the NS, the method comprising introducing into the NS cell an expression cassette comprising a synthetic NS-specific promoter comprising or consisting of SEQ ID NO: 9 operably linked to the expression product. Suitably, the expression product is expressed in neurones, oligodendrocytes and astrocytes.

In a further aspect, there is provided a method of expressing an expression product in the NS, the method comprising introducing into the NS cell an expression cassette comprising a synthetic NS-specific promoter comprising or consisting of SEQ ID NO: 10 operably linked to the expression product. Suitably, the expression product is expressed in neurones, oligodendrocytes and astrocytes.

In a further aspect, there is provided a method of expressing an expression product in the NS, the method comprising introducing into the NS cell an expression cassette comprising a synthetic NS-specific promoter comprising or consisting of SEQ ID NO: 11 operably linked to the expression product. Suitably, the expression product is expressed in neurones, oligodendrocytes and astrocytes.

In a further aspect, there is provided a method of expressing an expression product in the NS, the method comprising introducing into the NS cell an expression cassette comprising a synthetic NS-specific promoter comprising or consisting of SEQ ID NO: 12 operably linked to the expression product. Suitably, the expression product is expressed in neurones, oligodendrocytes and astrocytes.

In a further aspect, there is provided a method of expressing an expression product in the NS, the method comprising introducing into the NS cell an expression cassette comprising a synthetic NS-specific promoter comprising or consisting of SEQ ID NO: 13 operably linked to the expression product. Suitably, the expression product is expressed in excitatory neurones, oligodendrocytes and astrocytes.

In a further aspect, there is provided a method of expressing an expression product in the NS, the method comprising introducing into the NS cell an expression cassette comprising a synthetic NS-specific promoter comprising or consisting of SEQ ID NO: 14 operably linked to the expression product. Suitably, the expression product is expressed in excitatory neurones, oligodendrocytes and astrocytes.

In a further aspect, there is provided a method of expressing an expression product in the NS, the method comprising introducing into the NS cell an expression cassette comprising a synthetic NS-specific promoter comprising or consisting of SEQ ID NO: 33 operably linked to the expression product. Suitably, the expression product is expressed in neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 34 operably linked to the expression product. Suitably, the expression product is expressed in neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 35 operably linked to the expression product. Suitably, the expression product is expressed in neurones and astrocytes.

In a further aspect, there is provided a method of expressing an expression product in the NS, the method comprising introducing into the NS cell an expression cassette comprising a synthetic NS-specific promoter comprising or consisting of SEQ ID NO: 36 operably linked to the expression product. Suitably, the expression product is expressed in neurones and astrocytes.

### BRIEF DESCRIPTION OF THE FIGURES

FIG. 1A shows the expression pattern of the HTT gene in a sagittal section from an adult mouse brain (taken from the Allen Mouse brain atlas; mouse.brain-map.org). HTT (huntingtin) is highly expressed in throughout the brain.

FIG. 1B shows the expression pattern of the CYP46A1 gene in a coronal section from an adult mouse brain (taken from the Allen Mouse brain atlas; mouse.brain-map.org). CYP46A1 is widely expressed in the brain.

FIG. 2A shows the median GFP expression of synthetic NS-specific promoters SP0013, SP0014, SP0030, SP0031, SP0032, SP0019, SP0020, SP0021, SP0022, SP0011, SP0034, SP0035, SP0036 and control promoters Synapsin-1 relative to control promoter CAG in neuroblastoma-derived SH-SY5Y cells. NTC denotes non-transfected cells. The data is collected from three biological replicates, each of which is the average of two technical replicates. Error bars are standard error.

FIG. 2B shows the transfection efficiency in neuroblastoma-derived SH-SY5Y cells when transfected with synthetic NS-specific promoters SP0013, SP0014, SP0030, SP0031, SP0032, SP0019, SP0020, SP0021, SP0022, SP0011, SP0034, SP0035, SP0036 or control promoters Synapsin-1 and CAG, operably linked to GFP. NTC denotes non-transfected cells. The data is collected from three biological replicates, each of which is the average of two technical replicates. Error bars are standard error. GFP positive % denotes the % of all cells which were GFP positive.

### DETAILED DESCRIPTION OF EMBODIMENTS OF THE INVENTION AND EXAMPLES

### CREs and Functional Variants Thereof

Disclosed herein are various CREs that can be used in construction of NS-specific promoters. Suitably, the CREs are NS-specific, suitably CNS-specific and/or PNS-specific.

These CREs are generally derived from genomic promoter and enhancer sequences, but they are used herein in contexts quite different from their native genomic environment.

Generally, the CREs constitute small parts of much larger genomic regulatory domains, which control expression of the genes with which they are normally associated. It has been surprisingly found that these CREs, many of which are very small, can be isolated form their normal environment and retain NS-specific regulatory activity. This is surprising because the removal of a regulatory sequence from the complex and “three dimensional” natural context in the genome often results in a significant loss of activity, so there is no reason to expect a given CRE to retain the levels of activity observed once removed from their natural environment. It is even more surprising when a CRE retain NS-specific activity in an AAV vector. This is particularly the case as an AAV vector comprises Inverted Terminal Repeat (ITR) and has a different DNA structure compared to the genome and both ITRs and the DNA structure are known to influence the activity of CREs.

It should be noted that the sequences of the CREs of the present invention can be altered without causing a substantial loss of activity. Functional variants of the CREs can be prepared by modifying the sequence of the CREs, provided that modifications which are significantly detrimental to activity of the CRE are avoided. In view of the information provided in the present disclosure, modification of CREs to provide functional variants is straightforward. Moreover, the present disclosure provides methodologies for simply assessing the functionality of any given CRE variant.

The relatively small size of certain CREs according to the present invention is advantageous because it allows for the CREs, more specifically promoters containing them, to be provided in vectors while taking up the minimal amount of the payload of the vector. This is particularly important when a CRE is used in a vector with limited capacity, such as an AAV-based vector.

CREs of the present invention comprise certain NS-specific transcription factor binding sites (TFBS). It is generally desired that in functional variants of the CREs these NS-specific TFBS remain functional. The skilled person is well aware that TFBS sequences can vary yet retain functionality. In view of this, the sequence for a TFBS is typically illustrated by a consensus sequence from which some degree of variation is typically present. Further information about the variation that occurs in a TFBS can be illustrated using a positional weight matrix (PWM), which represents the frequency with which a given nucleotide is typically found at a given location in the consensus sequence. Details of TF consensus sequences and associated positional weight matrices can be found in, for example, the Jaspar or Transfac databases http://jaspar.genereg.net/ and http://gene-regulation.com/pub/databases.html). This information allows the skilled person to modify the sequence in any given TFBS of a CRE in a manner which retains, and in some cases even increases, CRE functionality. In view of this the skilled person has ample guidance on how the TFBS for any given TF can be modified, while maintaining ability to bind the desired TF; the Jaspar system will, for example, score a putative TFBS based on its similarity to a given PWM. Furthermore, CREs can be scanned against all PWM from JASPAR database to identify/analyse all TFBS. The skilled person can of course find additional guidance in the literature, and, moreover, routine experimentation can be used to confirm TF binding to a putative TFBS in any variant CRE. It will be apparent that significant sequence modification in a CRE, even within TFBS in a CRE, can be made while retaining function. CREs of the present invention can be used in combination with a wide range of suitable minimal promoters or NS-specific proximal promoters, suitably CNS-specific proximal promoters and/or PNS-specific proximal promoters.

Functional variants of a CRE include sequences which vary from the reference CRE element, but which substantially retain activity as NS-specific CREs. It will be appreciated by the skilled person that it is possible to vary the sequence of a CRE while retaining its ability to recruit suitable NS-specific transcription factors (TFs) and thereby enhance expression. A functional variant of a CRE can comprise substitutions, deletions and/or insertions compared to a reference CRE, provided they do not render the CRE substantially non-functional.

In some embodiments, a functional variant of a CRE can be viewed as a CRE which, when substituted in place of a reference CRE in a promoter, substantially retains its activity. For example, a NS-specific promoter which comprises a functional variant of a given CRE preferably retains at least 80% of its activity, more preferably at least 90% of its activity, more preferably at least 95% of its activity, and yet more preferably 100% of its activity (compared to the reference promoter comprising the unmodified CRE).

Suitably, functional variants of a CRE retain a significant level of sequence identity to a reference CRE. Suitably functional variants comprise a sequence that is at least 70% identical to the reference CRE, more preferably at least 80%, 90%, 95% or 99% identical to the reference CRE.

Retention of activity can be assessed by comparing expression of a suitable reporter under the control of the reference promoter with an otherwise identical promoter comprising the substituted CRE under equivalent conditions. Suitable assays for assessing NS-specific promoter activity are disclosed herein, e.g. in the examples.

In some embodiments, a CRE can be combined with one or more additional CREs to create a cis-regulatory module (CRM). Additional CREs can be provided upstream of the CREs according to the present invention, or downstream of the CRE according to the present invention. The one or more additional CREs can be CREs disclosed herein, or they can be other CREs known in the art. Suitably, when the one or more additional CREs are not CREs according to the present invention, the one or more additional CRE may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. Suitably, the additional CREs are NS-specific.

CREs according to the present invention or CRMs comprising CREs according to the present invention may comprise one or more additional regulatory elements. For example, they may comprise an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the CRE or CRM substantially non-functional.

A promoter comprising CREs according to the present invention may comprise spacers between the CRM and the minimal or proximal promoter and/or between CREs. Additionally, or alternatively, a spacer may be present on the 5′ end of the CRM.

It will be apparent that a CRE according to the present invention or a CRM comprising a CRE according to this invention, or functional variants thereof, can be combined with any suitable promoter elements in order to provide a synthetic NS-specific promoter according to the present invention. Suitably, the promoter element is a NS-specific proximal promoter.

In many instances, shorter promoter sequences are preferred, particularly for use in situations where a vector (e.g. a viral vector such as AAV) has limited capacity. Accordingly, in some embodiments the CREs according to the present invention or functional variants thereof have a length of 600 or fewer nucleotides, for example 600, 500, 450, 400, 350, 300, 250, 200, 150, 100, 75, 60, 50 or fewer nucleotides. Suitably, the synthetic NS specific CRM comprising at least one of the CREs according to SEQ ID NOs 19-24, 27, 28, 37, 38 or a functional variant thereof has length of 1000 or fewer nucleotides, for example 950, 900, 850, 800, 750, 700, 650, 600, 550, 500, 450, 400, 350, 300, 250, 200, 150, 100, 75, 60, 50 or fewer nucleotides.

### CRMs and Functional Variants Thereof

Various synthetic NS-specific CRMs are disclosed herein that can be used in the constructions of synthetic NS-specific promoters. CRMs of the present invention can be used in combination with a wide range of suitable minimal promoters or NS-specific proximal promoters.

Functional variants of a CRM include sequences which vary from the reference CRM element, but which substantially retain activity as NS-specific CRMs. It will be appreciated by the skilled person that it is possible to vary the sequence of a CRM while retaining its ability to recruit suitable NS-specific transcription factors (TFs) and thereby enhance expression. A functional variant of a CRM can comprise substitutions, deletions and/or insertions compared to a reference CRM, provided they do not render the CRM substantially non-functional.

In some embodiments, a functional variant of a CRM can be viewed as a CRM which, when substituted in place of a reference CRM in a promoter, substantially retains its activity. For example, a NS-specific promoter which comprises a functional variant of a given CRM preferably retains at least 80% of its activity, more preferably at least 90% of its activity, more preferably at least 95% of its activity, and yet more preferably 100% of its activity (compared to the reference promoter comprising the unmodified CRM).

Suitably, functional variants of a CRM retain a significant level of sequence identity to a reference CRM. Suitably functional variants comprise a sequence that is at least 70% identical to the reference CRM, more preferably at least 80%, 90%, 95% or 99% identical to the reference CRM.

Retention of activity can be assessed by comparing expression of a suitable reporter under the control of the reference promoter with an otherwise identical promoter comprising the substituted CRM under equivalent conditions. Suitable assays for assessing NS-specific promoter activity are disclosed herein, e.g. in the examples.

Functional variants of a given CRM can, in some embodiments, comprise functional variants of one or more of the CREs present in the reference CRM. For example, functional variants of a given CRM can comprise functional variants of 1 or 2 of the CREs present in the reference CRM.

Functional variants of a given CRM can, in some embodiments, comprise the same combination CREs as a reference CRM, but the CREs can be present in a different order from the reference CRM. It is usually preferred that the CREs are present in the same order as the reference CRM (thus, the functional variant of a CRM suitably comprises the same permutation of the CREs as set out in a reference CRM).

Functional variants of a given CRM can, in some embodiments, comprise one or more additional CREs to those present in a reference CRM. Additional CREs can be provided upstream of the CREs present in the reference CRM, downstream of the CREs present in the reference CRM, and/or between the CREs present in the reference CRM. The one or more additional CREs can be CREs disclosed herein, or they can be other CREs known in the art. Suitably, when the one or more additional CREs are not CREs according to the present invention, the one or more additional CRE may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. Generally, it is preferred that a functional variant of a given CRM comprises the same CREs (or functional variants thereof) and does not comprise additional CREs.

Functional variants of a given CRM can comprise one or more additional regulatory elements compared to a reference CRM. For example, they may comprise an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the CRM substantially non-functional.

Functional variants of a given CRM can comprise additional spacers between adjacent CREs or, if one or more spacer are present in the reference CRM, said one or more spacers can be longer or shorter than in the reference CRM. Spacers present in the reference CRM can be removed in the functional variant.

It will be apparent that the CRMs as disclosed herein, or functional variants thereof, can be combined with any suitable promoter elements in order to provide a synthetic NS-specific promoter according to the present invention. Suitably, the promoter element is a NS-specific proximal promoter.

In many instances, shorter promoter sequences are preferred, particularly for use in situations where a vector (e.g. a viral vector such as AAV) has limited capacity. Accordingly, in some embodiments the synthetic NS-specific CRM has length of 500 or fewer nucleotides, for example 450, 400, 350, 300, 250, 200, 150, 100, 75, 60, 50 or fewer nucleotides.

### Synthetic NS-Specific Promoters and Functional Variants Thereof

Various synthetic NS-specific promoters are disclosed herein. A functional variant of a reference synthetic NS-specific promoter is a promoter which comprises a sequence which varies from the reference synthetic NS-specific promoter, but which substantially retains NS-specific promoter activity. It will be appreciated by the skilled person that it is possible to vary the sequence of a synthetic NS-specific promoter while retaining its ability to recruit suitable NS-specific transcription factors (TFs) and to recruit RNA polymerase II to provide NS-specific expression of an operably linked sequence (e.g. an open reading frame). A functional variant of a synthetic NS-specific promoter can comprise substitutions, deletions and/or insertions compared to a reference promoter, provided such substitutions, deletions and/or insertions do not render the synthetic NS-specific promoter substantially non-functional compared to the reference promoter.

Accordingly, in some embodiments, a functional variant of a synthetic NS-specific promoter can be viewed as a variant which substantially retains the NS-specific promoter activity of the reference promoter. For example, a functional variant of a synthetic NS-specific promoter preferably retains at least 70% of the activity of the reference promoter, more preferably at least 80% of its activity, more preferably at least 90% of its activity, more preferably at least 95% of its activity, and yet more preferably 100% of its activity.

Functional variants of a synthetic NS-specific promoter often retain a significant level of sequence similarity to a reference synthetic NS-specific promoter. In some embodiments, functional variants comprise a sequence that is at least 70% identical to the reference synthetic NS-specific promoter, more preferably at least 80%, 90%, 95% or 99% identical to the reference synthetic NS-specific promoter.

Activity in a functional variant can be assessed by comparing expression of a suitable reporter under the control of the reference synthetic NS-specific promoter with the putative functional variant under equivalent conditions. Suitable assays for assessing NS-specific promoter activity are disclosed herein, e.g. in the examples.

Functional variants of a given synthetic NS-specific promoter can comprise functional variants of a CRE present in the reference synthetic NS-specific promoter. Functional variants of a given synthetic NS-specific promoter can comprise functional variants of the CRM present in the reference synthetic NS-specific promoter. Functional variants of a given synthetic NS-specific promoter can comprise functional variants of the promoter element, or a different promoter element when compared to the reference synthetic NS-specific promoter.

Functional variants of a given synthetic NS-specific promoter can comprise one or more additional CREs to those present in a reference synthetic NS-specific promoter. Additional CREs can, for example, be provided upstream of the CREs present in the reference synthetic NS-specific promoter or downstream of the CREs present in the reference synthetic NS-specific promoter. The one or more additional CREs can be CREs disclosed herein, or they can be other CREs known in the art. Suitably, when the one or more additional CREs are not CREs according to the present invention, the one or more additional CRE may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121.

Functional variants of a given synthetic NS-specific promoter can comprise additional spacers between adjacent elements (CREs, CRM or promoter element) or, if one or more spacers are present in the reference synthetic NS-specific promoter, said one or more spacers can be longer or shorter than in the reference synthetic NS-specific promoter. Alternatively, if one or more spacers are present in the reference synthetic NS-specific promoter, these spacers may be removed in the functional variant.

It will be apparent that synthetic NS-specific promoters of the present invention can comprise a CRE of the present invention or a CRM comprising a CRE of the present invention and additional regulatory sequences. For example, they may comprise one or more additional CREs, an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional.

Preferred synthetic NS-specific promoters of the present invention exhibit NS-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by the Synapsin-1, Camk2a, GFAP, MBP, IBA1 or the NSE promoter in NS cells. In many cases higher levels of promoter activity is preferred, but this is not always the case; thus, in some cases more moderate levels of expression may be preferred. In some cases, it is desirable to have available a range of promoters of different activity levels to allow the level of expression to be tailored to requirements; the present disclose provides promoters with such a range of activities. Activity of a given synthetic NS-specific promoter of the present invention compared to a known promoter can be assessed by comparing NS-specific expression of a reporter gene under control of the synthetic NS-specific promoter with expression of the same reporter under control of the known promoter, when the two promoters are provided in otherwise equivalent expression constructs and under equivalent conditions.

In addition to different activity levels, in some cases, it is desirable to have available a range of promoters with activity in different regions, such as different regions of the NS or PNS, suitably the CNS, suitably different regions of the brain or spinal cord. Additionally, it may be desirable to have a range of promoters with different activity levels across different regions to allow the level of expression to be tailored to requirements. In some cases, expression in a specific region is desired. In some embodiments, expression in the brain is desired with little or no expression in the rest of the CNS, the PNS or rest of the body. In some embodiments, expression in the spinal cord is desired with little or no expression in the rest of the CNS, the PNS or rest of the body. Suitably, expression may be required in multiple regions within the brain or spinal cord. In some preferred embodiments, the NS-specific promoter according to the present invention shows activity in any or all of the following brain regions: the striatum, the substantia nigra, layers 3, 5 and 6 of the cerebral cortex (the cerebral cortex includes the frontal lobe, pariental lobe, occipital lobe and temporal lobe), the hippocampus, the cerebellum, the hypothalamus and the thalamus. In some embodiments, the NS-specific promoter according to the present invention shows activity the brain areas mentioned above with little or no activity in other areas of the brain, other areas of the CNS, the PNS or other areas of the body.

In addition to different activity levels and different areas of activity, in some cases, it is desirable to have available a range of promoters with activity in different cells or combinations of cells, such as CNS cells and/or PNS cells. In some preferred embodiments, the NS-specific promoter according to the present invention shows activity in PNS and/or PNS neurones. In some preferred embodiments, the NS-specific promoter according to the present invention shows activity in neurones and astrocytes. In some preferred embodiments, the NS-specific promoter according to the present invention shows activity in neurones and oligodendrocytes. In some preferred embodiments, the NS-specific promoter according to the present invention shows activity in neurones, astrocytes and oligodendrocytes. In some embodiments, the NS-specific promoter according to the present invention shows activity in CNS neurones with little or no expression in other CNS or PNS cell types or CNS or PNS subtypes. In some embodiments, the NS-specific promoter according to the present invention shows activity in CNS and PNS neurones with little or no expression in other CNS or PNS cell types or CNS or PNS subtypes. In some embodiments, the NS-specific promoter according to the present invention shows activity in PNS neurones with little or no expression in other CNS or PNS cell types or CNS or PNS subtypes. In some embodiments, the NS-specific promoter according to the present invention shows activity in neurones and oligodendrocytes with little or no expression in other CNS or PNS cell types or CNS or PNS subtypes. In some embodiments, the NS-specific promoter according to the present invention shows activity in neurones and astrocytes with little or no expression in other CNS or PNS cell types or CNS or PNS subtypes. In some embodiments, the NS-specific promoter according to the present invention shows activity in neurones, oligodendrocytes and astrocytes with little or no expression in other CNS or PNS cell types or CNS or PNS subtypes. In some embodiments, the NS-specific promoter according to the present invention shows activity in neurones and satellite cells with little or no expression in other CNS or PNS cell types or CNS or PNS subtypes.

Alternatively, it might be preferred to have a widespread expression in all or almost all regions of the CNS and/or PNS. In some embodiments, it is desirable to have widespread expression in all or almost all regions of the CNS, suitably all areas of the brain. This may be the case, for example, in the treatment of diseases such as Huntington's disease where expression is desired throughout the CNS, suitably throughout the brain.

In one embodiment, the NS-specific promoter according to the invention shows activity in neurones, astrocytes and/or oligodendrocytes in almost all areas of the brain, suitably in the striatum, the substantia nigra, layers 3, 5 and 6 of the cerebral cortex (the cerebral cortex includes the frontal lobe, pariental lobe, occipital lobe and temporal lobe), the hippocampus, the cerebellum, the hypothalamus and the thalamus.

Preferred synthetic NS-specific promoters of the present invention exhibit neurone-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by Synapsin-1 in neurones. Activity of a given synthetic NS-specific promoter of the present invention compared to Synapsin-1 can be assessed by comparing neurone-specific expression of a reporter gene under control of the synthetic NS-specific promoter with expression of the same reporter under control of Synapsin-1 promoter in neurones, when the two promoters are provided in otherwise equivalent expression constructs and under equivalent conditions. In some embodiments a synthetic NS-specific promoter of the invention is able to increase expression of a gene (e.g. a therapeutic gene or gene of interest) in the neurones of a subject by at least 20%, at least 40%, at least 60%, at least 80%, at least 100%, at least 200%, at least 300%, at least 500%, at least 1000% or more relative to a known neurone-specific promoter, suitably Synapsin-1.

Preferred synthetic NS-specific promoters of the present invention exhibit astrocyte-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by GFAP promoter in astrocytes. Activity of a given synthetic NS-specific promoter of the present invention compared to GFAP can be assessed by comparing astrocyte-specific expression of a reporter gene under control of the synthetic NS-specific promoter with expression of the same reporter under control of GFAP promoter in astrocytes, when the two promoters are provided in otherwise equivalent expression constructs and under equivalent conditions. In some embodiments a synthetic NS-specific promoter of the invention is able to increase expression of a gene (e.g. a therapeutic gene or gene of interest) in the astrocytes of a subject by at least 20%, at least 40%, at least 60%, at least 80%, at least 100%, at least 200%, at least 300%, at least 500%, at least 1000% or more relative to a known astrocyte-specific promoter, suitably GFAP.

Preferred synthetic NS-specific promoters of the present invention exhibit oligodendrocyte-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by MBP promoter in oligodendrocytes. Activity of a given synthetic NS-specific promoter of the present invention compared to MBP can be assessed by comparing oligodendrocyte-specific expression of a reporter gene under control of the synthetic NS-specific promoter with expression of the same reporter under control of MBP promoter in astrocytes, when the two promoters are provided in otherwise equivalent expression constructs and under equivalent conditions. In some embodiments a synthetic NS-specific promoter of the invention is able to increase expression of a gene (e.g. a therapeutic gene or gene of interest) in the oligodendrocytes of a subject by at least 20%, at least 40%, at least 60%, at least 80%, at least 100%, at least 200%, at least 300%, at least 500%, at least 1000% or more relative to a known oligodendrocyte-specific promoter, suitably MBP.

In some embodiments a synthetic NS-specific promoter of the invention is able to increase expression of a gene (e.g. a therapeutic gene or gene of interest) in the CNS and/or PNS of a subject or in a CNS and/or PNS cell by at least 20%, at least 40%, at least 60%, at least 80%, at least 100%, at least 200%, at least 300%, at least 500%, at least 1000% or more relative to a known NS-specific promoter, suitably the Syn1, Camk2a or NSE promoter.

Preferred synthetic NS-specific promoters of the present invention exhibit activity in non-NS cells (e.g. Huh7 and HEK293 cells) which is 50% or less when compared to CMV-IE, preferably 25% or less than CMV-IE, more preferably 10% or less than CMV-IE, and in some cases 5% or less than CMV-IE, or 1% or less than CMV-IE.

In many instances, shorter promoter sequences are preferred, particularly for use in situations where a vector (e.g. a viral vector such as AAV) has limited capacity. Accordingly, in some embodiments the synthetic NS-specific promoter has length of 1000 or fewer nucleotides, for example, 900, 800, 700,600, 500, 450, 400, 350, 300, 250, 200, 150, 100, or fewer nucleotides.

Particularly preferred synthetic NS-specific promoters are those that are both short and which exhibit high levels of activity.

It is surprising when a NS-specific promoter retains NS-specific activity in an AAV vector as the AAV vector's ITRs and different DNA structure compared to the genome are known to influence the activity of promoters, often the ITRs and different DNA structure negatively impact the activity of promoters.

### Other CREs

The synthetic NS-specific promoter may comprise one or more additional CREs or functional variants thereof. The CREs may be CREs according to the present invention (any one of SEQ ID NOs: 19-24, 27, 28, 37, 38), functional variants thereof, or other CREs known in the art.

The CRE according to the present invention may be combined with one or more additional CREs to form a cis-regulatory module (CRM). Suitably, the one or more additional CREs may be CREs according to the present invention (any one of SEQ ID NOs 19-24, 27, 28, 37, 38), or functional variants thereof, or other CREs known in the art.

The CRM according to the present invention may comprise one or more additional CREs. Suitably, the one or more additional CREs may be CREs according to the present invention (any one of SEQ ID NOs 19-24, 27, 28, 37, 38), or functional variants thereof, or other CREs known in the art.

The minimal or proximal promoter according to the present invention can be operably linked one or more CREs or a CRM. The one or more CREs may CREs according to this invention (any one of SEQ ID NOs 19-24, 27, 28, 37, 38), or functional variants thereof, or other CREs known in the art. The CRM may comprise CREs according to this invention (any one of SEQ ID NOs 19-24, 27, 28, 37, 38), or functional variants thereof, or other CREs known in the art.

Preferably, when the one or more CREs are not CREs according to the present invention (herein called other CREs), the other CREs are NS-specific CREs. Suitably, when the one or more CREs are not CREs according to the present invention, the one or more CRE may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. CREs according to SEQ ID NOs: 1-8 from WO 2019/199867A1 are CREs specific for inhibitory GABAergic interneurons. CREs according to SEQ ID NOs: 1-7 from WO 2020/076614A1 are CREs specific for forebrain GABAergic interneurons. CREs according to SEQ ID NOs: 25-34, 36-51, 177-178, 188 from WO 2020/097121 are CREs specific for glutamatergic neurones. The CRE according to SEQ ID NO: 35 from WO 2020/097121 is a CRE specific for glutamatergic and GABAergic neurones.

### Synthetic NS-Specific Expression Cassettes

The present invention also provides a synthetic NS-specific expression cassette comprising a synthetic NS-specific promoter of the present invention operably linked to a sequence encoding an expression product, suitably a gene (e.g. a transgene).

Suitably therefore, the expression product may be a gene. Where the gene encodes a protein, it can be essentially any type of protein. By way of non-limiting example, the protein can be an enzyme, an antibody or antibody fragment (e.g. a monoclonal antibody), a viral protein (e.g. REP-CAP, REV, VSV-G, or RD114), a therapeutic protein, or a toxic protein (e.g. Caspase 3, 8 or 9).

In some preferred embodiments of the present invention, the gene encodes a therapeutic expression product, preferably a therapeutic polypeptide suitable for use in treating a disease or condition associated with aberrant gene expression, optionally in the NS, suitably in the CNS and/or the PNS.

In some embodiments, therapeutic expression products include those useful in the treatment of CNS and/or PNS diseases. The terms “CNS disease” and “PNS disease” are, in principle, understood by the skilled person. The term relates to a disease amenable to treatment and/or prevention by administration of an active compound to the CNS and/or PNS, in particular to a CNS and/or PNS cell. In some embodiments, the CNS and/or PNS disease is a neurological disease and/or disorder.

The disease may be a disease of the CNS and/or the PNS.

As a non-limiting example, the CNS disease may be selected from: Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diffuse Sclerosis, Dravet Syndrome, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease-Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Periventricular Leukomalacia, Persistent Vegetative State, Pervasive Developmental Disorders, Phytanic Acid Storage Disease, Pick's Disease, Pinched Nerve, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEl), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

As a non-limiting example, the PNS disease may be selected from: accessory nerve disorder, alcoholic polyneuropathy, anestesia dolorosa, anti-MAG peripheral neuropathy, autoimmune autonomic ganglionopathy, autonomic dysreflexia, autonomic neuropathy, axillary nerve dysfunction, axillary nerve palsy, Charcot-Marie-Tooth disease, chemotherapy-induced peripheral neuropathy, chronic solvent-induced encephalopathy, CMV polyradiculomyelopathy, congenital insensitivity to pain with anhidrosis, denervation, diabetic neuropathy, dysautonomia, erythromelalgia, facial nerve paralysis, familial dysautonomia, Guillain-Barre syndrome, hereditary sensory and autonomic neuropathy, Horner's syndrome, nerve compression syndrome, nerve injury, neurapraxia, neuritis, orthostatic hypotension, orthostatic intolerance, paroxysmal sympathetic hyperactivity, peripheral mononeuropathy, peripheral neuropathy, Piriformis syndrome, plexopathy, polyneuropathy, postural orthostatic tachycardia syndrome, primary autonomic failure, Pronator teres syndrome, proximal diabetic neuropathy, Pudendal nerve entrapment, pure autonomic failure, quadrilateral space syndrome, radial nerve dysfunction, radial neuropathy, radiation-induced lumbar plexopathy, radiculopathy, sciatica, small fiber peripheral neuropathy, thoracic outlet syndrome, Ulnar neuropathy, vasculitic neuropathy, Villaret's syndrome, Wartenberg's syndrome and Winged scapula.

Some disease may be diseases of the both the CNS and PNS.

In some embodiments, the CNS disease is selected from the list consisting of: dopamine transporter deficiency syndrome, an attention deficit/hyperactivity disorder (ADHD), bipolar disorder, epilepsy, multiple sclerosis, tauopathies, Alzheimer's disease, Huntington's disease, Parkinson's disease, Krabbe's disease, adrenoleukodystrophy, motor neurone disease, cerebral palsy, Batten disease, Gaucher disease, Tay Sachs disease, Rett syndrome, Sandhoff disease, Charcot-Marie-Tooth disease, Angelman syndrome, Canavan disease, Late infantile neuronal ceroid lipofuscinosis, Mucopolysaccharidosis IIIA, Mucopolysaccharidosis IIIB, Metachromatic leukodystrophy, heritable lysosomal storage diseases such as Niemann-Pick disease type C1, and/or neuronal ceroid lipofuscinoses such as Batten disease, progressive supranuclear palsy, corticobasal syndrome, and brain cancer (including astrocytomas and glioblastomas).

In one embodiment, the CNS disease is Huntington's disease.

Various expression products suitable for treating the above conditions have been described in the art. Suitably, the sequence encoding an expression product operably linked to the synthetic NS-specific promoter according to the invention may be one of the genes selected from the group consisting of: NPC1, EAAT2, NPY, CYP46A1, GLB1, APOE (e.g. ApoE2, ApoE3 or ApoE4), HEX, CLN1, CLN2, CLN3, CLN4, CLN5, CLN6, SUMF1, DCTN1, PRPH, SOD1, NEFH, GBA, IDUA, NAGLU, GUSB, ARSA, MANB, AADC, GDNF, NTN, ASP, MECP2, PTCHD1, GJB1, UBE3A, HEXA, MOG. Additionally, or alternatively, expression product operably linked to the synthetic NS-specific promoter according to the invention may be a miRNA/CRISPR Cas9 directed to the disease allele. Suitably, the sequence encoding an expression product operably linked to the synthetic NS-specific promoter according to the invention may be the gene CYP46A1.

CYP46A1 is the rate-limiting enzyme for cholesterol degradation and it has been found to play a beneficial role in multiple CNS diseases. CYP46A1 inhibition may contribute to inducing and/or aggravating Alzheimer's disease via increased amount of viral cholesterol, as described in (Djelti et al., 2015) which is incorporated herein by reference. CYP46A1 has also been found to be neuroprotective in Huntington's disease, as described in (Boussicault et al., 2016) which is incorporated herein by reference. Therefore, the CYP46A1 gene is a particularly preferred sequence encoding an expression product. In some preferred embodiments, the CYP46A1 gene is operably linked to the synthetic NS-specific promoter according to the invention. Suitably, the CYP46A1 gene is operably linked to a synthetic promoter which is active in all areas of the CNS (pan-CNS) or a promoter which is active in more than 5, 6, 7, 8 or 9 of the areas of the brain recited above. Expression of CYP46A1 in all areas of the CNS or more than 5, 6, 7, 8 or 9 of the areas of the brain recited above may be beneficial as CYP46A1 expression by the ubiquitous promoters CMV or CAG was found to be beneficial in a mouse Huntington's disease model (Kacher et al., 2019). Suitably, the CYP46A1 gene is operably linked to a synthetic promoter consisting or comprising of any one of SEQ ID NO: 1-14, 33-36.

In some embodiments, useful expression products include dystrophins (including micro-dystrophins), beta 1,4-n-acetylgalactosamine galactosyltransferase (GALGT2), carbamoyl synthetase I, alpha-1 antitrypsin, ornithine transcarbamylase, arginosuccinate synthetase, arginosuccinate lyase, arginase, fumarylacetacetate hydrolase, phenylalanine hydroxylase, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, and a cystic fibrosis transmembrane regulator (CFTR).

Still other useful expression products include enzymes useful in enzyme replacement therapy, and which are useful in a variety of conditions resulting from deficient activity of enzyme. For example, enzymes containing mannose-6-phosphate may be utilized in therapies for lysosomal storage diseases (e.g., a suitable gene includes that encoding β-glucuronidase (GUSB)).

In some embodiments, exemplary polypeptide expression products include neuroprotective polypeptides and anti-angiogenic polypeptides. Suitable polypeptides include, but are not limited to, glial derived neurotrophic factor (GDNF), fibroblast growth factor 2 (FGF-2), nurturin, ciliary neurotrophic factor (CNTF), nerve growth factor (NGF; e.g., nerve growth factor-.beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Fit-1, angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic expression products include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGFa., activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, useful expression products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful expression product include any one of the receptors for the hormones, growth factors, cytokines, lymphokines, regulatory proteins and immune system proteins. Useful heterologous nucleic acid sequences also include receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, useful expression products include non-naturally occurring polypeptides, such as chimeric or hybrid polypeptides having a non-naturally occurring amino acid sequence containing insertions, deletions or amino acid substitutions.

Further suitable expression products include micro RNA (miRNA), interfering RNA, antisense RNA, ribozymes, and aptamers.

In some embodiments of the invention, the synthetic NS-specific expression cassette comprises a gene useful for gene editing, e.g. a gene encoding a site-specific nuclease, such as a meganuclease, zinc finger nuclease (ZFN), transcription activator-like effector-based nuclease (TALEN), or the clustered regularly interspaced short palindromic repeats system (CRISPR-Cas). Suitably the site-specific nuclease is adapted to edit a desired target genomic locus by making a cut (typically a site-specific double-strand break) which is then repaired via non-homologous end-joining (NHEJ) or homology dependent repair (HDR), resulting in a desired edit. The edit can be the partial or complete repair of a gene that is dysfunctional, or the knock-down or knock-out of a functional gene. Alternatively, the edit can be via base editing or prime editing, using suitable systems which are known in the art. Suitably, for the treatment of Huntington's disease, the expression product may suppress the faulty huntingtin gene in the cells of a patient and/or can counteract or alleviate the detrimental effects of the faulty huntingtin gene in the cells of a patient.

Suitably the synthetic NS-specific expression cassette comprises sequences providing or coding for one or more of, and preferably all of, a ribosomal binding site, a start codon, a stop codon, and a transcription termination sequence. Suitably the expression cassette comprises a nucleic acid encoding a posttranscriptional regulatory element. Suitably the expression cassette comprises a nucleic acid encoding a polyA element.

### Vectors and Viral Particles

The present invention further provides a vector comprising a synthetic NS-specific promoter, or expression cassette according to the present invention.

In some embodiments of the invention, the vector is a plasmid. Such a plasmid may include a variety of other functional nucleic acid sequences, such as one or more selectable markers, one or more origins of replication, multiple cloning sites and the like. In some embodiments of the invention, the vector is a viral vector.

In some embodiments of the invention, the vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. For mammalian cells adenoviral vectors, the pSV and the pCMV series of vectors are particularly well-known non-limiting examples.

There are many well-known yeast expression vectors including, without limitation, yeast integrative plasmids (YIp) and yeast replicative plasmids (YRp). For plants the Ti plasmid of agrobacterium is an exemplary expression vector, and plant viruses also provide suitable expression vectors, e.g. tobacco mosaic virus (TMV), potato virus X, and cowpea mosaic virus.

In some preferred embodiments, the vector is a gene therapy vector. Various gene therapy vectors are known in the art, and mention can be made of AAV vectors, adenoviral vectors, retroviral vectors and lentiviral vectors. Where the vector is a gene therapy vector the vector preferably comprises a nucleic acid sequence operably linked to the synthetic NS-specific promoter of the invention that encodes a therapeutic product, suitably a therapeutic protein. The therapeutic protein may be a secretable protein. Non-limiting examples of secretable proteins are discussed above, and exemplary secretable therapeutic proteins, include clotting factors, such as factor VIII or factor IX, insulin, erythropoietin, lipoprotein lipase, antibodies or nanobodies, growth factors, cytokines, chemokines, plasma factors, toxic proteins, etc.

In some embodiments of the invention, the vector is a viral vector, such as a retroviral, lentiviral, adenoviral, herpes simplex or adeno-associated viral (AAV) vector. In some preferred embodiments, the vector is a lentiviral vector, suitably a lentiviral vector based on HIV-1. In some preferred embodiments the vector is an AAV vector. In some preferred embodiments the AAV has a serotype suitable or specifically optimised for CNS transduction. In order to transduce the cells of the PNS, it is not necessary to overcome the blood brain barrier so transduction of the cells of the PNS may be done by any suitable AAV serotype. In some embodiments, the AAV is selected from the group consisting of: AAV1, AAV2, AAV4, AAV5, AAV8, AAV9, AAVrh10, AAVDJ8, AAV2i8, AAV2.5, AAV2i8G9 and AAV2g9, or derivatives thereof.

AAV vectors are preferably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5. AAV9 is known to effectively transduce CNS cells and tissue particularly effectively, and thus AAV9 and derivatives thereof are of particular interest for targeting CNS cells and tissue. AAV2g9 is known to effectively transduce CNS cells and tissue particularly effectively, and thus AAV2g9 and derivatives thereof are of particular interest for targeting CNS cells and tissue. AAVrh10 is known to effectively transduce CNS cells and tissue particularly effectively, and thus AAVrh10 and derivatives thereof are of particular interest for targeting CNS cells and tissue. AAVrh10 is particularly preferred as systemic or intravenous delivery of AAVrh10 has been found to provide high transgene expression in the central nervous system as described in (Tanguy et al., 2015) which is incorporated herein by reference. AAVDJ8 is known to effectively transduce CNS cells and tissue particularly effectively, and thus AAVDJ8 and derivatives thereof are of particular interest for targeting CNS cells and tissue. AAVDJ8 is preferred as it has been shown to effectively target multiple regions of the brain and to effectively target astrocytes as described in (Hammond et al., 2017) which is incorporated herein by reference. AAV1, AAV2, AAV4, AAV5, AAV2i8, AAV2.5, AAV2i8G9 and AAV8 are also known to target CNS cells and tissue, and thus these AAV serotypes and derivates thereof are also of particular interest for targeting CNS cells and tissue.

The invention further provides recombinant virions (viral particles) comprising a vector as described above.

### Pharmaceutical Compositions

The vectors or virions of the present invention may be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit. Pharmaceutical compositions and delivery systems appropriate for the AAV vectors and methods and uses thereof are known in the art.

Accordingly, a further aspect of the invention provides a pharmaceutical composition comprising a vector or virion as described herein.

Relative amounts of the active ingredient (e.g. AAV vector particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient. By way of example, the composition may comprise between 0.1 percent and 100 percent, e.g., between 0.5 and 50 percent, between 1-30 percent, between 5-80 percent, at least 80 percent (w/w) active ingredient.

The pharmaceutical compositions can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the invention. In some embodiments, a pharmaceutically acceptable excipient may be at least 95 percent, at least 96 percent, at least 97 percent, at least 98 percent, at least 99 percent, or 100 percent pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Therapeutic and Other Methods and Uses

The present invention also provides a synthetic NS-specific promoter, expression cassette, vector, virion or pharmaceutical composition according to various aspects of the present invention for use in the treatment of a disease, preferably a disease associated with aberrant gene expression, optionally in the NS (e.g. a genetic NS disease). Relevant conditions, diseases and therapeutic expression products are discussed above.

The present invention also provides a synthetic NS-specific promoter, expression cassette, vector, virion according to the various aspects of the present invention for use as medicament.

The present invention also provides a synthetic NS-specific promoter, expression cassette, vector, virion according to the various aspects of the present invention for use the manufacture of a pharmaceutical composition for treatment of any condition or disease mentioned herein.

The present invention further provides a cell comprising a synthetic NS-specific promoter, expression cassette, vector, virion according to the various aspects of the invention. Suitably the cell is a eukaryotic cell. The eukaryotic cell can suitably be an animal (metazoan) cell (e.g. a mammalian cell). Suitably, the cell is a human cell.

In some embodiments of the invention, the cell is ex vivo, e.g. in cell culture. In other embodiments of the invention the cell may be part of a tissue or multicellular organism.

In a preferred embodiment, the cell is a NS cell, which may be ex vivo or in vivo. The NS cell may be a CNS cell or a PNS cell. The PNS cell may be a primary neurone, a Schwann cell or a satellite cell. The CNS cell may be a primary neurone, astrocyte, oligodendrocyte, microglial cell or an ependymal cell. Alternatively, the NS cell may be a NS-derived cell line, e.g. immortalised cell line. In one embodiment, the CNS cell is a neurone, astrocyte, or oligodendrocyte.

The cell may be present within a NS tissue environment (e.g. within the NS of an animal) or may be isolated from NS tissue, e.g. it may be in cell culture. Suitably the primary cell or the cell line is a human cell.

The synthetic NS-specific promoter, expression cassette, or vector, according to the invention may be inserted into the genome of the cell, or it may be episomal (e.g. present in an episomal vector).

In a further aspect the present invention provides a method for producing an expression product, the method comprising providing a synthetic NS-specific expression cassette according to the present invention (preferably in a vector as set out above) in a cell, preferably a NS cell, and expressing the gene present in the synthetic NS-specific expression cassette. The method suitably comprises maintaining said NS cell under suitable conditions for expression of the gene. In culture this may comprise incubating the cell, or tissue comprising the cell, under suitable culture conditions. The expression may of course be in vivo, e.g. in one or more cells in the NS of a subject.

Suitably the method comprises the step of introducing the synthetic NS-specific expression cassette into the NS cell. A wide range of methods of transfecting NS cells are well-known in the art. A preferred method of transfecting NS cells is transducing the cells with a viral vector comprising the synthetic NS-specific expression cassette, e.g. an AAV vector.

It will be evident to the skilled person that a synthetic NS-specific promoter, expression cassette, vector or virion according to various aspects of the invention may be used for gene therapy. Accordingly, the use of the such nucleic acid constructs in gene therapy forms part of the present invention.

The invention thus provides, in some embodiments, an expression cassette, vector or virion according to the present invention for use in gene therapy in a subject, preferably gene therapy through NS-specific expression of a therapeutic gene. The therapy may involve treatment of a disease through secretion of a therapeutic product from NS cells, suitably a disease involving aberrant gene expression in the NS, as discussed above.

The present invention also provides a method of expressing a therapeutic transgene in a NS cell, the method comprising introducing into the NS cell an expression cassette or vector according to the present invention. The NS cell can be in vivo or ex vivo.

The present invention also provides a method of gene therapy of a subject, preferably a human, in need thereof, the method comprising:
administering to the subject (suitably introducing into the NS of the subject) a synthetic NS-specific expression cassette, vector, virion or pharmaceutical composition of the present invention, which comprises a gene encoding a therapeutic product.

The method suitably comprises expressing a therapeutic amount of the therapeutic product from the gene in the NS of said subject. Various conditions and diseases that can be treated are discussed above. Genes encoding suitable therapeutic products are discussed above.

The method suitably comprises administering a vector or virion according to the present invention to the subject. Suitably the vector is a viral gene therapy vector, for example an AAV vector.

In some embodiments, the method comprises administering the gene therapy vector systemically. Systemic administration may be enteral (e.g. oral, sublingual, and rectal) or parenteral (e.g. injection). Preferred routes of injection include intravenous, intramuscular, subcutaneous, intra-arterial, intra-articular, intrathecal, and intradermal injections. In one embodiment, the gene therapy vector may be delivered by injection into the CSF pathway. Non-limiting examples of delivery to the CSF pathway include intrathecal (intraspinal injection, lumbar injection, or injection into the subarachnoid space) and intracerebroventricular administration. In some embodiments, the gene therapy vector may be delivered by intranasal delivery.

Particularly preferred route of administration of AAV vector or virion comprising the synthetic NS-specific promoter or expression cassette according to this invention is intravascular. Suitably, the AAV vector or virion comprising the synthetic NS-specific promoter or expression cassette according to this invention may be administered in the veins of the dorsal hand or the veins of the anterior forearm. Suitable veins in the anterior forearm are the cephalic, median or basilic veins. This is because this administration route is generally safe for the patient while still allowing some penetration past the blood brain barrier (into the CNS).

In some embodiments, the viral gene therapy vector may be administered concurrently or sequentially with one or more additional therapeutic agents or with one or more saturating agents designed to prevent clearance of the vectors by the reticular endothelial system.

Where the vector is an AAV vector, the dosage of the vector may be from 1×10¹⁰ gc/kg to 1×10¹⁵ gc/kg or more, suitably from 1×10¹² gc/kg to 1×10¹⁴ gc/kg, suitably from 5×10¹² gc/kg to 5×10¹³ gc/kg.

In general, the subject in need thereof will be a mammal, and preferably a primate, more preferably a human. Typically, the subject in need thereof will display symptoms characteristic of a disease. The method typically comprises ameliorating the symptoms displayed by the subject in need thereof, by expressing the therapeutic amount of the therapeutic product. In one embodiment, the therapeutic methods of the present invention may be used to reduce the decline of functional capacity and activities of daily living as measured by a standard evaluation system such as, but not limited to, the total functional capacity (TFC) scale. In one embodiment, the methods of the present invention may be used to improve performance on any assessment used to measure symptoms of neurological disease. Such assessments include, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

Gene therapy protocols for therapeutic gene expression in target cells in vitro and in vivo, are well-known in the art and will not be discussed in detail here. Briefly, they include intravenous or intraarterial administration (e.g. intra-corotid artery, intra-hepatic artery, intra-hepatic vein), intracerebroventricular, intracranial administration, intramuscular injection, interstitial injection, instillation in airways, application to endothelium and intra-hepatic parenchyme, of plasmid DNA vectors (naked or in liposomes) or viral vectors. Various devices have been developed for enhancing the availability of DNA to the target cell. While a simple approach is to contact the target cell physically with catheters or implantable materials containing the relevant vector, more complex approaches can use jet injection devices an suchlike. Gene transfer into mammalian NS cells can been performed using both ex vivo and in vivo procedures. The ex vivo approach typically requires harvesting of the NS cells, in vitro transduction with suitable expression vectors, followed by reintroduction of the transduced NS cells into the NS. This approach is generally less preferred due to the difficulty and danger of harvesting and reintroducing NS cells in the NS. In vivo gene transfer has been achieved by injecting DNA or viral vectors directly into the NS, e.g. by intracranial injection, or by intravenous or intraarterial injection of viral vectors.

In one embodiment, the gene therapy vector may be administered to a subject (e.g., to the NS of a subject) in a therapeutically effective amount to reduce the symptoms of neurological disease of a subject (e.g., determined using a known evaluation method). In some embodiments, the gene therapy vector and compositions comprising the gene therapy vector may be administered in a way which allows them to cross the blood-brain barrier, vascular barrier, or other epithelial barrier.

The gene therapy vectors may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. In some embodiments, the delivery of one treatment (e.g., gene therapy vectors) is still occurring when the delivery of the second (e.g., one or more therapeutic) begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The composition described herein and the at least one additional therapy can be administered simultaneously, in the same or in separate compositions, or sequentially. For sequential administration, the gene therapy vectors described herein can be administered first, and the one or more therapeutic can be administered second, or the order of administration can be reversed. The gene therapy vectors and the one or more therapeutic can be administered during periods of active disorder, or during a period of remission or less active disease. The gene therapy vectors can be administered before another treatment, concurrently with the treatment, post-treatment, or during remission of the disorder.

When administered in combination, the gene therapy vectors and the one or more therapeutic (e.g., second or third therapeutic), or all, can be administered in an amount or dose that is higher, lower or the same as the amount or dosage of each used individually, e.g., as a monotherapy. In certain embodiments, the administered amount or dosage of the gene therapy vectors and the one or more therapeutic (e.g., second or third agent), or all, is lower (e.g., at least 20%, at least 30%, at least 40%, or at least 50%) than the amount or dosage of each used individually. In other embodiments, the amount or dosage of the gene therapy vectors and the one or more therapeutic (e.g., second or third agent), or all, that results in a desired effect (e.g., treatment of a NS disease or disorder) is lower (e.g., at least 20%, at least 30%, at least 40%, or at least 50% lower) than the amount or dosage of each individually required to achieve the same therapeutic effect.

Compounds which may be used in combination with the AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3 (lithium) or PP2A, immunization with amyloid beta peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetyl-choline release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or Zolpidem for altered sleep-wake cycle), anticonvulsants (e.g., sodium valproate and carbamazepine for mania or hypomania) and mood stabilizers (e.g., lithium for mania or hypomania).

According to some preferred embodiments, the methods set out above may be used for the treatment of a subject with a NS-related disease as discussed above, suitably a CNS-disease such as Huntington's disease.

### Definitions and General Points

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds. -in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

The term “nervous system” or “NS” is well understood by the skilled person. The NS consists of the peripheral nervous system (PNS) and the central nervous system (CNS).

The term “central nervous system” or “CNS” is well understood by the skilled person. The CNS consists of the brain and the spinal cord. Preferably, the synthetic NS-specific promoters are active in the brain. The promoters of the present invention can be active in brain and/or spinal cord. Preferably, the CNS is a CNS of a mammal, even more preferably of a human subject.

The term “peripheral nervous system” or “PNS” is well understood by the skilled person. The PNS refers to parts of the nervous system outside the brain and spinal cord. The promoters of the present invention can be active in the PNS. Preferably, the PNS is a PNS of a mammal, even more preferably of a human subject.

The term “CNS cell” or “CNS cells” relates to cells which are found in CNS (CNS tissue) or which are derived from CNS tissue. CNS cells can be primary cells or a cell line (such as SH-Sy5y, Neuro2A, U87-MG). The CNS cells can be in in vivo (e.g. in CNS tissue) or in vitro (e.g. in cell culture). CNS cells comprise of neurones, astrocytes, oligodendrocytes, microglial cells and ependymal cells. Neurones as found in the CNS tissue comprise a cell body, a long axon and a synaptic terminal. A neurone transmits electric signals received in the cell body via its long axon to other cells close to their synaptic terminal.

Oligodendrocytes are a type of glial cell in the CNS which produces myelin sheaths which wrap around neuronal axon for faster electrical signal conduction. Astrocytes are star-shaped and are the most abundant cell type in the CNS. They have multiple roles which aid and regulate transmission of electrical impulses within the CNS and neuronal function. Microglia are the resident macrophage cell in the CNS and are involved in immune defence. Ependymal cells form the epithelial lining of the ventricles. The term “CNS cell” or “CNS cells” as used herein includes neurones, astrocytes, oligodendrocytes, microglial cells and/or ependymal cells. The promoters of the present invention can be active in any of the CNS cell (e.g. neurones). The promoters of the present invention may be active in two types of CNS cell (e.g. neurones and astrocytes). The promoters of the present invention may be active in three or more types of CNS cell (e.g. neurones, oligodendrocyte and astrocytes). The promoters of the present invention may be active in all types of CNS cells (neurones, astrocytes, oligodendrocytes, microglial cells and ependymal cells). Additionally, synthetic CNS-specific promoters of the present invention may be active in a subtype of a type of CNS cell such as dopaminergic neurones, GABAergic neurones or mature oligodendrocytes. In some embodiments, the synthetic CNS-specific promoters of the present invention may only be active in the subtype of a type of CNS cell such as dopaminergic neurones or mature oligodendrocytes. The CREs, proximal/minimal promoters and promoters of the present invention may be active in specific areas of the CNS, in specific CNS cells and/or CNS cell subtypes or both. In some embodiments, the CREs, proximal/minimal promoters and promoters of the present invention may be active in a specific CNS cell type, such as neurones, within all areas of the CNS. In other embodiments, the CREs, proximal/minimal promoters and promoters of the present invention may be active in a specific CNS cell type, such as neurones, within no more than one area of the CNS. In some embodiments, the CREs, proximal/minimal promoters and promoters of the present invention may be active in all CNS cells in all areas of the CNS. In some embodiments, the CREs, proximal/minimal promoters and promoters of the present invention may be active in all CNS cells in no more than one area of the CNS. In some embodiments, the CREs, proximal/minimal promoters and promoters of the present invention may be active in multiple CNS cells in multiple areas of the CNS.

The term “PNS cell” or “PNS cells” relates to cells which are found in PNS (PNS tissue) or which are derived from PNS tissue. PNS cells can be primary cells or a cell line. The PNS cells can be in in vivo (e.g. in PNS tissue) or in vitro (e.g. in cell culture). PNS cells comprise of neurones, Swann cells and satellite cells. Neurones as found in the PNS tissue comprise a cell body, a long axon and a synaptic terminal. A neurone transmits electric signals received in the cell body via its long axon to other cells close to their synaptic terminal. Schwann cells are a type of glial cell in the PNS which produces myelin sheaths which wrap around neuronal axon for faster electrical signal conduction. Satellite cells (satellite glial cells) are glial cells which cover the surface of neuron cell bodies in ganglia of peripheral nervous system and support neuronal cells. They have a variety of roles including controlling the microenvironment of sympathetic ganglia. The term “PNS cell” or “PNS cells” as used herein includes neurones, Swann cells and/or satellite cells. The promoters of the present invention can be active in any of the PNS cell (e.g. neurones). The promoters of the present invention may be active in two types of PNS cell (e.g. neurones and satellite cells). The promoters of the present invention may be active in all types of PNS cell (e.g. neurones, Swann cells and satellite cells). Additionally, synthetic NS-specific promoters of the present invention may be active in a subtype of a type of PNS cell such as sensory neurones. In some embodiments, the synthetic NS-specific promoters of the present invention may only be active in the subtype of a type of PNS cell such as sensory neurones. The CREs, proximal/minimal promoters and promoters of the present invention may be active in specific areas of the PNS, in specific PNS cells and/or PNS cell subtypes or both. In some embodiments, the CREs, proximal/minimal promoters and promoters of the present invention may be active in a specific PNS cell type, such as neurones, within all areas of the PNS. In other embodiments, the CREs, proximal/minimal promoters and promoters of the present invention may be active in a specific PNS cell type, such as neurones, within no more than one area of the PNS. In some embodiments, the CREs, proximal/minimal promoters and promoters of the present invention may be active in all PNS cells in all areas of the PNS. In some embodiments, the CREs, proximal/minimal promoters and promoters of the present invention may be active in all PNS cells in no more than one area of the PNS. In some embodiments, the CREs, proximal/minimal promoters and promoters of the present invention may be active in multiple PNS cells in multiple areas of the PNS.

The term “cis-regulatory element” or “CRE”, is a term well-known to the skilled person, and means a nucleic acid sequence such as an enhancer, promoter, insulator, or silencer, that can regulate or modulate the transcription of a neighbouring gene (i.e. in cis). CREs are found in the vicinity of the genes that they regulate. CREs typically regulate gene transcription by binding to TFs, i.e. they include transcription factor binding sites (TFBS). A single TF may bind to many CREs, and hence control the expression of many genes (pleiotropy). CREs are usually, but not always, located upstream of the transcription start site (TSS) of the gene that they regulate. “Enhancers” in the present context are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, the CREs of the present invention are NS-specific enhancer elements (often referred to as NS-specific CREs, or NS-specific CRE enhancers, or suchlike). In the present context, it is preferred that the CRE is located 2500 nucleotides or less from the transcription start site (TSS), more preferably 2000 nucleotides or less from the TSS, more preferably 1500 nucleotides or less from the TSS, and suitably 1000, 750, 500, 250, 200, 150, or 100 nucleotides or less from the TSS. CREs of the present invention are preferably comparatively short in length, preferably 1000 nucleotides or less in length, for example they may be 800, 700, 600, 500, 400, 300, 200, 175, 150, 90, 80, 70, 60 or 50 nucleotides or less in length. The CREs of the present invention are typically provided in combination with an operably linked promoter element, which can be a minimal promoter or proximal promoter; the CREs of the present invention may enhance NS-specific activity of the promoter element.

The term “cis-regulatory module” or “CRM” means a functional regulatory nucleic acid module, which usually comprises two or more CREs; in the present invention the CREs are typically NS-specific enhancers and thus the CRM is a synthetic NS-specific regulatory nucleic acid. A CRM may comprise a plurality of NS-specific CREs. Suitably, at least one of the CREs comprised in the CRM is a CRE according to SEQ ID NO: 19-24, 27, 28, 37, 38 or a functional variant thereof. Typically, the multiple CREs within the CRM act together (e.g. additively or synergistically) to enhance the transcription of a gene that a promoter comprising the CRM is operably associated with. There is considerable scope to shuffle (i.e. reorder), invert (i.e. reverse orientation), and alter spacing of CREs within a CRM. Accordingly, functional variants of CRMs of the present invention include, interalia, variants of the referenced CRMs wherein CREs within them have been shuffled and/or inverted, and/or the spacing between CREs has been altered.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. Many diverse promoters are known in the art.

The term “synthetic promoter” as used herein relates to a promoter that does not occur in nature. In the present context it typically comprises a CRE and/or CRM of the present invention operably linked to a minimal (or core) promoter or NS-specific proximal promoter (promoter element). The CREs and/or CRMs of the present invention serve to enhance NS-specific transcription of a gene operably linked to the synthetic promoter. Parts of the synthetic promoter may be naturally occurring (e.g. the minimal promoter or one or more CREs in the promoter), but the synthetic promoter as an entity is not naturally occurring. Alternatively, the synthetic promoter may be a shorter, truncated version of a promoter which occurs in nature.

As used herein, “minimal promoter” (also known as the “core promoter”) refers to a typically short DNA segment which is inactive or largely inactive by itself, but can mediate transcription when combined with other transcription regulatory elements. Minimal promoter sequences can be derived from various different sources, including prokaryotic and eukaryotic genes. Examples of minimal promoters include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box). A minimal promoter may also include some elements downstream of the TSS, but these typically have little functionality absent additional regulatory elements.

As used herein, “proximal promoter” relates to the minimal promoter plus at least some additional regulatory sequence, typically the proximal sequence upstream of the gene that tends to contain primary regulatory elements. It often extends approximately 250 base pairs upstream of the TSS, and includes specific TFBS. A proximal promoter may also include one or more regulatory elements downstream of the TSS, for example a UTR or an intron. In the present case, the proximal promoter may suitably be a shorter, truncated version of naturally occurring NS-specific proximal promoter. The proximal promoters of the present invention may be combined with one or more CREs or CRMs of the present invention. However, the proximal promoter can also be synthetic.

As used herein, “promoter element” refers to either a minimal promoter or proximal promoter as defined above. In the context of the present invention a promoter element may be combined with one or more CREs in order to provide a synthetic NS-specific promoter of the present invention.

A “functional variant” of a CRE, CRM, promoter element, promoter or other regulatory nucleic acid in the context of the present invention is a variant of a reference sequence that retains the ability to function in the same way as the reference sequence, e.g. as a NS-specific CRE, NS-specific CRM or NS-specific promoter. Alternative terms for such functional variants include “biological equivalents” or “equivalents”.

It will be appreciated that the ability of a given CRE, CRM, promoter or other regulatory sequence to function as a NS-specific enhancer is determined significantly by the ability of the sequence to bind the same NS-specific TFs that bind to the reference sequence. Accordingly, in most cases, a functional variant of a CRE or CRM will contain TFBS for the most or all of same TFs as the reference CRE, CRM or promoter. It is preferred, but not essential, that the TFBS of a functional variant are in the same relative positions (i.e. order and general position) as the reference CRE, CRM or promoter. It is also preferred, but not essential, that the TFBS of a functional variant are in the same orientation as the reference sequence (it will be noted that TFBS can in some cases be present in reverse orientation, e.g. as the reverse complement vis-à-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, the same position, in the same orientation and on the same strand as the reference sequence. It will also be appreciated that the sequences lying between TFBS (referred to in some cases as spacer sequences, or suchlike) are of less consequence to the function of the CRE or CRM. Such sequences can typically be varied considerably, and their lengths can be altered. However, in preferred embodiments the spacing (i.e. the distance between adjacent TFBS) is substantially the same (e.g. it does not vary by more than 20%, preferably by not more than 10%, and more preferably it is approximately the same) in a functional variant as it is in the reference sequence. It will be apparent that in some cases a functional variant of a CRE can be present in the reverse orientation, e.g. it can be the reverse complement of a CRE as described above, or a variant thereof.

Levels of sequence identity between a functional variant and the reference sequence can also be an indicator or retained functionality. High levels of sequence identity in the TFBS of the CRE, CRM or promoter is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

“NS-specific” or “NS-specific expression” refers to the ability of a cis-regulatory element, cis-regulatory module or promoter to enhance or drive expression of a gene in NS cells (or in NS-derived cells) in a preferential or predominant manner as compared to other tissues (e.g. liver, kidney, spleen, heart, muscle and lung). Expression of the gene can be in the form of mRNA or protein. In preferred embodiments, NS-specific expression is such that there is negligible expression in other (i.e. non-NS) tissues or cells, i.e. expression is highly NS-specific. NS-specific expression may be CNS-specific and/or PNS-specific expression.

“CNS-specific” or “CNS-specific expression” refers to the ability of a cis-regulatory element, cis-regulatory module or promoter to enhance or drive expression of a gene in CNS cells (or in CNS-derived cells) in a preferential or predominant manner as compared to other tissues (e.g. liver, kidney, spleen, heart, muscle and lung). Expression of the gene can be in the form of mRNA or protein. In preferred embodiments, CNS-specific expression is such that there is negligible expression in other (i.e. non-CNS) tissues or cells, i.e. expression is highly CNS-specific.

“PNS-specific” or “PNS-specific expression” refers to the ability of a cis-regulatory element, cis-regulatory module or promoter to enhance or drive expression of a gene in PNS cells (or in PNS-derived cells) in a preferential or predominant manner as compared to other tissues (e.g. liver, kidney, spleen, heart, muscle and lung). Expression of the gene can be in the form of mRNA or protein. In preferred embodiments, PNS-specific expression is such that there is negligible expression in other (i.e. non-PNS) tissues or cells, i.e. expression is highly PNS-specific.

The ability of a CRE, CRM or promoter to function as a NS-specific CRE, CRM or promoter can be readily assessed by the skilled person. The skilled person can thus easily determine whether any variant of the specific CRE, CRM or promoter recited above remains functional (i.e. it is a functional variant as defined above). For example, any given CRM to be assessed can be operably linked to a minimal promoter (e.g. positioned upstream of CMV-MP) and the ability of the cis-regulatory element to drive NS-specific expression of a gene (typically a reporter gene) is measured. Alternatively, a variant of a CRE or CRM can be substituted into a synthetic NS-specific promoter in place of a reference CRE or CRM, and the effects on NS-specific expression driven by said modified promoter can be determined and compared to the unmodified form. Similarly, the ability of a promoter to drive NS-specific expression can be readily assessed by the skilled person (e.g. as described in the examples below). Expression levels of a gene driven by a variant of a reference promoter can be compared to the expression levels driven by the reference promoter. In some embodiments, where NS-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess NS-specific expression enhancement can be easily constructed, and the examples set out below gives suitable methodologies.

NS-specificity can be identified wherein the expression of a gene (e.g. a therapeutic or reporter gene) occurs preferentially or predominantly in NS-derived cells. Preferential or predominant expression can be defined, for example, where the level of expression is significantly greater in NS-derived cells than in other types of cells (i.e. non-NS-derived cells). For example, expression in NS-derived cells is suitably at least 5-fold higher than in non-NS cells, preferably at least 10-fold higher than in non-NS cells, and it may be 50-fold higher or more in some cases. For convenience, NS-specific expression can suitably be demonstrated via a comparison of expression levels in a different non-NS cell lines, e.g. primary NS cells or NS-derived cell line such as SH-Sy5y, Neuro2A, U87-MG compared with expression level in a muscle-derived cell line such as C2C12 or H2K cells (skeletal muscle) or H9C2 cells (cardiac), in a liver-derived cell line (e.g. Huh7 or HepG2), kidney-derived cell line (e.g. HEK-293), a cervical tissue-derived cell line (e.g. HeLa) and/or a lung-derived cell line (e.g. A549).

The synthetic NS-specific promoters of the present invention preferably exhibit reduced expression in non-NS-derived cells, suitably in C2C12, H9C2, Huh7, HEK-293, HeLa, and/or A549 cells when compared to a non-tissue specific promoter such as CMV-IE. The synthetic NS-specific promoters of the present invention preferably have an activity of 50% or less than the CMV-IE promoter in non-NS-derived cells (suitably in C2C12, H9C2, Huh7, HEK-293, HeLa, and/or A549), suitably 25% or less, 20% or less, 15% or less, 10% or less, 5% or less or 1% or less. Generally, it is preferred that expression in non-NS-derived cells is minimized, but in some cases this may not be necessary. Even if a synthetic NS-specific promoter of the present invention has higher expression in, e.g., one or two non-NS cells, as long as it generally has higher expression overall in a range of NS cells versus non-NS cell, it can still be a NS-specific promoter.

The synthetic NS-specific promoters of the present invention are preferably suitable for promoting expression in the NS of a subject, e.g. driving NS-specific expression of a transgene, preferably a therapeutic transgene. Preferred synthetic NS-specific promoters of the present invention are suitable for promoting NS-specific transgene expression and have an activity in NS cells which is at least 15%, 20%, 30%, 40%, 50%, 60%, 70%, 80%, 90%, 100%, 125%, 150%, 175%, 200%, 250%, 300%, 350% or 400% of the activity of the Synapsin-1 promoter. In some embodiments, the synthetic NS-specific promoters of the invention are suitable for promoting NS-specific transgene expression at a level at least 100% of the activity of the Synapsin-1 promoter, preferably 150%, 200%, 300% or 500% of the activity of the Synapsin-1 promoter. Such NS-specific expression is suitably determined in NS-derived cells, e.g. SH-Sy5y, Neuro2A, U87-MG cell lines or primary NS cells (suitably primary human neurones, astrocytes, oligodendrocytes, microglia, Schwann cells, satellite cells and/or ependymal cells).

Synthetic NS-specific promoters of the present invention may also be able to promote NS-specific expression of a gene at a level at least 50%, 100%, 150% or 200% compared to CMV-IE in NS-derived cells, e.g. SH-Sy5y, Neuro2A, U87-MG cell lines or primary NS cells (suitably primary human neurones, astrocytes, oligodendrocytes, microglia, Schwann cells, satellite cells and/or ependymal cells).

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

By “isolated” is meant, when referring to a nucleic acid is a nucleic acid molecule or a nucleic acid sequence devoid, in whole or part, of sequences normally associated with it in nature; or a sequence, as it exists in nature, but having heterologous sequences in association therewith; or a molecule disassociated from the chromosome.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “transcription factor binding site” (TFBS) is well known in the art. It will be apparent to the skilled person that TFBS sequences can be modified, provided that they are bound by the intended transcription factor (TF). Consensus sequences for the various TFBS disclosed herein are known in the art, and the skilled person can readily use this information to determine alternative TFBS. Furthermore, the ability of a TF to bind to a given putative sequence can readily be determined experimentally by the skilled person (e.g. by EMSA and other approaches well known in the art and discussed herein).

The meaning of “consensus sequence” is well-known in the art. In the present application, the following notation is used for the consensus sequences, unless the context dictates otherwise. Considering the following exemplary DNA sequence:
A[CT]N{A}YR

A means that an A is always found in that position; [CT] stands for either C or T in that position; N stands for any base in that position; and {A} means any base except A is found in that position. Y represents any pyrimidine, and R indicates any purine.

“Synthetic” in the present application means a nucleic acid molecule that does not occur in nature. Synthetic nucleic acids of the present invention are produced artificially, typically by recombinant technologies or de novo synthesis. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base.

The term “administration” as used herein refers to introduction of a foreign substance into the human or animal body. Administration can be, for example, intravenous, intraarterial or intracranial.

“Transfection” in the present application refers broadly to any process of deliberately introducing nucleic acids into cells, and covers introduction of viral and non-viral vectors, and includes or is equivalent to transformation, transduction and like terms and processes. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

As used herein, the phrase “transgene” refers to an exogenous nucleic acid sequence. In one example, a transgene is a gene encoding an industrially or pharmaceutically useful compound, or a gene encoding a desirable trait. In yet another example, the transgene encodes useful nucleic acid such as an antisense nucleic acid sequence, wherein expression of the antisense nucleic acid sequence inhibits expression of a target nucleic acid sequence. The transgene preferably encodes a therapeutic product, e.g. a protein.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “operably linked”, “operably connected” or equivalent expressions as used herein refer to the arrangement of various nucleic acid elements relative to each other such that the elements are functionally connected and are able to interact with each other in the manner intended. Such elements may include, without limitation, a promoter, a CRE (e.g. enhancer or other regulatory element), a promoter element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element or their position upstream or downstream of another element or position (such as a TSS or promoter element), and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, CREs will typically be located immediately upstream of the promoter element (although this is generally the case, it should definitely not be interpreted as a limitation or exclusion of positions within the nucleic acid expression cassette), but this needs not be the case in vivo, e.g., a regulatory element sequence naturally occurring downstream of a gene whose transcription it affects is able to function in the same way when located upstream of the promoter. Hence, according to a specific embodiment, the regulatory or enhancing effect of the regulatory element can be position-independent.

A “spacer sequence” or “spacer” as used herein is a nucleic acid sequence that separates two functional nucleic acid sequences (e.g. TFBS, CREs, CRMs, promoter element, etc.). It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another. In some embodiments, spacers may have a length of 75, 50, 40, 30, 30 or 10 nucleotides or fewer.

The term “pharmaceutically acceptable” as used herein is consistent with the art and means compatible with the other ingredients of the pharmaceutical composition and not deleterious to the recipient thereof.

“Therapeutically effective amount” and like phrases mean a dose or plasma concentration in a subject that provides the desired specific pharmacological effect, e.g. to express a therapeutic gene in the NS. A therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The term “AAV vector” as used herein is well known in the art, and generally refers to an AAV vector nucleic acid sequence including various nucleic acid sequences. An AAV vector as used herein typically comprise a heterologous nucleic acid sequence not of AAV origin as part of the vector. This heterologous nucleic acid sequence typically comprises a promoter as disclosed herein as well as other sequences of interest for the genetic transformation of a cell. In general, the heterologous nucleic acid sequence is flanked by at least one, and generally by two AAV inverted terminal repeat sequences (ITRs). An “AAV virion” or “AAV virus” or “AAV viral particle” or “AAV vector particle” refers to a viral particle composed of at least one AAV capsid polypeptide (including both variant AAV capsid polypeptides and non-variant parent capsid polypeptides) and an encapsidated polynucleotide AAV vector. If the particle comprises a heterologous nucleic acid (i.e. a polynucleotide other than a wild-type AAV genome, such as a transgene to be delivered to a mammalian cell), it can be referred to as an “AAV vector particle” or simply an “AAV vector”. Thus, production of AAV virion or AAV particle necessarily includes production of AAV vector as such a vector is contained within an AAV virion or AAV particle. The ITRs may be derived from the same serotype as the capsid, selected from any of the serotypes listed in Table 1, or may be from a different serotype than the capsid. The AAV vector typically has more than one ITR. In a non-limiting example, the AAV vector has a viral genome comprising two ITRs. In one embodiment, the ITRs are of the same serotype as one another. In another embodiment, the ITRs are of different serotypes. Non-limiting examples include zero, one or both of the ITRs having the same serotype as the capsid. Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In one embodiment, the ITRs are 140-142 nucleotides in length. Non-limiting examples of ITR length are 102, 105, 130, 140, 141, 142, 145 nucleotides in length.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome capable of modulating the productive utilization of mRNA. An artificial microRNA can be any type of RNA sequence, other than endogenous microRNA, capable of modulating the activity of an mRNA. A microRNA sequence can be an RNA molecule composed of any one or more of these sequences. MicroRNA (or “miRNA”) sequences have been described in publications such as Lim, et al, 2003, Genes & Development, 17, 991-1008, Lim et al, 2003, Science, 299, 1540, Lee and Ambrose, 2001, Science, 294, 862, Lau et al, 2001, Science 294, 858-861, Lagos-Quintana et al, 2002, Current Biology, 12, 735-739, Lagos-Quintana ei a/., 2001, Science, 294, 853-857, and Lagos-Quintana et al., 2003, RNA, 9, 175-179. Examples of microRNAs include any RNA fragment of a larger RNA or is a miRNA, siRNA, stRNA, sncRNA, tncRNA, snoRNA, smRNA, shRNA, snRNA, or other small non-coding RNA. See, e.g., US Patent Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA cleaved from a microRNA precursor (a “pre-miRNA”), or synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

The terms “treatment” or “treating” refer to reducing, ameliorating or eliminating one or more signs, symptoms, or effects of a disease or condition. “Treatment,” as used herein thus includes any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

The “administration” of an agent to a subject includes any route of introducing or delivering to a subject the agent to perform its intended function. Administration can be carried out by any suitable route, including orally, intranasally, intraocularly, ophthalmically, parenterally (intravascularly, intramuscularly, intraperitoneally, or subcutaneously), or topically. Administration includes self-administration and the administration by another. Intravenous or intraarterial administration is of particular interest in the present invention.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

The term “specifically active in an area or in a tissue” refers to a promoter which is predominantly active in that area or tissue, i.e. more active in that area or tissue than in other areas or tissues.

### EXAMPLE 1

### Sequence Information

TABLE 1
NS-specific promoters

| NAME | SEQUENCE | Length |
|---|---|---|
| SP0013 | AACATATCCTGGTGTGGAGTAGGGGACGCTGCTCTGACAGAGGCTCGGGGG | 795 |
| (SEQ ID | CCTGAGCTGGCTCTGTGAGCTGGGGAGGAGGCAGACAGCCAGGCCTTGTCT | |
| NO: 1) | GCAAGCAGACCTGGCAGCATTGGGCTGGCCGCCCCCCAGGGCCTCCTCTTC | |
| | ATGCCCAGTGAATGACTCACCTTGGCACAGACACAATGTTCGGGGTGGGCA | |
| | CAGTGCCTGCTTCCCGCCGCACCCCAGCCCCCCTCAAATGCCTTCCGAGAA | |
| | GCCCATTGAGCAGGGGGCTTGCATTGCACCCCAGCCTGACAGCCTGGCATC | |
| | TTGGGATAAAAGCAGCACAGCCCCCTAGGGGCTGCCCTTGCTGTGTGGCGC | |
| | CACCGGCGGTGGAGAACAAGGCTCTATTCAGCCTGTGCCCAGGAAAGGGGA | |
| | TCAGGGGATGCCCAGGCATGGACAGTGGGTGGCAGGGGGGGAGAGGAGG | |
| | GCTGTCTGCTTCCCAGAAGTCCAAGGACACAAATGGGTGAGGGGAGGGCTA | |
| | GGCCTGCGCACCCACCCACCGACCCCTCACCCACCGACCCGTCACCCACCG | |
| | ACCAAGGGGCACCCTGGCCTAGAGGGGATGCTGAGCGGGACCCGCCTCCT | |
| | GCCTCTGGCAGTCCCAGATGGGACTTGGACCCCGCAGTTGCTCTCTCGGAC | |
| | CCTAAGTTTCTACCCCTGGATCTAAGGCGGAGCTGGGTTTGCGGATCCCACG | |
| | GTTCCCGGCGGGGCGGGGCCCGGTCGCCCCTCCCCCTCCCCGCCCTCCTG | |
| | CGCCGGGAGCAGTGCATTGTGGGAAACTCCCGA | |
| SP0014 | AACATATCCTGGTGTGGAGTAGGGGACGCTGCTCTGACAGAGGCTCGGGGG | 810 |
| (SEQ ID | CCTGAGCTGGCTCTGTGAGCTGGGGAGGAGGCAGACAGCCAGGCCTTGTCT | |
| NO: 2) | GCAAGCAGACCTGGCAGCATTGGGCTGGCCGCCCCCCAGGGCCTCCTCTTC | |
| | ATGCCCAGTGAATGACTCACCTTGGCACAGACACAATGTTCGGGGTGGGCA | |
| | CAGTGCCTGCTTCCCGCCGCACCCCAGCCCCCCTCAAATGCCTTCCGAGAA | |
| | GCCCATTGAGCAGGGGGCTTGCATTGCACCCCAGCCTGACAGCCTGGCATC | |
| | TTGGGATAAAAGCAGCACAGCCCCCTAGGGGCTGCCCTTGCTGTGTGGCGC | |
| | CACCGGCGGTGGAGAACAAGGCTCTATTCAGCCTGTGCCCAGGAAAGGGGA | |
| | TCAGGGGATGCCCAGGCATGGACAGTGGGTGGCAGGGGGGGAGAGGAGG | |
| | GCTGTCTGCTTCCCAGAAGTCCAAGGACACAAATGGGTGAGGGGATGCGGC | |
| | GAGGCGCGTGCGCACTGCCAGCTTCAGCACCGCGGACAGTGCCTTCGCCC | |
| | CCGCCTGGCGGCGCGCGCCACCGCCGCCTCAGCACTGAAGGCGCGCTGAC | |
| | GTCACTCGCCGGTCCCCCGCAAACTCCCCTTCCCGGCCACCTTGGTCGCGT | |
| | CCGCGCCGCCGCCGGCCCAGCCGGACCGCACCACGCGAGGCGCGAGATA | |
| | GGGGGGCACGGGCGCGACCATCTGCGCTGCGGCGCCGGCGACTCAGCGC | |
| | TGCCTCAGTCTGCGGTGGGCAGCGGAGGAGTCGTGTCGTGCCTGAGAGCG | |
| | CAG | |
| SP0026 | AACATATCCTGGTGTGGAGTAGGGGACGCTGCTCTGACAGAGGCTCGGGGG | 916 |
| (SEQ ID | CCTGAGCTGGCTCTGTGAGCTGGGGAGGAGGCAGACAGCCAGGCCTTGTCT | |
| NO: 3) | GCAAGCAGACCTGGCAGCATTGGGCTGGCCGCCCCCCAGGGCCTCCTCTTC | |
| | ATGCCCAGTGAATGACTCACCTTGGCACAGACACAATGTTCGGGGTGGGCA | |
| | CAGTGCCTGCTTCCCGCCGCACCCCAGCCCCCCTCAAATGCCTTCCGAGAA | |
| | GCCCATTGAGCAGGGGGCTTGCATTGCACCCCAGCCTGACAGCCTGGCATC | |
| | TTGGGATAAAAGCAGCACAGCCCCCTAGGGGCTGCCCTTGCTGTGTGGCGC | |
| | CACCGGCGGTGGAGAACAAGGCTCTATTCAGCCTGTGCCCAGGAAAGGGGA | |
| | TCAGGGGATGCCCAGGCATGGACAGTGGGTGGCAGGGGGGGAGAGGAGG | |
| | GCTGTCTGCTTCCCAGAAGTCCAAGGACACAAATGGGTGAGGGGAAGCGCG | |
| | CAGAGTCTGCATGCGTGAGGAAGCTCCTGGGCGCGTCACAGCCGCGCTATT | |
| | CTCAGCGTCTCTCCTTTTATGGCTCCGGAAGTGAGCTGGGGTTGCTGGCAG | |
| | CCTGGCTGGCACTGGGCTAGGCCTGCGCACCCACCCACCGACCCCTCACCC | |
| | ACCGACCCGTCACCCACCGACCAAGGGGCACCCTGGCCTAGAGGGGATGC | |
| | TGAGCGGGACCCGCCTCCTGCCTCTGGCAGTCCCAGATGGGACTTGGACCC | |
| | CGCAGTTGCTCTCTCGGACCCTAAGTTTCTACCCCTGGATCTAAGGCGGAGC | |
| | TGGGTTTGCGGATCCCACGGTTCCCGGCGGGGCGGGGCCCGGTCGCCCCT | |
| | CCCCCTCCCCGCCCTCCTGCGCCGGGAGCAGTGCATTGTGGGAAACTCCCG | |
| | A | |
| SP0027 | AACATATCCTGGTGTGGAGTAGGGGACGCTGCTCTGACAGAGGCTCGGGGG | 931 |
| (SEQ ID | CCTGAGCTGGCTCTGTGAGCTGGGGAGGAGGCAGACAGCCAGGCCTTGTCT | |
| NO: 4) | GCAAGCAGACCTGGCAGCATTGGGCTGGCCGCCCCCCAGGGCCTCCTCTTC | |
| | ATGCCCAGTGAATGACTCACCTTGGCACAGACACAATGTTCGGGGTGGGCA | |
| | CAGTGCCTGCTTCCCGCCGCACCCCAGCCCCCCTCAAATGCCTTCCGAGAA | |
| | GCCCATTGAGCAGGGGGCTTGCATTGCACCCCAGCCTGACAGCCTGGCATC | |
| | TTGGGATAAAAGCAGCACAGCCCCCTAGGGGCTGCCCTTGCTGTGTGGCGC | |
| | CACCGGCGGTGGAGAACAAGGCTCTATTCAGCCTGTGCCCAGGAAAGGGGA | |
| | TCAGGGGATGCCCAGGCATGGACAGTGGGTGGCAGGGGGGGAGAGGAGG | |
| | GCTGTCTGCTTCCCAGAAGTCCAAGGACACAAATGGGTGAGGGGAAGCGCG | |
| | CAGAGTCTGCATGCGTGAGGAAGCTCCTGGGCGCGTCACAGCCGCGCTATT | |
| | CTCAGCGTCTCTCCTTTTATGGCTCCGGAAGTGAGCTGGGGTTGCTGGCAG | |
| | CCTGGCTGGCACTTGCGGCGAGGCGCGTGCGCACTGCCAGCTTCAGCACC | |
| | GCGGACAGTGCCTTCGCCCCCGCCTGGCGGCGCGCGCCACCGCCGCCTCA | |
| | GCACTGAAGGCGCGCTGACGTCACTCGCCGGTCCCCCGCAAACTCCCCTTC | |
| | CCGGCCACCTTGGTCGCGTCCGCGCCGCCGCCGGCCCAGCCGGACCGCAC | |
| | CACGCGAGGCGCGAGATAGGGGGGCACGGGCGCGACCATCTGCGCTGCG | |
| | GCGCCGGCGACTCAGCGCTGCCTCAGTCTGCGGTGGGCAGCGGAGGAGTC | |
| | GTGTCGTGCCTGAGAGCGCAG | |
| SP0030 | AACATATCCTGGTGTGGAGTAGGGGACGCTGCTCTGACAGAGGCTCGGGGG | 617 |
| (SEQ ID | CCTGAGCTGGCTCTGTGAGCTGGGGAGGAGGCAGACAGCCAGGCCTTGTCT | |
| NO: 5) | GCAAGCAGACCTGGCAGCATTGGGCTGGCCGCCCCCCAGGGCCTCCTCTTC | |
| | ATGCCCAGTGAATGACTCACCTTGGCACAGACACAATGTTCGGGGTGGGCA | |
| | CAGTGCCTGCTTCCCGCCGCACCCCAGCCCCCCTCAAATGCCTTCCGAGAA | |
| | GCCCATTGAGCAGGGGGCTTGCATTGCACCCCAGCCTGACAGCCTGGCATC | |
| | TTGGGATAAAAGCAGCACGGGCTAGGCCTGCGCACCCACCCACCGACCCCT | |
| | CACCCACCGACCCGTCACCCACCGACCAAGGGGCACCCTGGCCTAGAGGG | |
| | GATGCTGAGCGGGACCCGCCTCCTGCCTCTGGCAGTCCCAGATGGGACTTG | |
| | GACCCCGCAGTTGCTCTCTCGGACCCTAAGTTTCTACCCCTGGATCTAAGGC | |
| | GGAGCTGGGTTTGCGGATCCCACGGTTCCCGGCGGGGCGGGGCCCGGTCG | |
| | CCCCTCCCCCTCCCCGCCCTCCTGCGCCGGGAGCAGTGCATTGTGGGAAAC | |
| | TCCCGA | |
| SP0031 | AACATATCCTGGTGTGGAGTAGGGGACGCTGCTCTGACAGAGGCTCGGGGG | 632 |
| (SEQ ID | CCTGAGCTGGCTCTGTGAGCTGGGGAGGAGGCAGACAGCCAGGCCTTGTCT | |
| NO: 6) | GCAAGCAGACCTGGCAGCATTGGGCTGGCCGCCCCCCAGGGCCTCCTCTTC | |
| | ATGCCCAGTGAATGACTCACCTTGGCACAGACACAATGTTCGGGGTGGGCA | |
| | CAGTGCCTGCTTCCCGCCGCACCCCAGCCCCCCTCAAATGCCTTCCGAGAA | |
| | GCCCATTGAGCAGGGGGCTTGCATTGCACCCCAGCCTGACAGCCTGGCATC | |
| | TTGGGATAAAAGCAGCACTGCGGCGAGGCGCGTGCGCACTGCCAGCTTCAG | |
| | CACCGCGGACAGTGCCTTCGCCCCCGCCTGGCGGCGCGCGCCACCGCCGC | |
| | CTCAGCACTGAAGGCGCGCTGACGTCACTCGCCGGTCCCCCGCAAACTCCC | |
| | CTTCCCGGCCACCTTGGTCGCGTCCGCGCCGCCGCCGGCCCAGCCGGACC | |
| | GCACCACGCGAGGCGCGAGATAGGGGGGCACGGGCGCGACCATCTGCGCT | |
| | GCGGCGCCGGCGACTCAGCGCTGCCTCAGTCTGCGGTGGGCAGCGGAGGA | |
| | GTCGTGTCGTGCCTGAGAGCGCAG | |
| SP0032 | TTCAGAGGTCGGATCTGAATCCAGCTCCAAGGCCCCAGCACCCAAGCCCTG | 701 |
| (SEQ ID | ACCCCATGGCTGCCTGCTGGCTGGGAGTGGCATTCTTTAGAACAGGAAAGG | |
| NO: 7) | CAGTGAGTTCTCATTGCATCAATACTTGCATTTGCTACAACAGAAGCTTTTTG | |
| | TCTCTTCCTCACATTCCTTTAGAACACAAGCCTCCTTTTCTGCCTGTGGCCAT | |
| | CGGTCACTGGAGTCAGCCTCGTGAGTGGCTTGGTGGCGGATGGCACCGTCC | |
| | TTTGTGGGAGAAAACAATGTTGCTGCCCAGGCCTTTCTGGAATGACCCCTTC | |
| | CCACCCCCTGACCAGCCCCAGCAAGGCCCGGGGCTGGCTGCCTAGTTGATT | |
| | GGAGCCAAGAGTTTGCTGAATGGATGAAGGGAGAAGGGACATCCAAGGGCT | |
| | AGGCCTGCGCACCCACCCACCGACCCCTCACCCACCGACCCGTCACCCACC | |
| | GACCAAGGGGCACCCTGGCCTAGAGGGGATGCTGAGCGGGACCCGCCTCC | |
| | TGCCTCTGGCAGTCCCAGATGGGACTTGGACCCCGCAGTTGCTCTCTCGGA | |
| | CCCTAAGTTTCTACCCCTGGATCTAAGGCGGAGCTGGGTTTGCGGATCCCAC | |
| | GGTTCCCGGCGGGGCGGGGCCCGGTCGCCCCTCCCCCTCCCCGCCCTCCT | |
| | GCGCCGGGAGCAGTGCATTGTGGGAAACTCCCGA | |
| SP0033 | TTCAGAGGTCGGATCTGAATCCAGCTCCAAGGCCCCAGCACCCAAGCCCTGACCCCATG | 716 |
| (SEQ ID | GCTGCCTGCTGGCTGGGAGTGGCATTCTTTAGAACAGGAAAGGCAGTGAGTTCTCATT | |
| NO: 8) | GCATCAATACTTGCATTTGCTACAACAGAAGCTTTTTGTCTCTTCCTCACATTCCTTTAGA | |
| | ACACAAGCCTCCTTTTCTGCCTGTGGCCATCGGTCACTGGAGTCAGCCTCGTGAGTGGC | |
| | TTGGTGGCGGATGGCACCGTCCTTTGTGGGAGAAAACAATGTTGCTGCCCAGGCCTTTC | |
| | TGGAATGACCCCTTCCCACCCCCTGACCAGCCCCAGCAAGGCCCGGGGCTGGCTGCCTA | |
| | GTTGATTGGAGCCAAGAGTTTGCTGAATGGATGAAGGGAGAAGGGACATCCAATGCG | |
| | GCGAGGCGCGTGCGCACTGCCAGCTTCAGCACCGCGGACAGTGCCTTCGCCCCCGCCT | |
| | GGCGGCGCGCGCCACCGCCGCCTCAGCACTGAAGGCGCGCTGACGTCACTCGCCGGTC | |
| | CCCCGCAAACTCCCCTTCCCGGCCACCTTGGTCGCGTCCGCGCCGCCGCCGGCCCAGCC | |
| | GGACCGCACCACGCGAGGCGCGAGATAGGGGGGCACGGGCGCGACCATCTGCGCTGC | |
| | GGCGCCGGCGACTCAGCGCTGCCTCAGTCTGCGGTGGGCAGCGGAGGAGTCGTGTCG | |
| | TGCCTGAGAGCGCAG | |
| SP0019 | TCAGGGGTGCAGCTTTTTTTCTGTCTTTTACTCAGCCTGAGAAAGGTTGTCGTTTGACAA | 792 |
| (SEQ ID | GGTTTGTTCAGAGGTCGGATCTGAATCCAGCTCCAAGGCCCCAGCACCCAAGCCCTGAC | |
| NO: 9) | CCCATGGCTGCCTGCTGGCTGGGAGTGGCATTCTTTAGAACAGGAAAGGCAGTGAGTT | |
| | CTCATTGCATCAATACTTGCATTTGCTACAACAGAAGCTTTTTGTCTCTTCCTCACATTCC | |
| | TTTAGAACACAAGCCTCCTTTTCTGCCTGTGGCCATCGGTCACTGGAGTCAGCCTCGTGA | |
| | GTGGCTTGGTGGCGGATGGCACCGTCCTTTGTGGGAGAAAACAATGTTGCTGCCCAGG | |
| | CCTTTCTGGAATGACCCCTTCCCACCCCCTGACCAGCCCCAGCAAGGCCCGGGGCTGGC | |
| | TGCCTAGTTGATTGGAGCCAAGAGTTTGCTGAATGGATGAAGGGAGAAGGGACATCCA | |
| | AGTGACTGAAGAAGATCTTAACAGAAGGGCTAGGCCTGCGCACCCACCCACCGACCCC | |
| | TCACCCACCGACCCGTCACCCACCGACCAAGGGGCACCCTGGCCTAGAGGGGATGCTG | |
| | AGCGGGACCCGCCTCCTGCCTCTGGCAGTCCCAGATGGGACTTGGACCCCGCAGTTGCT | |
| | CTCTCGGACCCTAAGTTTCTACCCCTGGATCTAAGGCGGAGCTGGGTTTGCGGATCCCA | |
| | CGGTTCCCGGCGGGGCGGGGCCCGGTCGCCCCTCCCCCTCCCCGCCCTCCTGCGCCGG | |
| | GAGCAGTGCATTGTGGGAAACTCCCGA | |
| SP0020 | CTTCCTCTTATATTTCACCAAGACTCAGTTCCTGAGCAAGAAACCACAGGCACAGCAAGT | 686 |
| (SEQ ID | GCCATGAAAAGCGGCTTTGTGTGGGGTGGGCTCTTCACACTCCAATCTCCACTTCCTTCT | |
| NO: 10) | CAAGGCCTCAAAAAAAGTTGAAAAATGAAAACAAAAGCCCTGCTGTGTTGAGCTGGGC | |
| | TCTGGCGTTGCCATGGACCCAGGGCAAACAGCGGTGCTCCTGCTCTGCCCCCGGCTCAG | |
| | CTCATGCTGGGCCTGCACTTCTGGAAGGGAGCATGGACTTTGGAATGACTGGTTAGAA | |
| | CCCAAATGAATTAATGGAATTTGACATAGTTCAAAAATAATAAAATGTGATACCCATGA | |
| | AATGCTGATATTCTGCCTTAATTTGCCAGATTGGGGGCCGGGCTAGGCCTGCGCACCCA | |
| | CCCACCGACCCCTCACCCACCGACCCGTCACCCACCGACCAAGGGGCACCCTGGCCTAG | |
| | AGGGGATGCTGAGCGGGACCCGCCTCCTGCCTCTGGCAGTCCCAGATGGGACTTGGAC | |
| | CCCGCAGTTGCTCTCTCGGACCCTAAGTTTCTACCCCTGGATCTAAGGCGGAGCTGGGT | |
| | TTGCGGATCCCACGGTTCCCGGCGGGGCGGGGCCCGGTCGCCCCTCCCCCTCCCCGCCC | |
| | TCCTGCGCCGGGAGCAGTGCATTGTGGGAAACTCCCGA | |
| SP0021 | TCAGGGGTGCAGCTTTTTTTCTGTCTTTTACTCAGCCTGAGAAAGGTTGTCGTTTGACAA | 807 |
| (SEQ ID | GGTTTGTTCAGAGGTCGGATCTGAATCCAGCTCCAAGGCCCCAGCACCCAAGCCCTGAC | |
| NO: 11) | CCCATGGCTGCCTGCTGGCTGGGAGTGGCATTCTTTAGAACAGGAAAGGCAGTGAGTT | |
| | CTCATTGCATCAATACTTGCATTTGCTACAACAGAAGCTTTTTGTCTCTTCCTCACATTCC | |
| | TTTAGAACACAAGCCTCCTTTTCTGCCTGTGGCCATCGGTCACTGGAGTCAGCCTCGTGA | |
| | GTGGCTTGGTGGCGGATGGCACCGTCCTTTGTGGGAGAAAACAATGTTGCTGCCCAGG | |
| | CCTTTCTGGAATGACCCCTTCCCACCCCCTGACCAGCCCCAGCAAGGCCCGGGGCTGGC | |
| | TGCCTAGTTGATTGGAGCCAAGAGTTTGCTGAATGGATGAAGGGAGAAGGGACATCCA | |
| | AGTGACTGAAGAAGATCTTAACAGAATGCGGCGAGGCGCGTGCGCACTGCCAGCTTCA | |
| | GCACCGCGGACAGTGCCTTCGCCCCCGCCTGGCGGCGCGCGCCACCGCCGCCTCAGCA | |
| | CTGAAGGCGCGCTGACGTCACTCGCCGGTCCCCCGCAAACTCCCCTTCCCGGCCACCTT | |
| | GGTCGCGTCCGCGCCGCCGCCGGCCCAGCCGGACCGCACCACGCGAGGCGCGAGATA | |
| | GGGGGGCACGGGCGCGACCATCTGCGCTGCGGCGCCGGCGACTCAGCGCTGCCTCAG | |
| | TCTGCGGTGGGCAGCGGAGGAGTCGTGTCGTGCCTGAGAGCGCAG | |
| SP0022 | CTTCCTCTTATATTTCACCAAGACTCAGTTCCTGAGCAAGAAACCACAGGCACAGCAAGT | 701 |
| (SEQ ID | GCCATGAAAAGCGGCTTTGTGTGGGGTGGGCTCTTCACACTCCAATCTCCACTTCCTTCT | |
| NO: 12) | CAAGGCCTCAAAAAAAGTTGAAAAATGAAAACAAAAGCCCTGCTGTGTTGAGCTGGGC | |
| | TCTGGCGTTGCCATGGACCCAGGGCAAACAGCGGTGCTCCTGCTCTGCCCCCGGCTCAG | |
| | CTCATGCTGGGCCTGCACTTCTGGAAGGGAGCATGGACTTTGGAATGACTGGTTAGAA | |
| | CCCAAATGAATTAATGGAATTTGACATAGTTCAAAAATAATAAAATGTGATACCCATGA | |
| | AATGCTGATATTCTGCCTTAATTTGCCAGATTGGGGGCCTGCGGCGAGGCGCGTGCGC | |
| | ACTGCCAGCTTCAGCACCGCGGACAGTGCCTTCGCCCCCGCCTGGCGGCGCGCGCCACC | |
| | GCCGCCTCAGCACTGAAGGCGCGCTGACGTCACTCGCCGGTCCCCCGCAAACTCCCCTT | |
| | CCCGGCCACCTTGGTCGCGTCCGCGCCGCCGCCGGCCCAGCCGGACCGCACCACGCGA | |
| | GGCGCGAGATAGGGGGGCACGGGCGCGACCATCTGCGCTGCGGCGCCGGCGACTCAG | |
| | CGCTGCCTCAGTCTGCGGTGGGCAGCGGAGGAGTCGTGTCGTGCCTGAGAGCGCAG | |
| SP0028 | TCAGGGGTGCAGCTTTTTTTCTGTCTTTTACTCAGCCTGAGAAAGGTTGTCGTTTGACAA | 913 |
| (SEQ ID | GGTTTGTTCAGAGGTCGGATCTGAATCCAGCTCCAAGGCCCCAGCACCCAAGCCCTGAC | |
| NO: 13) | CCCATGGCTGCCTGCTGGCTGGGAGTGGCATTCTTTAGAACAGGAAAGGCAGTGAGTT | |
| | CTCATTGCATCAATACTTGCATTTGCTACAACAGAAGCTTTTTGTCTCTTCCTCACATTCC | |
| | TTTAGAACACAAGCCTCCTTTTCTGCCTGTGGCCATCGGTCACTGGAGTCAGCCTCGTGA | |
| | GTGGCTTGGTGGCGGATGGCACCGTCCTTTGTGGGAGAAAACAATGTTGCTGCCCAGG | |
| | CCTTTCTGGAATGACCCCTTCCCACCCCCTGACCAGCCCCAGCAAGGCCCGGGGCTGGC | |
| | TGCCTAGTTGATTGGAGCCAAGAGTTTGCTGAATGGATGAAGGGAGAAGGGACATCCA | |
| | AGTGACTGAAGAAGATCTTAACAGAAAGCGCGCAGAGTCTGCATGCGTGAGGAAGCTC | |
| | CTGGGCGCGTCACAGCCGCGCTATTCTCAGCGTCTCTCCTTTTATGGCTCCGGAAGTGA | |
| | GCTGGGGTTGCTGGCAGCCTGGCTGGCACTGGGCTAGGCCTGCGCACCCACCCACCGA | |
| | CCCCTCACCCACCGACCCGTCACCCACCGACCAAGGGGCACCCTGGCCTAGAGGGGAT | |
| | GCTGAGCGGGACCCGCCTCCTGCCTCTGGCAGTCCCAGATGGGACTTGGACCCCGCAG | |
| | TTGCTCTCTCGGACCCTAAGTTTCTACCCCTGGATCTAAGGCGGAGCTGGGTTTGCGGA | |
| | TCCCACGGTTCCCGGCGGGGCGGGGCCCGGTCGCCCCTCCCCCTCCCCGCCCTCCTGCG | |
| | CCGGGAGCAGTGCATTGTGGGAAACTCCCGA | |
| SP0029 | TCAGGGGTGCAGCTTTTTTTCTGTCTTTTACTCAGCCTGAGAAAGGTTGTCGTTTGACAA | 928 |
| (SEQ ID | GGTTTGTTCAGAGGTCGGATCTGAATCCAGCTCCAAGGCCCCAGCACCCAAGCCCTGAC | |
| NO: 14) | CCCATGGCTGCCTGCTGGCTGGGAGTGGCATTCTTTAGAACAGGAAAGGCAGTGAGTT | |
| | CTCATTGCATCAATACTTGCATTTGCTACAACAGAAGCTTTTTGTCTCTTCCTCACATTCC | |
| | TTTAGAACACAAGCCTCCTTTTCTGCCTGTGGCCATCGGTCACTGGAGTCAGCCTCGTGA | |
| | GTGGCTTGGTGGCGGATGGCACCGTCCTTTGTGGGAGAAAACAATGTTGCTGCCCAGG | |
| | CCTTTCTGGAATGACCCCTTCCCACCCCCTGACCAGCCCCAGCAAGGCCCGGGGCTGGC | |
| | TGCCTAGTTGATTGGAGCCAAGAGTTTGCTGAATGGATGAAGGGAGAAGGGACATCCA | |
| | AGTGACTGAAGAAGATCTTAACAGAAAGCGCGCAGAGTCTGCATGCGTGAGGAAGCTC | |
| | CTGGGCGCGTCACAGCCGCGCTATTCTCAGCGTCTCTCCTTTTATGGCTCCGGAAGTGA | |
| | GCTGGGGTTGCTGGCAGCCTGGCTGGCACTTGCGGCGAGGCGCGTGCGCACTGCCAGC | |
| | TTCAGCACCGCGGACAGTGCCTTCGCCCCCGCCTGGCGGCGCGCGCCACCGCCGCCTCA | |
| | GCACTGAAGGCGCGCTGACGTCACTCGCCGGTCCCCCGCAAACTCCCCTTCCCGGCCAC | |
| | CTTGGTCGCGTCCGCGCCGCCGCCGGCCCAGCCGGACCGCACCACGCGAGGCGCGAGA | |
| | TAGGGGGGCACGGGCGCGACCATCTGCGCTGCGGCGCCGGCGACTCAGCGCTGCCTC | |
| | AGTCTGCGGTGGGCAGCGGAGGAGTCGTGTCGTGCCTGAGAGCGCAG | |
| SP0011 | CTAGCCCACAGGAAATGTCTGTCTATATCCAGGCAAGTACCTTGCTCATTGGACCAACCC | 529 |
| (SEQ ID | GAAACTGTTCAGGGAAGATCAGGGAAATCAACTCAGTTACAAATGGGATAATCATGCC | |
| NO: 33) | CAGTAAAAACTACCTGTGGTGAATAAAGAGTTAACCCCTGTTCCATCTTAGGTCACTATG | |
| | CAGAGTACCAATGAGTACAAGAGATGGTGCCAAAGAGGGTGGCCCCTCCCTAGCTGGG | |
| | AACAGTCAACCCTTAGGAACTAGACTGTCAACACATCAGCCAGCCAGAGACAAGGGAA | |
| | ACCGTGGCAACCAAGTGTTGCTGGCACATTGTGAGGTGGTGATGGGAACTGCAGAGGC | |
| | CCTGCACAGCATGCTAATGAGCCCAGGCAAACATGAGCTCTCCCCATAGCTGGGCTGCG | |
| | GCCCAACCCCACCCCCTCAGGCTATGCCAGGGGGTGTTGCCAGGGGCACCCGGGCATC | |
| | GCCAGTCTAGCCCACTCCTTCATAAAGCCCTCGCATCCCAGGAGCGAGCAGAGCCAGAG | |
| | C | |
| SP0034 | TCCAAAGAAAAGCCAGATAAGTAGCTGATTATTGCATAGAGCTGACAGTATCACAGGA | 646 |
| (SEQ ID | AGATCAGTAGTAGCAGCTCAAGTACAAAAAGGTTAATTAGCAATACTTAATAAGAAAAA | |
| NO: 34) | CTACCTCTGGCAGGTGAAGAGTTAATCCCTGGTCAATTTTAAGCTACTCTGCTGAGAGT | |
| | ACTAATAAGTGTAGGGGTTGGAGCCAATGAGGGTGACCCCTTCCTTGATGGGAACAGT | |
| | CATCCCTTAGGAACTGCCCTGGAAAGCATCAGCCAGCCAGAAAACAGGGAAAGAGGCT | |
| | GAGAAACCGTGGTAACCAAGTTTTGCTGGCACTTTGTAAAATGGTAACTGCAACTGCCG | |
| | AGGCTGTGCAGAGAATGCTAATAAGCCTAGGACAACCTGTAAAGAGTGGACCTAGAAA | |
| | ATGTCCACCCGCTAGAGAGAGGGAGCGAGCATGTGCGATGAGCAATAGCTGTGGACCT | |
| | TACAGTTGCTGCTAACTGCCCTGGTGTGTGTGAGGGAGAGAGAGGGAGGGAGGGAGA | |
| | GAGAGCGCGCTAGCGCGAGAGAGCGAGTGAGCAAGCGAGCAGAAAAGAGGTGGAGA | |
| | GGGGGGGAATAAGAAAGAGAGAGAAGGAAAGGAGAGAAGGCAGGAAGAAGGCAAG | |
| | GGACGAGACAA | |
| SP0035 | CTAGCCCACAGGAAATGTCTGTCTATATCCAGGCAAGTACCTTGCTCATTGGACCAACCC | 614 |
| (SEQ ID | GAAACTGTTCAGGGAAGATCAGGGAAATCAACTCAGTTACAAATGGGATAATCATGCC | |
| NO: 35) | CAGTAAAAACTACCTGTGGTGAATAAAGAGTTAACCCCTGTTCCATCTTAGGTCACTATG | |
| | CAGAGTACCAATGAGTACAAGAGATGGTGCCAAAGAGGGTGGCCCCTCCCTAGCTGGG | |
| | AACAGTCAACCCTTAGGAACTAGACTGTCAACACATCAGCCAGCCAGAGACAAGGGAA | |
| | ACCGTGGCAACCAAGTGTTGCTGGCACATTGTGAGGTGGTGATGGGAACTGCAGAGGC | |
| | CCTGCACAGCATGCTAATGAGCCCAGGCAAACATCGCTAGAGAGAGGGAGCGAGCAT | |
| | GTGCGATGAGCAATAGCTGTGGACCTTACAGTTGCTGCTAACTGCCCTGGTGTGTGTGA | |
| | GGGAGAGAGAGGGAGGGAGGGAGAGAGAGCGCGCTAGCGCGAGAGAGCGAGTGAG | |
| | CAAGCGAGCAGAAAAGAGGTGGAGAGGGGGGGAATAAGAAAGAGAGAGAAGGAAA | |
| | GGAGAGAAGGCAGGAAGAAGGCAAGGGACGAGACAA | |
| SP0036 | CTAGCCCACAGGAAATGTCTGTCTATATCCAGGCAAGTACCTTGCTCATTGGACCAACCC | 695 |
| (SEQ ID | GAAACTGTTCAGGGAAGATCAGGGAAATCAACTCAGTTACAAATGGGATAATCATGCC | |
| NO: 36) | CAGTAAAAACTACCTGTGGTGAATAAAGAGTTAACCCCTGTTCCATCTTAGGTCACTATG | |
| | CAGAGTACCAATGAGTACAAGAGATGGTGCCAAAGAGGGTGGCCCCTCCCTAGCTGGG | |
| | AACAGTCAACCCTTAGGAACTAGACTGTCAACACATCAGCCAGCCAGAGACAAGGGAA | |
| | ACCGTGGCAACCAAGTGTTGCTGGCACATTGTGAGGTGGTGATGGGAACTGCAGAGGC | |
| | CCTGCACAGCATGCTAATGAGCCCAGGCAAACATTCGAGTTGGCTGGACAAGGTTATG | |
| | AGCATCCGTGTACTTATGGGGTTGCCAGCTTGGTCCTGGATCGCCCGGGCCCTTCCCCC | |
| | ACCCGTTCGGTTCCCCACCACCACCCGCGCTCGTACGTGCGTCTCCGCCTGCAGCTCTTG | |
| | ACTCATCGGGGCCCCCCGGGTCACATGCGCTCGCTCGGCTCTATAGGCGCCGCCCCCTG | |
| | CCCACCCCCCGCCCGCGCTGGGAGCCGCAGCCGCCGCCACTCCTGCTCTCTCTGCGCCG | |
| | CCGCCGTCACCACCGCCACCGCCACCGGCTGAGTCTGCAGTCCTCGAG | |

TABLE 2
CRMs from promoters of Table 1

| Name | SEQUENCE |
|---|---|
| CRM_SP0026 | AACATATCCTGGTGTGGAGTAGGGGACGCTGCTCTGACAGAGGCTCGGGGGC |
| (SEQ ID NO: 15) | CTGAGCTGGCTCTGTGAGCTGGGGAGGAGGCAGACAGCCAGGCCTTGTCTGC |
| | AAGCAGACCTGGCAGCATTGGGCTGGCCGCCCCCCAGGGCCTCCTCTTCATG |
| | CCCAGTGAATGACTCACCTTGGCACAGACACAATGTTCGGGGTGGGCACAGT |
| | GCCTGCTTCCCGCCGCACCCCAGCCCCCCTCAAATGCCTTCCGAGAAGCCCA |
| | TTGAGCAGGGGGCTTGCATTGCACCCCAGCCTGACAGCCTGGCATCTTGGGA |
| | TAAAAGCAGCACAGCCCCCTAGGGGCTGCCCTTGCTGTGTGGCGCCACCGGC |
| | GGTGGAGAACAAGGCTCTATTCAGCCTGTGCCCAGGAAAGGGGATCAGGGGA |
| | TGCCCAGGCATGGACAGTGGGTGGCAGGGGGGGAGAGGAGGGCTGTCTGCT |
| | TCCCAGAAGTCCAAGGACACAAATGGGTGAGGGGAAGCGCGCAGAGTCTGCA |
| | TGCGTGAGGAAGCTCCTGGGCGCGTCACAGCCGCGCTATTCTCAGCGTCTCT |
| | CCTTTTATGGCTCCGGAAGTGAGCTGGGGTTGCTGGCAGCCTGGCTGGCACT |
| CRM_SP0027 | AACATATCCTGGTGTGGAGTAGGGGACGCTGCTCTGACAGAGGCTCGGGGGC |
| (SEQ ID NO: 16) | CTGAGCTGGCTCTGTGAGCTGGGGAGGAGGCAGACAGCCAGGCCTTGTCTGC |
| | AAGCAGACCTGGCAGCATTGGGCTGGCCGCCCCCCAGGGCCTCCTCTTCATG |
| | CCCAGTGAATGACTCACCTTGGCACAGACACAATGTTCGGGGTGGGCACAGT |
| | GCCTGCTTCCCGCCGCACCCCAGCCCCCCTCAAATGCCTTCCGAGAAGCCCA |
| | TTGAGCAGGGGGCTTGCATTGCACCCCAGCCTGACAGCCTGGCATCTTGGGA |
| | TAAAAGCAGCACAGCCCCCTAGGGGCTGCCCTTGCTGTGTGGCGCCACCGGC |
| | GGTGGAGAACAAGGCTCTATTCAGCCTGTGCCCAGGAAAGGGGATCAGGGGA |
| | TGCCCAGGCATGGACAGTGGGTGGCAGGGGGGGAGAGGAGGGCTGTCTGCT |
| | TCCCAGAAGTCCAAGGACACAAATGGGTGAGGGGAAGCGCGCAGAGTCTGCA |
| | TGCGTGAGGAAGCTCCTGGGCGCGTCACAGCCGCGCTATTCTCAGCGTCTCT |
| | CCTTTTATGGCTCCGGAAGTGAGCTGGGGTTGCTGGCAGCCTGGCTGGCACT |
| CRM_SP0028 | TCAGGGGTGCAGCTTTTTTTCTGTCTTTTACTCAGCCTGAGAAAGGTTGTCGTTTGACAAG |
| (SEQ ID NO: 17) | GTTTGTTCAGAGGTCGGATCTGAATCCAGCTCCAAGGCCCCAGCACCCAAGCCCTGACCC |
| | CATGGCTGCCTGCTGGCTGGGAGTGGCATTCTTTAGAACAGGAAAGGCAGTGAGTTCTC |
| | ATTGCATCAATACTTGCATTTGCTACAACAGAAGCTTTTTGTCTCTTCCTCACATTCCTTTAG |
| | AACACAAGCCTCCTTTTCTGCCTGTGGCCATCGGTCACTGGAGTCAGCCTCGTGAGTGGCT |
| | TGGTGGCGGATGGCACCGTCCTTTGTGGGAGAAAACAATGTTGCTGCCCAGGCCTTTCTG |
| | GAATGACCCCTTCCCACCCCCTGACCAGCCCCAGCAAGGCCCGGGGCTGGCTGCCTAGTT |
| | GATTGGAGCCAAGAGTTTGCTGAATGGATGAAGGGAGAAGGGACATCCAAGTGACTGA |
| | AGAAGATCTTAACAGAAAGCGCGCAGAGTCTGCATGCGTGAGGAAGCTCCTGGGCGCGT |
| | CACAGCCGCGCTATTCTCAGCGTCTCTCCTTTTATGGCTCCGGAAGTGAGCTGGGGTTGCT |
| | GGCAGCCTGGCTGGCACT |
| CRM_SP0029 | TCAGGGGTGCAGCTTTTTTTCTGTCTTTTACTCAGCCTGAGAAAGGTTGTCGTTTGACAAG |
| (SEQ ID NO: 18) | GTTTGTTCAGAGGTCGGATCTGAATCCAGCTCCAAGGCCCCAGCACCCAAGCCCTGACCC |
| | CATGGCTGCCTGCTGGCTGGGAGTGGCATTCTTTAGAACAGGAAAGGCAGTGAGTTCTC |
| | ATTGCATCAATACTTGCATTTGCTACAACAGAAGCTTTTTGTCTCTTCCTCACATTCCTTTAG |
| | AACACAAGCCTCCTTTTCTGCCTGTGGCCATCGGTCACTGGAGTCAGCCTCGTGAGTGGCT |
| | TGGTGGCGGATGGCACCGTCCTTTGTGGGAGAAAACAATGTTGCTGCCCAGGCCTTTCTG |
| | GAATGACCCCTTCCCACCCCCTGACCAGCCCCAGCAAGGCCCGGGGCTGGCTGCCTAGTT |
| | GATTGGAGCCAAGAGTTTGCTGAATGGATGAAGGGAGAAGGGACATCCAAGTGACTGA |
| | AGAAGATCTTAACAGAAAGCGCGCAGAGTCTGCATGCGTGAGGAAGCTCCTGGGCGCGT |
| | CACAGCCGCGCTATTCTCAGCGTCTCTCCTTTTATGGCTCCGGAAGTGAGCTGGGGTTGCT |
| | GGCAGCCTGGCTGGCACT |

TABLE 3
Cis-regulatory elements comprised in the promoters of Table 1

| Name | SEQUENCE |
|---|---|
| CRE0005_GFAP | AACATATCCTGGTGTGGAGTAGGGGACGCTGCTCTGACAGAGGCTCGGGGG |
| (SYNP_AST_ | CCTGAGCTGGCTCTGTGAGCTGGGGAGGAGGCAGACAGCCAGGCCTTGTCT |
| CRE0005_GFAP) | GCAAGCAGACCTGGCAGCATTGGGCTGGCCGCCCCCCAGGGCCTCCTCTTC |
| (SEQ ID NO: 19) | ATGCCCAGTGAATGACTCACCTTGGCACAGACACAATGTTCGGGGTGGGCA |
| | CAGTGCCTGCTTCCCGCCGCACCCCAGCCCCCCTCAAATGCCTTCCGAGAA |
| | GCCCATTGAGCAGGGGGCTTGCATTGCACCCCAGCCTGACAGCCTGGCATC |
| | TTGGGATAAAAGCAGCACAGCCCCCTAGGGGCTGCCCTTGCTGTGTGGCGC |
| | CACCGGCGGTGGAGAACAAGGCTCTATTCAGCCTGTGCCCAGGAAAGGGGA |
| | TCAGGGGATGCCCAGGCATGGACAGTGGGTGGCAGGGGGGGAGAGGAGG |
| | GCTGTCTGCTTCCCAGAAGTCCAAGGACACAAATGGGTGAGGGGA |
| CRE0007_GFAP | AACATATCCTGGTGTGGAGTAGGGGACGCTGCTCTGACAGAGGCTCGGGGG |
| (SYNP_AST_ | CCTGAGCTGGCTCTGTGAGCTGGGGAGGAGGCAGACAGCCAGGCCTTGTCT |
| CRE0007_GFAP) | GCAAGCAGACCTGGCAGCATTGGGCTGGCCGCCCCCCAGGGCCTCCTCTTC |
| (SEQ ID NO: 20) | ATGCCCAGTGAATGACTCACCTTGGCACAGACACAATGTTCGGGGTGGGCA |
| | CAGTGCCTGCTTCCCGCCGCACCCCAGCCCCCCTCAAATGCCTTCCGAGAA |
| | GCCCATTGAGCAGGGGGCTTGCATTGCACCCCAGCCTGACAGCCTGGCATC |
| | TTGGGATAAAAGCAGCAC |
| CRE0012_Arc | AGCGCGCAGAGTCTGCATGCGTGAGGAAGCTCCTGGGCGCGTCACAGCCG |
| (SYNP_NEU_ | CGCTATTCTCAGCGTCTCTCCTTTTATGGCTCCGGAAGTGAGCTGGGGTTGC |
| CRE0012_Arc) | TGGCAGCCTGGCTGGCACT |
| (SEQ ID NO: 21) | |
| CRE0001_S100B | CTTCCTCTTATATTTCACCAAGACTCAGTTCCTGAGCAAGAAACCACAGGCAC |
| (SYNP_AST_ | AGCAAGTGCCATGAAAAGCGGCTTTGTGTGGGGTGGGCTCTTCACACTCCAA |
| CRE0001_S100B) | TCTCCACTTCCTTCTCAAGGCCTCAAAAAAAGTTGAAAAATGAAAACAAAAGC |
| (SEQ ID NO: 22) | CCTGCTGTGTTGAGCTGGGCTCTGGCGTTGCCATGGACCCAGGGCAAACAG |
| | CGGTGCTCCTGCTCTGCCCCCGGCTCAGCTCATGCTGGGCCTGCACTTCTG |
| | GAAGGGAGCATGGACTTTGGAATGACTGGTTAGAACCCAAATGAATTAATGG |
| | AATTTGACATAGTTCAAAAATAATAAAATGTGATACCCATGAAATGCTGATATT |
| | CTGCCTTAATTTGCCAGATTGGGGGCC |
| CRE0009 S100B | TTCAGAGGTCGGATCTGAATCCAGCTCCAAGGCCCCAGCACCCAAGCCCTG |
| (SYNP_AST_ | ACCCCATGGCTGCCTGCTGGCTGGGAGTGGCATTCTTTAGAACAGGAAAGG |
| CRE0009_S100B) | CAGTGAGTTCTCATTGCATCAATACTTGCATTTGCTACAACAGAAGCTTTTTG |
| (SEQ ID NO: 23) | TCTCTTCCTCACATTCCTTTAGAACACAAGCCTCCTTTTCTGCCTGTGGCCAT |
| | CGGTCACTGGAGTCAGCCTCGTGAGTGGCTTGGTGGCGGATGGCACCGTCC |
| | TTTGTGGGAGAAAACAATGTTGCTGCCCAGGCCTTTCTGGAATGACCCCTTC |
| | CCACCCCCTGACCAGCCCCAGCAAGGCCCGGGGCTGGCTGCCTAGTTGATT |
| | GGAGCCAAGAGTTTGCTGAATGGATGAAGGGAGAAGGGACATCCAA |
| CRE0002 S100B | TCAGGGGTGCAGCTTTTTTTCTGTCTTTTACTCAGCCTGAGAAAGGTTGTCGT |
| (SYNP_AST_ | TTGACAAGGTTTGTTCAGAGGTCGGATCTGAATCCAGCTCCAAGGCCCCAGC |
| CRE0002_S100B) | ACCCAAGCCCTGACCCCATGGCTGCCTGCTGGCTGGGAGTGGCATTCTTTA |
| (SEQ ID NO: 24) | GAACAGGAAAGGCAGTGAGTTCTCATTGCATCAATACTTGCATTTGCTACAAC |
| | AGAAGCTTTTTGTCTCTTCCTCACATTCCTTTAGAACACAAGCCTCCTTTTCTG |
| | CCTGTGGCCATCGGTCACTGGAGTCAGCCTCGTGAGTGGCTTGGTGGCGGA |
| | TGGCACCGTCCTTTGTGGGAGAAAACAATGTTGCTGCCCAGGCCTTTCTGGA |
| | ATGACCCCTTCCCACCCCCTGACCAGCCCCAGCAAGGCCCGGGGCTGGCTG |
| | CCTAGTTGATTGGAGCCAAGAGTTTGCTGAATGGATGAAGGGAGAAGGGAC |
| | ATCCAAGTGACTGAAGAAGATCTTAACAGAA |
| CRE0006_AQP4 | CTAGCCCACAGGAAATGTCTGTCTATATCCAGGCAAGTACCTTGCTCATTGG |
| (SYNP_AST_ | ACCAACCCGAAACTGTTCAGGGAAGATCAGGGAAATCAACTCAGTTACAAAT |
| CRE0006_AQP4) | GGGATAATCATGCCCAGTAAAAACTACCTGTGGTGAATAAAGAGTTAACCCC |
| (SEQ ID NO: 37) | TGTTCCATCTTAGGTCACTATGCAGAGTACCAATGAGTACAAGAGATGGTGC |
| | CAAAGAGGGTGGCCCCTCCCTAGCTGGGAACAGTCAACCCTTAGGAACTAG |
| | ACTGTCAACACATCAGCCAGCCAGAGACAAGGGAAACCGTGGCAACCAAGT |
| | GTTGCTGGCACATTGTGAGGTGGTGATGGGAACTGCAGAGGCCCTGCACAG |
| | CATGCTAATGAGCCCAGGCAAACAT |
| CRE0008_AQP4 | TCCAAAGAAAAGCCAGATAAGTAGCTGATTATTGCATAGAGCTGACAGTATCA |
| (SYNP_AST_TO_ | CAGGAAGATCAGTAGTAGCAGCTCAAGTACAAAAAGGTTAATTAGCAATACTT |
| CRE0008v2_AQP4) | AATAAGAAAAACTACCTCTGGCAGGTGAAGAGTTAATCCCTGGTCAATTTTAA |
| (SEQ ID NO: 38) | GCTACTCTGCTGAGAGTACTAATAAGTGTAGGGGTTGGAGCCAATGAGGGTG |
| | ACCCCTTCCTTGATGGGAACAGTCATCCCTTAGGAACTGCCCTGGAAAGCAT |
| | CAGCCAGCCAGAAAACAGGGAAAGAGGCTGAGAAACCGTGGTAACCAAGTT |
| | TTGCTGGCACTTTGTAAAATGGTAACTGCAACTGCCGAGGCTGTGCAGAGAA |
| | TGCTAATAAGCCTAGGACAACCTGTAAAGAGTGGACCTAGAAAATGTCCACC |

TABLE 4
Minimal/Proximal Promoters comprised in the promoters of Table 1

| Name | SEQUENCE |
|---|---|
| CRE0003_CEND1_mp | GGGCTAGGCCTGCGCACCCACCCACCGACCCCTCACCCACCGACCCGTCACC |
| (SYNP_NEU_ | CACCGACCAAGGGGCACCCTGGCCTAGAGGGGATGCTGAGCGGGACCCGCC |
| CRE0003_CEND1_mp) | TCCTGCCTCTGGCAGTCCCAGATGGGACTTGGACCCCGCAGTTGCTCTCTCG |
| (SEQ ID NO: 25) | GACCCTAAGTTTCTACCCCTGGATCTAAGGCGGAGCTGGGTTTGCGGATCCCA |
| | CGGTTCCCGGCGGGGCGGGGCCCGGTCGCCCCTCCCCCTCCCCGCCCTCCT |
| | GCGCCGGGAGCAGTGCATTGTGGGAAACTCCCGA |
| CRE0009_SYN1_mp | TGCGGCGAGGCGCGTGCGCACTGCCAGCTTCAGCACCGCGGACAGTGCCTT |
| (SYNP_NEU_ | CGCCCCCGCCTGGCGGCGCGCGCCACCGCCGCCTCAGCACTGAAGGCGCGC |
| CRE0009_SYN1_mp) | TGACGTCACTCGCCGGTCCCCCGCAAACTCCCCTTCCCGGCCACCTTGGTCG |
| (SEQ ID NO: 26) | CGTCCGCGCCGCCGCCGGCCCAGCCGGACCGCACCACGCGAGGCGCGAGA |
| | TAGGGGGGCACGGGCGCGACCATCTGCGCTGCGGCGCCGGCGACTCAGCGC |
| | TGCCTCAGTCTGCGGTGGGCAGCGGAGGAGTCGTGTCGTGCCTGAGAGCGC |
| | AG |
| CRE0015_GAP43_mp | CGCTAGAGAGAGGGAGCGAGCATGTGCGATGAGCAATAGCTGTGGACCTTAC |
| (SYNP_NEU_ | AGTTGCTGCTAACTGCCCTGGTGTGTGTGAGGGAGAGAGAGGGAGGGAGGG |
| CRE0015_GAP43_mp) | AGAGAGAGCGCGCTAGCGCGAGAGAGCGAGTGAGCAAGCGAGCAGAAAAGA |
| (SEQ ID NO: 39) | GGTGGAGAGGGGGGGAATAAGAAAGAGAGAGAAGGAAAGGAGAGAAGGCAG |
| | GAAGAAGGCAAGGGACGAGACAA |
| CRE0016_Eno2_mp | TCGAGTTGGCTGGACAAGGTTATGAGCATCCGTGTACTTATGGGGTTGCCAGC |
| (SYNP_NEU_ | TTGGTCCTGGATCGCCCGGGCCCTTCCCCCACCCGTTCGGTTCCCCACCACC |
| CRE0016_Eno2_mp) | ACCCGCGCTCGTACGTGCGTCTCCGCCTGCAGCTCTTGACTCATCGGGGCCC |
| (SEQ ID NO: 40) | CCCGGGTCACATGCGCTCGCTCGGCTCTATAGGCGCCGCCCCCTGCCCACCC |
| | CCCGCCCGCGCTGGGAGCCGCAGCCGCCGCCACTCCTGCTCTCTCTGCGCC |
| | GCCGCCGTCACCACCGCCACCGCCACCGGCTGAGTCTGCAGTCCTCGAG |
| CRE0004_GFAP_mp | GAGCTCTCCCCATAGCTGGGCTGCGGCCCAACCCCACCCCCTCAGGCTATGC |
| (SYNP_AST_ | CAGGGGGTGTTGCCAGGGGCACCCGGGCATCGCCAGTCTAGCCCACTCCTTC |
| CRE0004_GFAP_mp) | ATAAAGCCCTCGCATCCCAGGAGCGAGCAGAGCCAGAGC |
| (SEQ ID NO: 41) | |

TABLE 5
synthetic promoter overview

| | Minimal/proximal | | |
|---|---|---|---|
| Promoter name | promoter | CRE | CRE |
| SP0013 | CRE0003_CEND1_mp | CRE0005_GFAP | |
| (SYNP_NA_SP0013) | | | |
| SP0014 | CRE0009_SYN1_mp | CRE0005_GFAP | |
| (SYNP_NA_SP0014) | | | |
| SP0026 | CRE0003_CEND1_mp | CRE0012_Arc | CRE0005_GFAP |
| SP0027 | CRE0009_SYN1_mp | CRE0012_Arc | CRE0005_GFAP |
| SP0030 | CRE0003_CEND1_mp | CRE0007_GFAP | |
| (SYNP_NA_SP0030) | | | |
| SP0031 | CRE0009_SYN1_mp | CRE0007_GFAP | |
| (SYNP_NA_SP0031) | | | |
| SP0032 | CRE0003_CEND1_mp | CRE0009_S100B | |
| (SYNP_NA_SP0032) | | | |
| SP0033 | CRE0009_SYN1_mp | CRE0009_S100B | |
| SP0019 | CRE0003_CEND1_mp | CRE0002_S100B | |
| (SYNP_NAO_SP0019) | | | |
| SP0020 | CRE0003_CEND1_mp | CRE0001_S100B | |
| (SYNP_NAO_SP0020) | | | |
| SP0021 | CRE0009_SYN1_mp | CRE0002_S100B | |
| (SYNP_NAO_SP0021) | | | |
| SP0022 | CRE0009_SYN1_mp | CRE0001_S100B | |
| (SYNP_NAO_SP0022) | | | |
| SP0028 | CRE0003_CEND1_mp | CRE0012_Arc | CRE0002_S100B |
| SP0029 | CRE0009_SYN1_mp | CRE0012_Arc | CRE0002_S100B |
| SP0011 | CRE004_GFAP_mp | CRE0006_AQP4 | |
| (SYNP_AST_SP0011) | | | |
| SP0034 | CRE0015_GAP43_mp | CRE0008_AQP4 | |
| (SYNP_NA_SP0034) | | | |
| SP0035 | CRE0015_GAP43_mp | CRE0006_AQP4 | |
| (SYNP_NA_SP0035) | | | |
| SP0036 | | | |
| (SYNP_NA_SP0036) | CRE0016_Eno2_mp | CRE0006_AQP4 | |

TABLE 6
Additional CREs and promoter elements

| Name | SEQUENCE |
|---|---|
| CRE0006_GFAP | AACATATCCTGGTGTGGAGTAGGGGACGCTGCTCTGACAGAGGCTCGGGGG |
| (SYNP_AST_ | CCTGAGCTGGCTCTGTGAGCTGGGGAGGAGGCAGACAGCCAGGCCTTGTCT |
| CRE0006_GFAP) | GCAAGCAGACCTGGCAGCATTGGGCTGGCCGCCCCCCAGGGCCTCCTCTTC |
| (SEQ ID NO: 27) | ATGCCCAGTGAATGACTCACCTTGGCACAGACACAATGTTCGGGGTGGGCA |
| | CAGTGCCTGCTTCCCGCCGCACCCCAGCCCCCCTCAAATGCCTTCCGAGAA |
| | GCCCATTGAGCAGGGGGCTTGCATTGCACCCCAGCCTGACAGCCTGGCATC |
| | TTGGGATAAAAGCAGCACAGCCCCCTAGGGGCTGCCCTTGCTGTGTGGCGC |
| | CA |
| CRE0008_GFAP | AACATATCCTGGTGTGGAGTAGGGGACGCTGCTCTGACAGAGGCTCGGGGG |
| (SYNP_AST_ | CCTGAGCTGGCTCTGTGAGCTGGGGAGGAGGCAGACAGCCAGGCCTTGTCT |
| CRE0008_GFAP) | GCAAGCAGACCTGGCAGCATTGGGCTGGCCGCCCCCCAGGGCCTCCTCTTC |
| (SEQ ID NO: 28) | ATGCCCAGTGAATGACTCACCTTGGCACAGACACAATGTTCGGGGTGGGCA |
| | CAGTGCCTGCTTCCCGCCGCACCCCAGCCCCCCTCAAATGCCTTCCGAGAA |
| | GCCCATTGAGCAGGGGGCTTGCATTGCACCCCAGCCTGACAGCCTGGCATC |
| | TTGGGATAAAAGCACCGGCGGTGGAGAACAAGGCTCTATTCAGCCTGTGCC |
| | CAGGAAAGGGGATCAGGGGATGCCCAGGCATGGACAGTGGGTGGCAGGGG |
| | GGGAGAGGAGGGCTGTCTGCTTCCCAGAAGTCCAAGGACACAAATGGGTGA |
| | GGGGA |
| CRE0001_CEND1_mp | GATCCCACGGTTCCCGGCGGGGCGGGGCCCGGTCGCCCCTCCCCCTCCCC |
| (SYNP_NEU_ | GCCCTCCTGCGCCGGGAGCAGTGCATTGTGGGAAACTC |
| CRE0001_CEND1_mp1 | |
| (SEQ ID NO: 29) | |
| CRE0002_CEND1_mp | GGGCTAGGCCTGCGCACCCACCCACCGACCCCTCACCCACCGACCCGTCAC |
| (SYNP_NEU_ | CCACCGACCAAGGGGCACCCTGGCCTAGAGGGGATGCTGAGCGGGACCCG |
| CRE0002_CEND1_mp) | CCTCCTGCCTCTGGCAGTCCCAGATGGGACTTGGACCCCGCAGTTGCTCTC |
| (SEQ ID NO: 30) | TCGGACCCTAAGTTTCTACCCCTGGATCTAAGGCGGAGCTGGGTTTGCGGAT |
| | CCCACGGTTCCCGGCGGGGCGGGGCCCGGTCGCCCCTCCCCCTCCCCGCC |
| | CTCCTGCGCCGGGAGCAGTGCATTGTGGGAAACTCCCGAGCTCTCCTCCGC |
| | GTTCGCAGCCGCCGTCATCCCGCGGAGGAGCGCGCAGCCCC |
| CRE0008_SYN1_mp | TGCAGAGGGCCCTGCGTATGAGTGCAAGTGGGTTTTAGGACCAGGATGAGG |
| (SYNP_NEU_ | CGGGGTGGGGGTGCCTACCTGACGACCGACCCCGACCCACTGGACAAGCA |
| CRE0008_SYN1_mp) | CCCAACCCCCATTCCCCAAATTGCGCATCCCCTATCAGAGAGGGGGAGGGG |
| (SEQ ID NO: 31) | AAACAGGATGCGGCGAGGCGCGTGCGCACTGCCAGCTTCAGCACCGCGGA |
| | CAGTGCCTTCGCCCCCGCCTGGCGGCGCGCGCCACCGCCGCCTCAGCACT |
| | GAAGGCGCGCTGACGTCACTCGCCGGTCCCCCGCAAACTCCCCTTCCCGGC |
| | CACCTTGGTCGCGTCCGCGCCGCCGCCGGCCCAGCCGGACCGCACCACGC |
| | GAGGCGCGAGATAGGGGGGCACGGGCGCGACCATCTGCGCTGCGGCGCC |
| | GGCGACTCAGCGCTGCCTCAGTCTGCGGTGGGCAGCGGAGGAGTCGTGTC |
| | GTGCCTGAGAGCGCAG |
| CRE0013_SYN1_mp | TGCGGCGAGGCGCGTGCGCACTGCCAGCTTCAGCACCGCGGACAGTGCCT |
| (SYNP_NEU_ | TCGCCCCCGCCTGGCGGCGCGCGCCACCGCCGCCTCAGCACTGAAGGCGC |
| CRE0013_SYN1_mp) | GCTGACGTCACTCGCCGGTCCCCCGCAAACTCCCCTTCCCGGCCACCTTGG |
| (SEQ ID NO: 32) | TCGCGTCCGCGCCGCCGCCGGCCCAGCCGGACCGCACCACGCGAGGCGC |
| | GAGATAGGGGGGCACGGGCGCGACCATCTGCGCTGCGGCGCCGGCGACTC |
| | AGCGCTGCCTCAGTCT |

### EXAMPLE 2

The synthetic NS-specific promoters according to the present invention were designed through reviewing scientific literature to identify genes and their respective promoters which are highly active in NS cells.

During the design of these promoters, particular shortcomings of known NS-specific promoters were considered. First of all, known NS-specific promoters which are specific for a NS cell type (e.g. Synapsin-1, CAMKIIa and GFAP) are not expressed in the whole cellular population (e.g. not expressed in all neurones/astrocytes). This has been shown for GFAP by (Zhang et al., 2019) and can be seen from distribution of Syn-1 in neurones from the Allen brain atlas. Secondly, the majority of the known CREs, promoter elements and promoters are too large to be included in a self-complementary AAV vector (scAAV) (depending on the size of the transgene, the size of the promoter may need to be less than 1000 bp, preferably less than 900 bp, more preferably less than 800 bp, most preferably less than 700 bp). Additionally, expression may be required in a specific cell type or a combination of cell types across the entire NS, suitably the entire CNS or the entire brain.

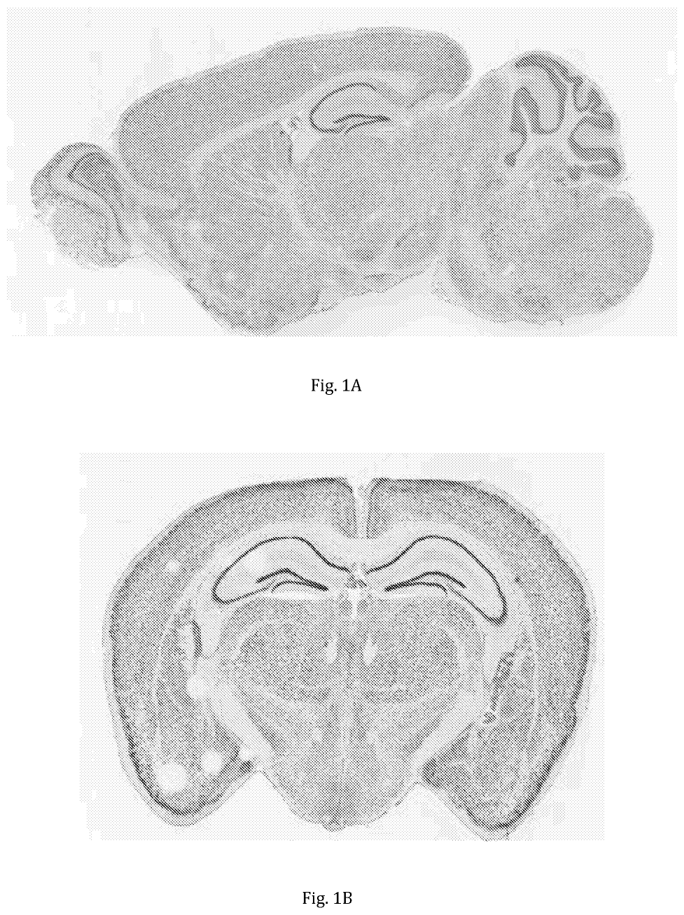

Currently known promoters are not able to address these shortcomings and there is a need in gene therapy to develop short, cell-type NS-specific promoters both with targeted localised expression and also with expression across the entire NS. For example, the requirement for an expression across the entire NS (e.g. the entire brain) is highlighted by the expression pattern of the HTT (huntingtin) and CYP46A1 genes in the adult mouse brain shown in FIG. 1A and FIG. 1B. Since the HTT (huntingtin) gene is expressed throughout the brain, it may be beneficial for any potential expression product suppressing the faulty huntingtin gene and/or counteracting or alleviating the detrimental effects of the faulty huntingtin to be expressed throughout the brain. Similarly, since the CYP46A1 gene is expressed throughout the brain, it may be beneficial for any potential supplementary CYP46A1 expression to be throughout the brain.

Gene expression in all neurones as well as astrocytes and/or oligodendrocytes across the CNS may be desirable in treatment of some diseases such as Huntington's disease. Expression in astrocytes and oligodendrocytes may be beneficial as glial cells are implicated in Huntington's disease (Shin et al., 2005). Additionally, in a therapeutic setting, for AAV-RNAi mediated HTT lowering therapeutics to rescue of behavioural phenotypes in the YAC128 mouse model, it was found that astrocytic transduction is required (Stanek, Bu and Shihabuddin, 2019).

Therefore, the present invention sets out to design tandem NS promoters which are active in multiple NS cell types while addressing some of the shortcomings listed above. For example, the promoter design involved combination of one or more CRE together with a promoter element in order to broaden the cell tropism compared to the individual CRE/promoter element in order to create promoters active in multiple NS cell types and also to address the drawback of known promoters not being expressed in the whole cellular population. Additionally, in order to address the drawback of known CREs, promoter elements and promoters being too large to be included in an AAV vector such as self-complementary AAV vector (scAAV), some of the CREs and promoter elements of the present invention have been shortened using bioinformatic analysis, literature searching and publicly available genomic databases but are still expected to be active CREs and promoter elements. For example, CRE0006_GFAP (SEQ ID NO: 27), CRE0008_GFAP (SEQ ID NO: 28) and CRE0007_GFAP (SEQ ID NO: 20) have been shortened but are predicted to be active regulatory sequences conferring astrocyte-specific activity when operably linked to a promoter element. Similarly, CRE0001_S100B (SEQ ID NO: 22), CRE0009_S100B (SEQ ID NO: 23) and CRE0002_S100B (SEQ ID NO: 24) have been shortened but are predicted to be active regulatory sequences conferring astrocyte-specific and oligodendrocyte-specific activity when operably linked to a promoter element. The NS-specific CRE according to the present invention is preferably shortened, i.e. CRE0006_GFAP (SEQ ID NO: 27), CRE0008_GFAP (SEQ ID NO: 28), CRE0007_GFAP (SEQ ID NO: 20), CRE0001_S100B (SEQ ID NO: 22), CRE0009_S100B (SEQ ID NO: 23) and CRE0002_S100B (SEQ ID NO: 24). These are particularly preferred CREs due to their short size and predicted NS-specific activity. Additionally, CRE0003_CEND1_mp (SEQ ID NO: 25) has been shortened but is predicted to be an active regulatory sequence in neuronal cells. Finally, CRE0009_SYN1_mp (SEQ ID NO: 26) and CRE0013_SYN1_mp (SEQ ID NO: 32) have been shortened but are predicted to be active regulatory sequences in neuronal cells. The promoter element according to the present invention is preferably shortened, i.e. CRE0003_CEND1_mp (SEQ ID NO: 25), CRE0009_SYN1_mp (SEQ ID NO: 26) and CRE0013_SYN1_mp (SEQ ID NO: 32) are particularly preferred promoter elements due to their short size and predicted neuronal activity.

The synthetic NS-specific promoters according to the present invention are operably linked to a nucleic acid sequence encoding the CYP46A1 transgene and a Human influenza hemagglutinin (HA) tag and experimentally tested in wildtype C57BL6/J mice. The synthetic NS-specific promoters according to the present invention operably linked to a nucleic acid sequence encoding the CYP46A1 transgene and a HA tag are administered intravenously in a viral vector. Vector copy number will be assessed in brain and spinal cord tissue sections by qPCR analysis of the viral transgene CYP46A1 normalised to internal genomic DNA copy number control to confirm equivalent injected doses. Western blot will be performed to assess the protein expression of the HA tagged transgene in the brain and spinal cord tissue. Finally, immunofluorescent staining will be performed on brain and spinal cord tissue sections to assess the expression of the transgene within CNS cell types. Similarly, immunofluorescent staining can be performed on PNS tissue sections to assess the expression of the transgene within PNS cell types. Specifically, double staining will be performed using the HA tag to mark CYP46A1 gene expression and standard markers for neurones, astrocytes, oligodendrocytes and microglia.

SP0013 (SEQ ID NO: 1) is predicted to be active in neurones and astrocytes. SP0014 (SEQ ID NO: 2) is predicted to be active in neurones and astrocytes. SP0026 (SEQ ID NO: 3) is predicted to be active in excitatory neurones and astrocytes. SP0027 (SEQ ID NO: 4) is predicted to be active in excitatory neurones and astrocytes. SP0030 (SEQ ID NO: 5) is predicted to be active in neurones and astrocytes. SP0031 (SEQ ID NO: 6) is predicted to be active in neurones and astrocytes. SP0032 (SEQ ID NO: 7) is predicted to be active in neurones, astrocytes and oligodendrocytes. SP0033 (SEQ ID NO: 8) is predicted to be active in neurones, astrocytes and oligodendrocytes. SP0019 (SEQ ID NO: 9) is predicted to be active in neurones, astrocytes and oligodendrocytes. SP0020 (SEQ ID NO: 10) is predicted to be active in neurones, astrocytes and oligodendrocytes. SP0021 (SEQ ID NO: 11) is predicted to be active in neurones, astrocytes and oligodendrocytes. SP0022 (SEQ ID NO: 12) is predicted to be active in neurones, astrocytes and oligodendrocytes. SP0028 (SEQ ID NO: 13) is predicted to be active in excitatory neurones, astrocytes and oligodendrocytes. SP0029 (SEQ ID NO: 14) is predicted to be active in excitatory neurones, astrocytes and oligodendrocytes. SP0011 (SEQ ID NO: 33) is predicted to be active in neurones and astrocytes. SP0034 (SEQ ID NO: 34) is predicted to be active in neurones and astrocytes. SP0035 (SEQ ID NO: 35) is predicted to be active in neurones and astrocytes. SP0036 (SEQ ID NO: 36) is predicted to be active in neurones and astrocytes.

Bioinformatic analysis of RNA sequencing data predicts that some of the genes associated with the CREs and/or promoter elements of the present invention (agp4, cend1, eno2, gfap, s100B, syn1) are expressed in the dorsal root ganglion and tibial nerve. Therefore, CREs and/or promoter elements associated with these genes are predicted to be expressed in cells of the PNS. CRE0001_S100B (SEQ ID NO: 22), CRE0002_S100B (SEQ ID NO: 24), CRE0005_GFAP (SEQ ID NO: 19), CRE0007_GFAP (SEQ ID NO: 20), CRE0009_S100B (SEQ ID NO: 23), CRE0006_GFAP (SEQ ID NO: 27), CRE0008_GFAP (SEQ ID NO: 28), CRE0006_AQP4 (SEQ ID NO: 37), CRE0008_AQP4 (SEQ ID NO: 38), CRE0003_CEND1_mp (SEQ ID NO: 25), CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0013_SYN1_mp (SEQ ID NO: 32), CRE0016_Eno2_mp (SEQ ID NO: 40) and CRE0004_GFAP_mp (SEQ ID NO: 41), or functional variants thereof are predicted to be active in cells of the PNS. Bioinformatic analysis of single cell RNA sequencing data predicts that some of the genes associated with the CREs and/or promoter elements of the present invention (agp4, cend1, eno2, gfap, s100B, syn1) are expressed in sensory neurones, PNS sympathetic neurones and PNS enteric neurones. Therefore, CREs and/or promoter elements associated with these genes are predicted to be expressed in sensory neurones, PNS sympathetic neurones and PNS enteric neurones. CRE0001_S100B (SEQ ID NO: 22), CRE0002_S100B (SEQ ID NO: 24), CRE0005_GFAP (SEQ ID NO: 19), CRE0007_GFAP (SEQ ID NO: 20), CRE0009_S100B (SEQ ID NO: 23), CRE0006_GFAP (SEQ ID NO: 27), CRE0008_GFAP (SEQ ID NO: 28), CRE0006_AQP4 (SEQ ID NO: 37), CRE0008_AQP4 (SEQ ID NO: 38), CRE0003_CEND1_mp (SEQ ID NO: 25), CRE0009_SYN1_mp (SEQ ID NO: 26), CRE0001_CEND1_mp (SEQ ID NO: 29), CRE0002_CEND1_mp (SEQ ID NO: 30), CRE0008_SYN1_mp (SEQ ID NO: 31), CRE0013_SYN1_mp (SEQ ID NO: 32), CRE0016_Eno2_mp (SEQ ID NO: 40) and CRE0004_GFAP_mp (SEQ ID NO: 41), or functional variants thereof are predicted to be active in sensory neurones, PNS sympathetic neurones and/or PNS enteric neurones.

### EXAMPLE 3

In order to assess the activity in the NS of a CRE, CRM, a promoter element or a synthetic NS-specific promoter according to this invention, it is desirable to operably link the synthetic NS-specific promoter or a synthetic NS-specific promoter comprising the CRE, CRM, a promoter element according to the present invention to a reporter gene (such as GFP). The expression construct comprising the above is administered intravenously in a viral vector penetrating the CNS in an animal (such as a mouse or a rat). Following a time period (such as 1 week, 2 weeks, 3 weeks, 1 month, 2 months, 3 months), the animal is sacrificed. The CNS and PNS of the animal is processed into tissue sections and immunostaining (fluorescent and colourimetric) may be performed to assess the expression of the reporter throughout the CNS and PNS and in different CNS and PNS cell types. If quantification is required, a western blot for the reporter is performed on CNS and PNS tissue. Performing these techniques is routine and known.

### EXAMPLE 4

Described herein in is a method of manufacturing viral vectors from Pro10/HEK293 cells that have been engineered to stably integrate the CYP46A1 gene.

The stable cell line, Pro10/HEK293, as described in U.S. Pat. No. 9,441,206, is ideal for scalable production of AAV vectors. This cell line can be contacted with an expression vector comprising CYP46A1 gene operatively linked to a NS-specific promoter according to any one of SEQ ID NO: 1-14, 33-36 or variants thereof. Clonal populations having CYP46A1 integrated into their genome are selected using methods well known in the art.

Pro10/HEK293 cells stably encompassing CYP46A1 gene are transfected with a Packaging plasmid encoding Rep2 and serotype-specific Cap2: AAV-Rep/Cap, and the Ad-Helper plasmid (XX680: encoding adenoviral helper sequences).

Transfection. On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The cocktail is inverted to mix prior to being incubated at room temperature. The transfection cocktail is pipetted into the flasks and placed back in the shaker/incubator. All optimization studies are carried out at 30 mL culture volumes followed by validation at larger culture volumes. Cells are harvested 48 hours post-transfection.

Production of rAAV Using Wave Bioreactor Systems. Wave bags are seeded 2 days prior to 30 transfection. Two days post-seeding the wave bag, cell culture counts are taken and the cell culture is then expanded/diluted before transfection. The wave bioreactor cell culture is then transfected. Cell culture is harvested from the wave bio-reactor bag at least 48 hours post-transfection.

Titer: AAV titers are calculated after DNase digestion using qPCR against a standard curve (AAV ITR specific) and primers specific to CYP46A1 gene.

Harvesting Suspension Cells from Shaker Flasks and 60 Wave Bioreactor Bags. 48 hours post-transfection, cell cultures are collected into 500 mL polypropylene conical tubes (Corning) either by pouring from shaker flasks or pumping from wave bioreactor bags. The cell culture is then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 min. At this point, the pellet can either be stored in NLT-60° C. or continued through purification.

Titering rAAV from Cell Lysate Using qPCR. 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl2, 5 mM MgCl2, 50 mM Tris-HCl pH 8.0) followed by sonication to lyse the cells efficiently. 300 μL is then removed and placed into a 1.5 mL microfuge tube. 140 units of DNase I is then added to each sample and incubated at 37° C. for 1 hour. To determine the effectiveness of the DNase digestion, 4-5 mg of CYP46A1 plasmid is spiked into a non-transfected cell lysate with and without the addition of DNase. 50 μL of EDTA/Sarkosyl solution (6.3% sarkosyl, 62.5 mM EDTA pH 8.0) is added to each tube and incubated at 70° C. for 20 minutes. 50 μL of Proteinase K (10 mg/mL) is then added and incubated at 55° C. for at least 2 hours. Samples are boiled for 15 minutes to inactivate the Proteinase K. An aliquot is removed from each sample to be analyzed by qPCR. Two qPCR reactions are carried out in order to effectively determine how much rAAV vector is generated per cell. One qPCR reaction is set up using a set of primers designed to bind to a homologous sequence on the backbones of plasmids XX680, pXR2 and CYP46A1. The second qPCR reaction is set up using a set of primers to bind and amplify a region within the CYP46A1 gene. qPCR is conducted using Sybr green reagents and Light cycler 480 from Roche. Samples are denatured at 95° C. for 10 minutes followed by 45 cycles (90° C. for 10 sec, 62° C. for 10 sec and 72° C. for 10 sec) and melting curve (1 cycle 99° C. for 30 sec, 65° C. for 1 minute continuous).

Purification of rAAV from Crude Lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art can use as mechanical methods such as microfluidization or chemical methods such as detergents, etc., followed by a clarification step using depth filtration or Tangential Flow Filtration (TFF).

AAV Vector Purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc.), which are incorporated herein by reference in their entireties.

### EXAMPLE 5—IN VITRO TESTING OF PROMOTERS

A selection of the NS-specific promoters according to the present invention were tested in neuroblastoma-derived SH-SY5Y cells.

### Materials and Methods

### Cell Maintenance and Transfection

SH-SY5Y cells were cultured in HAM F12 media with 1 mM L-Glutamine (Gibco 11765-054), 15% heat-inactivated FBS (ThermoFisher 10500064), 1% non-essential amino acids (Merck M1745-100ML), and 1% penicillin/streptomycin (ThermoFisher 15140122). The cells were passaged twice a week between 1:3 and 1:4 to maintain a healthy cell density of between 70-80%. The cells were kept under passage number 20. For transfections, the cells were seeded at 10⁵ cells/well into an adherent 48 well plate. 24 hours post-seeding, 300 ng plasmid was transfected into the cells using Lipofectamine3000 reagent (ThermoFisher L3000008).

The plasmid which was transfected into the SHSY5Y cell line comprises SP0013, SP0014, SP0030, SP0031, SP0032, SP0019, SP0020, SP0021, SP0033, SP0011, SP0034, SP0035 or SP0036 operably linked to GFP.

### Flow Cytometry

48 hours after transfection, SH-SY5Y cells were washed with PBS before dissociation with 0.05% trypsin. The cells were collected and resuspended in 90% PBS, 10% FBS solution. The GFP expression of the cells was assessed by flow cytometry using the Attune Nxt Acoustic Focusing Cytometer. The cell viability dye 7-AAD (ThermoFisher 00-6993-50) was mixed with the control cell population to identify and exclude the dead cells. The expression of GFP was measured in the living, single cell population using a blue 488 nm laser at the band-pass filter 510/10 nm. Untransfected cells were used to set the gates for GFP-negative and GFP-positive cells. The number of GFP-positive single cells and the median GFP fluorescence of all GFP-positive cells was calculated by the Attune Nxt Software.

### Results

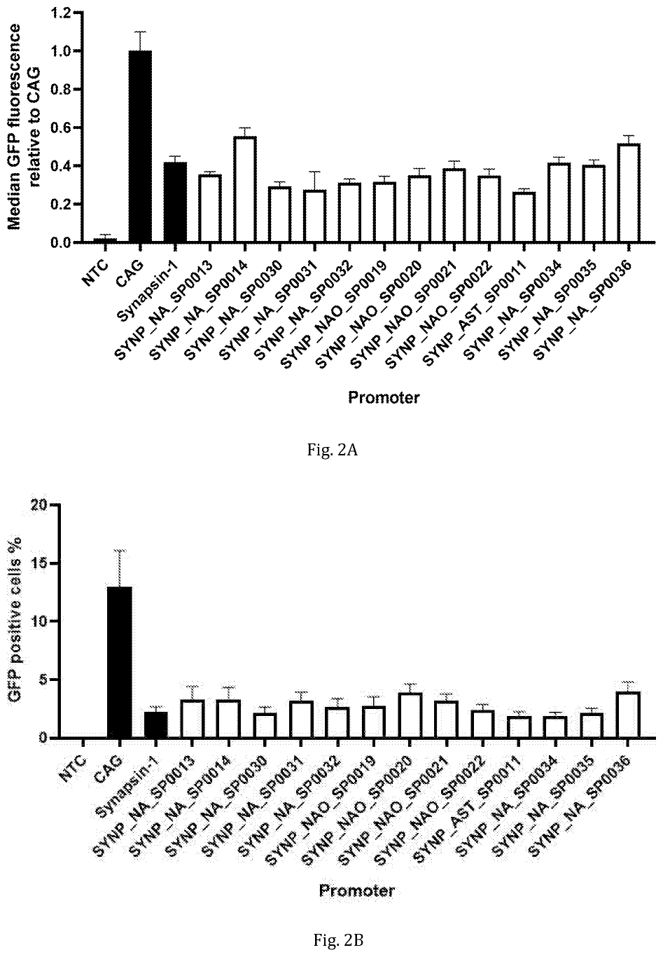

The results of this experiment are shown in FIGS. 2A and 2B. Neuroblastoma-derived SH-SY5Y cells transfected with expression cassette comprising SP0013, SP0014, SP0030, SP0031, SP0032, SP0019, SP0020, SP0021, SP0022, SP0011, SP0034, SP0035 or SP0036 operably linked to GFP were assessed for median GFP expression and percentage of GFP positive cells by flow cytometry. Expression cassettes comprising known promoters Synapsin-1 and CAG operably linked to GFP were used as controls. All tested promoters have comparable transfection efficiency and median GFP expression to the neuronal-specific control promoter Synapsin-1 (see FIGS. 2A and 2B). Control promoter CAG showed 2 to 3 times higher transfection efficiency (FIG. 2B) and around 2.5 higher median GFP expression compared to control promoter Synapsin-1 and the tested synthetic NS-specific promoters (FIG. 2A).

Synthetic NS-specific promoter SP0028 (SEQ ID NO: 13) is a similar design to synthetic NS-specific promoter SP0019 (SEQ ID NO: 9) as both comprise CRE0003_CEND1_mp (SEQ ID NO: 25) and CRE0002_S100B (SEQ ID NO: 24). In addition to these elements, SP0028 (SEQ ID NO: 13) further comprises CRE0012_Arc (SEQ ID NO: 21). As such, SP0028 (SEQ ID NO: 13) may be expected to perform similarly to SP0019 (SEQ ID NO: 9).

Synthetic NS-specific promoter SP0029 (SEQ ID NO: 14) is a similar design to synthetic NS-specific promoter SP0021 (SEQ ID NO: 11) as both comprise CRE0009_SYN1_mp (SEQ ID NO: 26) an CRE0002_S100B (SEQ ID NO: 24). In addition to these elements, SP0029 (SEQ ID NO: 14) further comprises CRE0012_Arc (SEQ ID NO: 21). As such, SP0029 (SEQ ID NO: 14) may be expected to perform similarly to SP0021 (SEQ ID NO: 11).

Synthetic NS-specific promoter SP0026 (SEQ ID NO: 3) is a similar design to synthetic NS-specific promoter SP0013 (SEQ ID NO: 1) as both comprise CRE0003_CEND1_mp (SEQ ID NO: 25) and CRE0005_GFAP (SEQ ID NO: 19). In addition to these elements, SP0026 (SEQ ID NO: 3) further comprises CRE0012_Arc (SEQ ID NO: 21). As such, SP0026 (SEQ ID NO: 3) may be expected to perform similarly to SP0013 (SEQ ID NO: 1).

Synthetic NS-specific promoter SP0027 (SEQ ID NO: 4) is a similar design to synthetic NS-specific promoter SP0014 (SEQ ID NO: 2) as both comprise CRE0009_SYN1_mp (SEQ ID NO: 26) and CRE0005_GFAP (SEQ ID NO: 19). In addition to these elements, SP0027 (SEQ ID NO: 4) further comprises CRE0012_Arc (SEQ ID NO: 21). As such, SP0027 (SEQ ID NO: 4) may be expected to perform similarly to SP0014 (SEQ ID NO: 2).

Synthetic NS-specific promoter SP0033 (SEQ ID NO: 8) is a similar design to SP0021 (SEQ ID NO: 11) as both comprise CRE0009_SYN1_mp (SEQ ID NO: 26). SP0033 (SEQ ID NO: 8) comprises CRE0009_S100B (SEQ ID NO: 23) which is a shorter version of CRE0002_S100B (SEQ ID NO: 24) which is in turn comprised in SP0021 (SEQ ID NO: 11). Therefore, SP0033 (SEQ ID NO: 8) is a shorter version of SP0021 (SEQ ID NO: 11) and, as such, may be expected to perform similarly.

### BIBLIOGRAPHY

Boussicault, L. et al. (2016) ‘CYP46A1, the rate-limiting enzyme for cholesterol degradation, is neuroprotective in Huntington's disease’, Brain, 139(3), pp. 953-970. doi: 10.1093/brain/awv384.
Djelti, F. et al. (2015) ‘CYP46A1 inhibition, brain cholesterol accumulation and neurodegeneration pave the way for Alzheimer's disease’, Brain, 138(8), pp. 2383-2398. doi: 10.1093/brain/awv166.
Hammond, S. L. et al. (2017) ‘Cellular selectivity of AAV serotypes for gene delivery in neurons and astrocytes by neonatal intracerebroventricular injection’, PLoS ONE, 12(12), pp. 1-22. doi: 10.1371/journal.pone.0188830.
Jakobsson, J. and Lundberg, C. (2006) ‘Lentiviral vectors for use in the central nervous system’, Molecular Therapy. The American Society of Gene Therapy, 13(3), pp. 484-493. doi: 10.1016/j.ymthe.2005.11.012.
Kacher, R. et al. (2019) ‘CYP46A1 gene therapy deciphers the role of brain cholesterol metabolism in Huntington's disease’, Brain: a journal of neurology, 142(8), pp. 2432-2450. doi: 10.1093/brain/awz174.
Schulte, J. and Littleton, J. T. (2011) ‘The biological function of the Huntingtin protein and its relevance to Huntington's Disease pathology.’, Current trends in neurology, 5, pp. 65-78. Available at: http://www.ncbi.nlm.nih.gov/pubmed/22180703% 0Ahttp://www.pubmedcentral.nih.gov/article render.fcgi?artid=PMC3237673.
Shin, J. et al. (2005) ‘Expression of mutant huntingtin in glial cells contributes to neuronal excitotoxicity’, 171(6), pp. 1001-1012. doi: 10.1083/jcb.200508072.
Stanek, L. M., Bu, J. and Shihabuddin, L. S. (2019) ‘Neurobiology of Disease Astrocyte transduction is required for rescue of behavioral phenotypes in the YAC128 mouse model with AAV-RNAi mediated HTT lowering therapeutics’, Neurobiology of Disease. Elsevier, 129 (November 2018), pp. 29-37. doi: 10.1016/j.nbd.2019.04.015.
Tanguy, Y. et al. (2015) ‘Systemic AAVrh10 provides higher transgene expression than AAV9 in the brain and the spinal cord of neonatal mice’, Frontiers in Molecular Neuroscience, 8 (JULY), pp. 1-10. doi: 10.3389/fnmol.2015.00036.
Zhang, Z. et al. (2019) ‘The Appropriate Marker for Astrocytes: Comparing the Distribution and Expression of Three Astrocytic Markers in Different Mouse Cerebral Regions’, BioMed Research International, 2019. doi: 10.1155/2019/9605265.

1. A synthetic nervous system (NS)-specific promoter comprising or consisting of at least one of the following cis-regulatory elements (CREs) or functional variants thereof:
CRE0001_S100B (SEQ ID NO: 22) or a functional variant thereof;
CRE0002_S100B (SEQ ID NO: 24) or a functional variant thereof;
CRE0005_GFAP (SEQ ID NO: 19) or a functional variant thereof;
CRE0007_GFAP (SEQ ID NO: 20) or a functional variant thereof;
CRE0009_S100B (SEQ ID NO: 23) or a functional variant thereof;
CRE0012_Arc (SEQ ID NO: 21) or a functional variant thereof;
CRE0006_GFAP (SEQ ID NO: 27) or functional variant thereof;
CRE0008_GFAP (SEQ ID NO: 28) or functional variant thereof;
CRE0006_AQP4 (SEQ ID NO: 37) or functional variant thereof; and
CRE0008_AQP4 (SEQ ID NO: 38) or functional variant thereof
operably linked to a promoter element selected from:
CRE0003_CEND1_mp (SEQ ID NO: 25) or a functional variant thereof;
CRE0009_SYN1_mp (SEQ ID NO: 26) or a functional variant thereof,
CRE0001_CEND1_mp (SEQ ID NO: 29) or a functional variant thereof;
CRE0002_CEND1_mp (SEQ ID NO: 30) or a functional variant thereof;
CRE0008_SYN1_mp (SEQ ID NO: 31) or a functional variant thereof;
CRE0013_SYN1_mp (SEQ ID NO: 32) or a functional variant thereof
CRE0015_GAP43_mp (SEQ ID NO: 39) or a functional variant thereof;
CRE0016_Eno2_mp (SEQ ID NO: 40) or a functional variant thereof; and/or
CRE0004_GFAP_mp (SEQ ID NO: 41) or a functional variant thereof.

2. A synthetic NS-specific promoter according to claim 1 comprising or consisting of at least one of the following cis-regulatory elements (CREs) or functional variants thereof:
CRE0001_S100B (SEQ ID NO: 22) or a functional variant thereof;
CRE0007_GFAP (SEQ ID NO: 20) or a functional variant thereof;
CRE0009_S100B (SEQ ID NO: 23) or a functional variant thereof;
CRE0006_AQP4 (SEQ ID NO: 37) or functional variant thereof; and
CRE0008_AQP4 (SEQ ID NO: 38) or functional variant thereof
operably linked to a promoter element selected from:
CRE0001_CEND1 mp (SEQ ID NO: 29) or a functional variant thereof;
CRE0013_SYN1_mp (SEQ ID NO: 32) or a functional variant thereof;
CRE0015_GAP43_mp (SEQ ID NO: 39) or a functional variant thereof;
CRE0016_Eno2_mp (SEQ ID NO: 40) or a functional variant thereof; and/or
CRE0004_GFAP_mp (SEQ ID NO: 41) or a functional variant thereof.

3. A synthetic NS-specific promoter according to claim 1 or 2 comprising or consisting of at least one of the following CREs or functional variants thereof:
CRE0001_S100B (SEQ ID NO: 22) or a functional variant thereof;
CRE0007_GFAP (SEQ ID NO: 20) or a functional variant thereof;
CRE0009_S100B (SEQ ID NO: 23) or a functional variant thereof;
CRE0006_AQP4 (SEQ ID NO: 37) or functional variant thereof; and
CRE0008_AQP4 (SEQ ID NO: 38) or functional variant thereof
operably linked to a promoter element selected from:
CRE0003_CEND1_mp (SEQ ID NO: 25) or a functional variant thereof;
CRE0009_SYN1_mp (SEQ ID NO: 26) or a functional variant thereof;
CRE0015_GAP43_mp (SEQ ID NO: 39) or a functional variant thereof;
CRE0016_Eno2_mp (SEQ ID NO: 40) or a functional variant thereof; and/or
CRE0004_GFAP_mp (SEQ ID NO: 41) or a functional variant thereof.

4. The synthetic NS-specific promoter according to claim 3 wherein the functional variant comprises a sequence which is at least 60%, 65%, 70%, 75%, 80%, 90%, 95%, 96%, 97%, 98% or 99% identical to any one of SEQ ID NOs 20, 22-23, 25-26, 37-41.

5. The synthetic NS-specific promoter according to claims 1-3 comprising or consisting of a sequence according to any one of SEQ ID NOs 1-14, 33-36, or a functional variant thereof.

6. The synthetic NS-specific promoter according to claim 5 comprising a sequence which is at least 60%, 65%, 70%, 75%, 80%, 90%, 95%, 96%, 97%, 98% or 99% identical to any one of SEQ ID NOs 1-14, 33-36.

7. A NS-specific cis-regulatory element (CRE) comprising a sequence according to any one of SEQ ID NOs: 19-24, 27, 28, 37, 38, or a functional variant thereof.

8. The NS-specific cis-regulatory elements (CRE) of claim 7 comprising a sequence which is at least 60%, 65%, 70%, 75%, 80%, 85%, 90%, 95%, 96%, 97%, 98% or 99% identical to any one of SEQ ID NOs: 19-24, 27, 28, 37, 38.

9. A synthetic NS-specific cis-regulatory module (CRM) comprising a sequence according to any one of SEQ ID NO: 15-18 or comprising a CRE according claim 7 or 8.

10. A synthetic NS-specific promoter comprising a CRE according to claims 7 or 8 or a CRM according to claim 9.

11. An isolated promoter element comprising a sequence according to any one of SEQ ID NOs: 25-26, 29-32, 39-41, or a functional variant thereof.

12. The isolated promoter element according to claim 11 comprising a sequence which is at least 60%, 65%, 70%, 75%, 80%, 85%, 90%, 95%, 96%, 97%, 98% or 99% identical to any one of SEQ ID NOs: 25-26, 29-32, 39-41.

13. A synthetic NS-specific promoter comprising a promoter element according to claim 11 or 12.

14. An expression cassette comprising a synthetic NS-specific promoter according to any one of claims 1-6, 10 or 13 operably linked to a nucleic acid sequence encoding an expression product.

15. A vector comprising a synthetic NS-specific promoter according to any one of claims 1-6, 10 or 13 or an expression cassette according to claim 14.

16. The vector of claim 15 which is a viral vector, e.g. an AAV vector, an adenoviral vector, a retroviral vector or a lentiviral vector.

17. The vector of claim 16 wherein the vector is a lentiviral vector.

18. The vector of claim 16 wherein the vector is an AAV vector.

19. A virion comprising a vector according to any one of claims 15-18.

20. A pharmaceutical composition comprising a synthetic NS-specific promoter according to claims 1-6, 10 or 13, an expression cassette according to claim 14, a vector according to any one of claims 15-18, or a virion according to claim 19.

21. A synthetic NS-specific promoter according to claims 1-6, 10 or 13, an expression cassette according to claim 14, a vector according to any one of claims 15-18, a virion according to claim 19, or a pharmaceutical composition according to claim 20 for use as a medicament.

22. A cell comprising a synthetic NS-specific promoter according to claims 1-6, 10 or 13, an expression cassette according to claim 14, a vector according to any one of claims 15-18, or a virion according to claim 19.

23. A synthetic NS-specific promoter according to claims 1-6, 10 or 13, an expression cassette according to claim 14, a vector according to any one of claims 15-18, a virion according to claim 19, or a pharmaceutical composition according to claim 20 for use in the manufacture of a pharmaceutical composition for the treatment of a medical condition or disease.

24. A method for producing an expression product, the method comprising providing a synthetic NS-specific expression cassette according to claim 14 in a NS cell and expressing the expression product present in the synthetic NS-specific expression cassette.

25. A method of expressing a therapeutic transgene in a NS cell, the method comprising introducing into the NS cell a synthetic NS-specific expression cassette according to claim 14, a vector according to any one of claims 15-18 or a virion according to claim 19.

26. The method of expressing a therapeutic transgene in a NS cell according to claim 25, wherein the expression cassette, the vector or the virion are introduced by intravenous or injection.

27. The method of claim 26, wherein the injection is in one of the cephalic, median or basilic veins.

28. A method of therapy of a subject in need thereof, preferably a human, the method comprising:
administering to the subject an expression cassette according to claim 14, a vector according to any one of claims 15-18, a virion according to claim 19, or a pharmaceutical composition according to claim 20, which comprises a sequence encoding a therapeutic product operably linked to a promoter according to any of claims 1-6, 10 or 13; and
expressing a therapeutic amount of the therapeutic product in the NS of said subject.


SEQUENCE LISTING (41 sequences)

SEQ ID NO: 1 - DNA, 795 bp, Artificial Sequence
  SP0013
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 2 - DNA, 810 bp, Artificial Sequence
  SP0014
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 3 - DNA, 916 bp, Artificial Sequence
  SP0026
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcactgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 4 - DNA, 931 bp, Artificial Sequence
  SP0027
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcacttgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 5 - DNA, 617 bp, Artificial Sequence
  SP0030
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 6 - DNA, 632 bp, Artificial Sequence
  SP0031
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcactgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 7 - DNA, 701 bp, Artificial Sequence
  SP0032
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 8 - DNA, 716 bp, Artificial Sequence
  SP0033
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 9 - DNA, 792 bp, Artificial Sequence
  SP0019
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 10 - DNA, 686 bp, Artificial Sequence
  SP0020
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggccgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 11 - DNA, 807 bp, Artificial Sequence
  SP0021
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 12 - DNA, 701 bp, Artificial Sequence
  SP0022
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggcctgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 13 - DNA, 913 bp, Artificial Sequence
  SP0028
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcactgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 14 - DNA, 928 bp, Artificial Sequence
  SP0029
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcacttgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 15 - DNA, 623 bp, Artificial Sequence
  CRM_SP0026
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcact

SEQ ID NO: 16 - DNA, 623 bp, Artificial Sequence; identical to SEQ ID NO: 15
  CRM_SP0027

SEQ ID NO: 17 - DNA, 620 bp, Artificial Sequence
  CRM_SP0028
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcact

SEQ ID NO: 18 - DNA, 620 bp, Artificial Sequence; identical to SEQ ID NO: 17
  CRM_SP0029

SEQ ID NO: 19 - DNA, 502 bp, Artificial Sequence
  CRE0005_GFAP (SYNP_AST_CRE0005_GFAP)
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 20 - DNA, 324 bp, Artificial Sequence
  CRE0007_GFAP (SYNP_AST_CRE0007_GFAP)
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcac

SEQ ID NO: 21 - DNA, 121 bp, Artificial Sequence
  CRE0012_Arc (SYNP_NEU_CRE0012_Arc)
agcgcgcagagtctgcatgcgtgaggaagctcctgggcgcgtcacagccgcgctattctcagcgtctctccttttatggctccggaagtgagctggggttgctggcagcctggctggcact

SEQ ID NO: 22 - DNA, 393 bp, Artificial Sequence
  CRE0001_S100B (SYNP_AST_CRE0001_S100B)
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggcc

SEQ ID NO: 23 - DNA, 408 bp, Artificial Sequence
  CRE0009_S100B (SYNP_AST_CRE0009_S100B)
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaa

SEQ ID NO: 24 - DNA, 499 bp, Artificial Sequence
  CRE0002_S100B (SYNP_AST_CRE0002_S100B)
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaa

SEQ ID NO: 25 - DNA, 293 bp, Artificial Sequence
  CRE0003_CEND1_mp (SYNP_NEU_CRE0003_CEND1_mp)
gggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 26 - DNA, 308 bp, Artificial Sequence
  CRE0009_SYN1_mp (SYNP_NEU_CRE0009_SYN1_mp)
tgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 27 - DNA, 359 bp, Artificial Sequence
  CRE0006_GFAP (SYNP_AST_CRE0006_GFAP)
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgcca

SEQ ID NO: 28 - DNA, 463 bp, Artificial Sequence
  CRE0008_GFAP (SYNP_AST_CRE0008_GFAP)
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 29 - DNA, 88 bp, Artificial Sequence
  CRE0001_CEND1_mp (SYNP_NEU_CRE0001_CEND1_mp)
gatcccacggttcccggcggggcggggcccggtcgcccctccccctccccgccctcctgcgccgggagcagtgcattgtgggaaactc

SEQ ID NO: 30 - DNA, 346 bp, Artificial Sequence
  CRE0002_CEND1_mp (SYNP_NEU_CRE0002_CEND1_mp)
gggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccgagctctcctccgcgttcgcagccgccgtcatcccgcggaggagcgcgcagcccc

SEQ ID NO: 31 - DNA, 468 bp, Artificial Sequence
  CRE0008_SYN1_mp (SYNP_NEU_CRE0008_SYN1_mp)
tgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 32 - DNA, 266 bp, Artificial Sequence
  CRE0013_SYN1_mp (SYNP_NEU_CRE0013_SYN1_mp)
tgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtct

SEQ ID NO: 33 - DNA, 529 bp, Artificial Sequence
  SP0011
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacatgagctctccccatagctgggctgcggcccaaccccaccccctcaggctatgccagggggtgttgccaggggcacccgggcatcgccagtctagcccactccttcataaagccctcgcatcccaggagcgagcagagccagagc

SEQ ID NO: 34 - DNA, 646 bp, Artificial Sequence
  SP0034
tccaaagaaaagccagataagtagctgattattgcatagagctgacagtatcacaggaagatcagtagtagcagctcaagtacaaaaaggttaattagcaatacttaataagaaaaactacctctggcaggtgaagagttaatccctggtcaattttaagctactctgctgagagtactaataagtgtaggggttggagccaatgagggtgaccccttccttgatgggaacagtcatcccttaggaactgccctggaaagcatcagccagccagaaaacagggaaagaggctgagaaaccgtggtaaccaagttttgctggcactttgtaaaatggtaactgcaactgccgaggctgtgcagagaatgctaataagcctaggacaacctgtaaagagtggacctagaaaatgtccacccgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 35 - DNA, 614 bp, Artificial Sequence
  SP0035
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacatcgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 36 - DNA, 695 bp, Artificial Sequence
  SP0036
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacattcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggccccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgag

SEQ ID NO: 37 - DNA, 386 bp, Artificial Sequence
  CRE0006_AQP4 (SYNP_AST_CRE0006_AQP4)
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacat

SEQ ID NO: 38 - DNA, 418 bp, Artificial Sequence
  CRE0008_AQP4 (SYNP_AST_TO_CRE0008v2_AQP4)
tccaaagaaaagccagataagtagctgattattgcatagagctgacagtatcacaggaagatcagtagtagcagctcaagtacaaaaaggttaattagcaatacttaataagaaaaactacctctggcaggtgaagagttaatccctggtcaattttaagctactctgctgagagtactaataagtgtaggggttggagccaatgagggtgaccccttccttgatgggaacagtcatcccttaggaactgccctggaaagcatcagccagccagaaaacagggaaagaggctgagaaaccgtggtaaccaagttttgctggcactttgtaaaatggtaactgcaactgccgaggctgtgcagagaatgctaataagcctaggacaacctgtaaagagtggacctagaaaatgtccacc

SEQ ID NO: 39 - DNA, 228 bp, Artificial Sequence
  CRE0015_GAP43_mp (SYNP_NEU_CRE0015_GAP43_mp)
cgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 40 - DNA, 309 bp, Artificial Sequence
  CRE0016_Eno2_mp (SYNP_NEU_CRE0016_Eno2_mp)
tcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggccccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgag

SEQ ID NO: 41 - DNA, 143 bp, Artificial Sequence
  CRE0004_GFAP_mp (SYNP_AST_CRE0004_GFAP_mp)
gagctctccccatagctgggctgcggcccaaccccaccccctcaggctatgccagggggtgttgccaggggcacccgggcatcgccagtctagcccactccttcataaagccctcgcatcccaggagcgagcagagccagagc